SEQUENCE LISTING (85 sequences)

SEQ ID NO: 1 - protein, 419 aa, synthetic construct
MASSLKAVLKHPDDFYPLLKLKMAAKKAEKQIPSQPHWAFSYSMLHKVSRSFALVIQQLNPQLRDAVCIFYLVLRALDTVEDDTSIAADIKVPILIAFHKHIYNRDWHFACGTKEYKVLMDQFHHVSTAFLELKRGYQEAIEDITMRMGAGMAKFICKEVETVDDYDEYCHYVAGLVGIGLSKLFHSSGTEILFSDSISNSMGLFLQKTNIIRDYLEDINEIPKSRMFWPREIWSKYVNKLEDLKYEENSEKAVQCLNDMVTNALIHIEDCLKYMSQLKDPAIFRFCAIPQIMAIGTLALCYNNIEVFRGVVKLRRGLTAKVIDRTKTMADVYQAFSDFSDMLKSKVDMHDPNAQTTITRLEAAQKICKDSGTLSNRKSYIVKRESSYSAALLALLFTILAILYAYLSANRPNKIKFTL

SEQ ID NO: 2 - protein, 438 aa, synthetic construct
MAKEEFDICIIGAGMAGATISAYLAPKGIKIALIDHCYKEKKRIVGELLQPGAVLSLEQMGLSHLLDGFEAQTVKGYALLQGNEKTTIPYPSQHEGIGLHNGRFLQQIRASALENSSVTQIHGKALQLLENERNEIIGVSYRESITSQIKSIYAPLTITSDGFFSNFRAHLSNNQKTVTSYFIGLILKDCEMPFPKHGHVFLSGPTPFICYPISDNEVRLLIDFPGEQLPRKNLLQEHLDTNVTPYIPECMRSSYAQAIQEGGFKVMPNHYMAAKPIVRKGAVMLGDALNMRHPLTGGGLTAVFSDIQILSAHLLAMPDFKNTDLIHEKIEAYYRDRKRANANLNILANALYAVMSNDLLKTAVFKYLQCGGANAQESIAVLAGLNRKHFSLIKQFCFLAVFGACNLLQQSISNIPKALKLLKDAFVIIKPLIKNELS

SEQ ID NO: 3 - protein, 770 aa, synthetic construct
MAWKLKVAQGNDPYLYSTNNFVGRQYWEFQPDAGTPEEREEVEKARKDYVNNKKLHGIHPCSDMLMRRQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 4 - protein, 770 aa, synthetic construct
MAWKLKVAQGNDPYLYSTNNFVGRQYWEFQPDAGTPEEREEVEKARKDYVNNKKLHGIHPCSDMLMRRQLIKESGIDLLSIPPLRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHICCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKVHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAMLGLMFGGQAERDPTPLHRAAKLLINAQMDNGDFPQQEITGVYCKNSMLHYAEYRNIFPLWALGEYRKRVWLPKHQQLKI

SEQ ID NO: 5 - protein, 770 aa, synthetic construct
MAWKLKVAQGNDPYLYSTNNFVGRQYWEFQPDAGTPEEREEVEKARKDYVNNKKLHGIHPCSDMLMRRQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERIQMPDGSWYGFWGICFLYGTFFALSGLASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 6 - protein, 770 aa, synthetic construct
MAWKLKVAQGNDPYLYSTNNFVGRQYWEFQPDAGTPEEREEVEKARKDYVNNKKLHGIHPCSDMLMRRQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGAEETRYITTGNGEKALQIMSWWAEDPNGDEFKHHLARIPDFLWIAEDGMTVQSFGSQLWDCILATQAIIATNMVEEYGDSLKKAHFFIKESQIKENPSGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 7 - protein, 770 aa, synthetic construct
MAWKLKVAQGNDPYLYSTNNFVGRQYWEFDPDAGTPEEREEVEKARKDYVNNKKLHGIHPCSDLLMRMQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 8 - protein, 770 aa, synthetic construct
MAWKLKVAQGNDPYLYSTNNFVGRQYWEFQPDAGTPEEREEVEKARKDYVNNKKLHGIHPCSDMLMRRQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCALATQAIIATNMVEEYGDSLKKAHFFIKESQIKENPRGDFKKMYRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 9 - protein, 770 aa, synthetic construct
MAWKLKVAQGNDPYLYSTNNFVGRQYWEFLPEAGTPEEREEVEKARKDYVNNKKLHGIHPCSDMLMRRQLIKESGIDLLSIPPVRLDENEQVNYDAVTTAVKKALRLNRAIQAHDGHWPAENSGSLLYTPPLIIALYISGTIDTTLTKQHKKELIRYVYNHQNEDGGWGSYIEGSSTMIGSVLSYVMLRLLGEGSAESDDGNGAVERGRKWILDHGGAAGIPSWGKTYLAVLGVYEWEGCNPLPPEFWLFPSFFPFHPAKMWIYCRCTYMPMSYLYGKRYHGPITDLVLSLRQEIYNIPYEQIKWNLQRHNCCKEDLYYPHTLVQDLVWDGLHYFSEPFLKRWPFNKLRKRGLKRVVELMRYGATETRFITTGCGEKALQIMSWWAEDPNGDEFKHHLARVPDFLWIAEDGMTVQSFGSQLWDCILATQAIIATNMVEEYGDSLKKAHFYIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIENSVVKAIRYLERNQMPDGSWYGFWGICFLYGTFFTLSGFASAGRTYDNSEAVRKGVKFFLSTQNEEGGWGESLESCPSEKFTPLKGNRTNLVQTSWAMLGLMFGGQAERDPTPLHRAAKLLINAQMDNGDFPQQEITGVYCKNSMLHYAEYRNIFPLWALGEYRKRVWLPKHQQLKI

SEQ ID NO: 10 - protein, 487 aa, synthetic construct
MAQDLRLILIIVGAIAIIALLVHGFWSYTKRIPQKENDSKAPLPPGQTGWPLIGETLNYLSCVKSGVSENFVKYRKEKYSPKVFRTSLLGEPMAILCGPEGNKFLYSTEKKLVQVWFPSSVEKMFPRSHGESNADNFSKVRGKMMFLLKVDGMKKYVGLMDRVMKQFLETDWNRQQQINVHNTVKKYTVTMSCRVFMSIDDEEQVTRLGSSIQNIEAGLLAVPINIPGTAMNRAIKTVKLLTREVEAVIKQRKVDLLENKQASQPQDLLSHLLLTANQDGQFLSESDIASHLIGLMQGGYTTLNGTITFVLNYLAEFPDVYNQVLKEQVEIANSKHPKELLNWEDLRKMKYSWNVAQEVLRIIPPGVGTFREAITDFTYAGYLIPKGWKMHLIPHDTHKNPTYFPSPEKFDPTRFEGNGPAPYTFTPFGGGPRMCPGIEYARLVILIFMHNVVTNFRWEKLIPNEKILTDPIPRFAHGLPIHLHPHN

SEQ ID NO: 11 - protein, 489 aa, synthetic construct
MAQDLRLILIIVGAIAIIALLVHGFAYFSYTKRIPQKENDSKAPLPPGQTGWPLIGETLNYLSCVKSGFSENFVKYRKEKYSPKVFRTSLLGEPMAILCGPEGNKFLYSTEKKLVQTWFPSSVEKMFPRSHGESNADNFSKVRGKMMFLLKVDGLKKYVGLMDRVMKQFLETDWNRQQQINVHNTVKKYTVTMSCRVFMSIDDEEQVRRLGSSIQNIEAGLLAVPINIPGTAMNRAIKTVKLLSREVEAVIKQRKVDLLENKQASQPQDLLSHLLLTANQDGQFLSESDIASHLIGLMQGGYTTLNGTITFVINYLAEFPDVYNQVLKEQVEIANSKHPKELLNWEDLRKMKYSWNVAQEVLRIIPPGVGTFREAITDFTYAGYLIPKGWKMHLIPHDTHKNPTYFPNPEKFDPTRFEGNGPAPYTFTPFGGGPRMCPGIEYARLVILIFIHNVVTNFRWEKLIPSEKILTDPIPRFAHGLPIHLHPHN

SEQ ID NO: 12 - protein, 512 aa, synthetic construct
MAQDLRLILIIVGAIAIIALLVHGFMAAAMVLFFSLSLLLLPLPLLLFAYFSYTKRIPQKENDSKAPLPPGQTGWPLIGETLNYLSCVKSGFSENFVKYRKEKYSPKVFRTSLLGEPMAILCGPEGNKFLYSTEKKLVQTWFPSSVEKMFPRSHGESNADNFSKVRGKMMFLLKVDGLKKYVGLMDRVMKQFLETDWNRQQQINVHNTVKKYTVTMSCRVFMSIDDEEQVRRLGSSIQNIEAGLLAVPINIPGTAMNRAIKTVKLLSREVEAVIKQRKVDLLENKQASQPQDLLSHLLLTANQDGQFLSESDIASHLIGLMQGGYTTLNGTITFVINYLAEFPDVYNQVLKEQVEIANSKQPKELLNWEDLRKMKYSWNVAQEVLRIIPPGVGTFREAITDFTYAGYLIPKGWKMHLIPHDTHKNPTYFPNPEKFDPTRFEGNGPAPYTFTPFGGGPRMCPGIEYARLVILIFIHNVVTNFRWEKLIPSEKILTDPIPRFAHGLPIHLHPHN

SEQ ID NO: 13 - protein, 491 aa, synthetic construct
MAQDLRLILIIVGAIAIIALLVHGFAYFSYQLFITKHQGNDSKTPRLPPGRTGWPLIGESLNYISTIKSGLLENFVTYRMGKYSPKVFRTSIFGETMAVLCGPEGNKLIFSNERKLVRVWFPSSVDKIFPRSHGETNAENFFKVRKMMFVLKVDALKKYVGLMDTAMKQFLRTDWNHRHQQINVYETVKKYTVMMACRVFMSIDDAEQLGKISNLIQHIEAGLFAVPINLPGTAMNRAIKTVELLSKDLEAVVKQRKVDLLNNKASPTQDLLSHLLLTANDDGRFLSESDIASHLLGLMQGGYSTLNVTITFIMNYLAELPDVYNQVLKEQVEIANSKSPKELLNWEDLRKMKYSWNVAQEVLRIRSPGVGTFREVIADFTYAGYLIPKGWKIPLIPQSTFKNPAYYPNPEKFDPTRFEGNGPAPYSYTPFGGGPRMCPGVEYARLAILIFMHNVVTNFKWEKLIPNEKIFTYPAPKFAHGLPIQLHPHNL

SEQ ID NO: 14 - protein, 492 aa, synthetic construct
MAQDLRLILIIVGAIAIIALLVHGFAYFSHQIFITKHRNTDSKIPLPPGPTGWPLIGESLNYLSTVKSGLLENFVTYRKEKYSTKVFRTSLFGESVAILCGAEGNKFLFSNERKLVRVWFPRSVEKIFAQSHAESNAESFYKIRKMMFILKADALKKYVGLMDTIMKQFLQTHWNHHLQTQINVHNTVMNYSLMLSCRVFMSIDDAEQVRKIGNSIHHIEAGLFAVPINLPGTAMNRAIKTVKLLSKEFEAVVKQRKADLLENKQAPPTQDLLSHLLLTPNEDGRFMSESDIARQLLGLVQGAYSTLNVVIAFIINYLAELPDVYDQVLKEQVEIAKSKNPKELLNWEDLSKMKYSWNVVQEVLRIRSPAIGVFREAINDFTYAGYLIPKGWKLHLIPVATHKNPTYFPNPEKFDPTRFEGSGPAPYTFTPFGGGPRMCPGVEYARLAILIFMHHAVTNFRWEKLIPNEQIFTFPVLSFANGLPIHLHPHNP

SEQ ID NO: 15 - protein, 491 aa, synthetic construct
MAQDLRLILIIVGAIAIIALLVHGFSYLISGHTPRRANENISSENFPLPPGRTGWPLIGESLDYFLKLRNCIPEKFVADRRDRYSTKVFKTSLLGEPMAIFCGVEGNKFLFSSETKLVQLWWPKAISKIFPKSSADYMKEDSTKVRKILQPFLKADALQKHVGVMDMLMKQHLDMDWNCREVKVSPAVTKYTFMLACRLFLSIEDLERVEELGKSFGYITAGIVSMAINVPGTAFNRAIKASKIMRRELEAMIQQRKIDLTENRSLAAQDLLSHMLLANDENDRFMTEFDIASHIVGLLHAATHTLNVALTFIVMYLAELPDVYNEVLREQMGIAESKEPEDLLNWKDIKKMKYSWNVANEVLRLRPPSFGTFREAITDFTYAGYMIPKGWKLHLIAQTTHKNPEYFPNPETFDPSRFEGNGPPPFTFVPFGGGPRMCPGNEYARLVMLVFMHNMVTKFRWKKVIPNEKVVIDPLPRPTQGLPVHLHPHKP

SEQ ID NO: 16 - protein, 489 aa, synthetic construct
MAQDLRLILIIVGAIAIIALLVHGFAYFSYTKRIPQKENDSKAPLPPGQTGWPLIGETLNYLSCVKSGVSENFVKYRKEKYSPKVFRTSLLGEPMAILCGPEGNKFLYSTEKKLVQVWFPSSVEKMFPRSHGESNADNFSKVRCKMMFLLKVDGMKKYVGLMDRVMKQFLESDWNRQQQINVHNTVKKYTVTMSCRVFMSIDDEEQVTRLGSSIQNIEAGLLAVPINIPGTAMNRAIKTVKLLTREVEAVIKQRKVDLLENKQASQPQDLLSHLLLTANQDGQFLSESDIASHLIGLMQGGYTTLNGTITFVLNYLAEFPDVYNQVLKEQVEIANSKHPKELLNWEDLRKMKYSWNVAQEVLRIIPPGVGTFREAITDFTYAGYLIPKGWKMHLIPHDTHKNPTYFPSPEKFDPTRFEGNGPAPYTFTPFGGGPRMCPGIEYARLVILIFMHNVVTNFRWEKLIPNEKILTDPIPRFAHGLPIHLHPHN

SEQ ID NO: 17 - protein, 474 aa, synthetic construct
MAQDLRLILIIVGAIAIIALLVHGFWNFKPSSQNKLPPGKTGWPIIGETLEFISCGQKGNPEKFVTQRMNKYSPDVFTTSLAGEKMVVFCGASGNKFIFSNENKLVVSWWPPAISKILTATIPSVEKSKALRSLIVEFLKPEALHKFISVMDRTTRQHFEDKWNGSTEVKAFAMSESLTFELACWLLFSINDPVQVQKLSHLFEKVKAGLLSLPLNFPGTAFNRGIKAANLIRKELSVVIKQRRSDKLQTRKDLLSHVMLSNGEGEKFFSEMDIADVVLNLLIASHDTTSSAMGSVVYFLADHPHIYAKVLTEQMEIAKSKGAEELLSWEDIKRMKYSRNVINEAMRLVPPSQGGFKVVTSKFSYANFIIPKGWKIFWSVYSTHKDPKYFKNPEEFDPSRFEGDGPMPFTFIPFGGGPRMCPGSEFARLEVLIFMHHLVTNFKWEKVFPNEKIIYTPFPFPENGLPIRLSPCTL

SEQ ID NO: 18 - protein, 474 aa, synthetic construct
MAQDLRLILIIVGAIAIIALLVHGFWNFKPSSQNKLPPGKTGWPIIGETLEFISCGQKGNPEKFVTQRMKKYSPDVFTTSLAGEKMVVFCGASGNKFIFSNENKLVVSWWPPAISKILTATIPSVEKSKALRSLIVEFLKPEALHKFISVMDRTTRQHFEDKWNGSTEVKAFAMSESLTFELACWLLFSINDPVQVQKLSHLFEKVKAGLLSLPLNFPGTAFNRGIKAANLIRKELSVMIKQRRSDKLQTRKDLLSHVMLSNGEGEKFFSEMDIADVVLNLLIASHDTTSSAMGSVVYFLADHPHIYAKVLTEQMEIAKSKGAGELLSWEDIKRMKYSRNVINEAMRLVPPSQGGFKVVTSKFSYANFIIPKGWKIFWSVYSTHKDPKYFKNPEEFDPSRFEGDGPMPFTFIPFGGGPRMCPGSEFARLEVLIFMHHLVTNFRWDKVFPNEKIIYTPFPSTENSRTIRLSPCTL

SEQ ID NO: 19 - protein, 475 aa, synthetic construct
MAQDLRLILIIVGAIAIIALLVHGFFWNFKPSSQNKLPPGKTGWPIIGETLEFISCGQKGNPEKFVTQRMKKYSPDVFTTSLAGEKMVVFCGASGNKFIFSNENKLVVSWWPPAISKILTATIPSVEKSKALRSLIVEFLKPEALHKFISVMDRTTRQHFEAKWNGSTEVKAFAMSETLTFELACWLLFSISDPVQVQKLSHLFEKVKAGLLSLPLNFPGTAFNRGIKAANLIRKELSVVIKQRRSDKSETRKDLLSHVMISNGEGEKFFSEMDIADVVLNILIASHDTTSSAMGSVVYFLADHPHIYAKVLAEQMEIAKSKGAGELLSWEDIKRMKYSRNVINEAMRLVPPSQGGFKVVTSKFSYANFIIPKGWKIFWSVYSTHKDPKYFKNPEEFDPSRFEGDGPMPFTFIPFGGGPRMCPGSEFARLEVLIFMHHLVTNFRWEKVFPNEKIIYTPFPFPENGLPIRLSPCTL

SEQ ID NO: 20 - protein, 472 aa, synthetic construct
MAQDLRLILIIVGAIAIIALLVHGFLRPSSQNKLPPGKTGWPIIGETLEYLSWGQKGCPEKFITQRMNKYSPHVFTTSLAGEKMAIFCGASGNKFMFSNENKLVVSWWPPAISKIVNSTKPSVEKSKAVRNLIVEFLKPEALHKFIPVMDRTTRLHFEAEWGGTTEVKAFALSEMLTFELACRLLCSIDDPVHVKTLSCLFAKVKAGLMSLPIDFPGTAFNSGIKAANLIRKDLSVLIEQRRSDKLQIRGDLLSHILISNGEDEKILSEMDIADVVLGLLIASHDTTSSVMASVVYFLTDHPGIYAKVLTEQMEIAKSKRAGDLLTWENIQRMKYSRNVINEVMRLVPPSQGGFKEVISEFSYADFIIPKGWKIFWSVHSTHKDPKYFKNPEEFDPPRFEGDGPMPFTFIPFGGGPRMCPGNEFARMEVLIFMHHLVMNFRWEKVFPNEKIIYTSFPFPEKGLPIRLSPCTL

SEQ ID NO: 21 - protein, 475 aa, synthetic construct
MAQDLRLILIIVGAIAIIALLVHGFFWNFKPSSQNKLPPGKTGWPIIGETLEFISWGQKGNPEKFVTQRMKKYSPDVFTTSLAGEKMVVFCGASGNKFIFSNENKLVVSWWPPAISKILTATIPSVEKSKALRSLIVEFLKPEALHKFISVMDRTTRQHFEAKWNGSTEVKAFAMSETLTFELACWLLFSINDPVQVQKLSHLFEKVKAGLLSLPLNFPGTAFNRGIKAANLIRKELSVVIKQRRSDKSETRKDLLSHVMISNGEGEKFFSEMDIADVVLNILIASHDTTSSAMGSVVYFLADHPHIYAKVLTEQMEIAKSKGAGELLSWEDIKRMKYSRNVINEAMRLVPPSQGGFKVVTSKFSYANFIIPKGWKIFWSVYSTHKDPKYFKNPEEFDPSRFEGDGPMPFTFIPFGGGPRMCPGSEFARLEVLIFMHHLVTNFRWEKVFPNEKIIYTPFPFPENGLPIRLSPCTL

SEQ ID NO: 22 - protein, 709 aa, synthetic construct
MAQSSSVKVSTFDLMSAILRGRSMDQTNVSFESGESPALAMLIENRELVMILTTSVAVLIGCFVVLLWRRSSGKSGKVTEPPKPLMVKTEPEPEVDDGKKKVSIFYGTQTGTAEGFAKALAEEAKVRYEKASFKVIDLDDYAADDEEYEEKLKKETLTFFFLATYGDGEPTDNAARFYKWFMEGKERGDWLKNLHYGVFGLGNRQYEHFNRIAKVVDDTIAEQGGKRLIPVGLGDDDQCIEDDFAAWRELLWPELDQLLQDEDGTTVATPYTAAVLEYRVVFHDSPDASLLDKSFSKSNGHAVHDAQHPCRANVAVRRELHTPASDRSCTHLEFDISGTGLVYETGDHVGVYCENLIEVVEEAEMLLGLSPDTFFSIHTDKEDGTPLSGSSLPPPFPPCTLRRALTQYADLLSSPKKSSLLALAAHCSDPSEADRLRHLASPSGKDEYAQWVVASQRSLLEVMAEFPSAKPPIGAFFAGVAPRLQPRYYSISSSPRMAPSRIHVTCALVFEKTPVGRIHKGVCSTWMKNAVPLDESRDCSWAPIFVRQSNFKLPADTKVPVLMIGPGTGLAPFRGFLQERLALKEAGAELGPAILFFGCRNRQMDYIYEDELNNFVETGALSELIVAFSREGPKKEYVQHKMMEKASDIWNMISQEGYIYVCGDAKGMARDVHRTLHTIVQEQGSLDSSKTESMVKNLQMNGRYLRDVW

SEQ ID NO: 23 - protein, 417 aa, synthetic construct
MGSLGAILRHPDDFYPLLKLKMAARHAEKQIPPEPHWGFCYTMLHKVSRSFALVIQQLAPELRNAICIFYLVLRALDTVEDDTSIQTDIKVPILKAFHCHIYNRDWHFSCGTKDYKVLMDQFHHVSTAFLELGKGYQEAIEDITKRMGAGMAKFICKEVETVDDYDEYCHYVAGLVGLGLSKLFHASDLEDLAPDSLSNSMGLLLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTQTMADVYGAFFDFSVMLKAKVNSSDPNATKTLSRIEAIQKTCEQSGLLNKRKLYAVKSEPMFNPTLIVILFSLLCIILAYLSAKRLPANQPV

SEQ ID NO: 24 - protein, 417 aa, synthetic construct
MGSLGAILRHPDDFYPLLKLKMAARHAEKQIPPEPHWGFCYTMLHKVSRSFALVIQQLAPELRNAICIFYLVLRALDTVEDDTSIQTDIKVPILKAFHCHIYNRDWHFSCGTKDYKVLMDQFHHVSTAFLELGKGYQEAIEDITKRMGAGMAKFICKEVETVDDYDEYCHYVAGLVGLGLSKLFHASDLEDLAPDSLSNSMGLLLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTQTMADVYGAFFDFSVMLKAKVNNSDPNATKTLSRIEAIQKTCEQSGLLNKRKLYAVKSEPMFNPTLIVILFSLLCIILAYLSAKRLPANQPV

SEQ ID NO: 25 - protein, 417 aa, synthetic construct
MGSLGAILKHPDDFYPLLKLKIAARHAEKQIPPEPHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGKGYQEAIEDITKRMGAGMAKFICKEVETVDDYDEYCHYVAGLVGLGLSKLFHAAELEDLAPDSLSNSMGLFLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTKTMADVYGAFFDFSVMLKAKVNSNDPNASKTLSRIEAIQKTCKQSGILNRRKLYVVRSEPMFNPAVIVILFSLLCIILAYLSAKRLPANQSV

SEQ ID NO: 26 - protein, 417 aa, synthetic construct
MGSLGAILKHPDDFYPLLKLKMAARHAEKQIPPESHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGKGYQEAIEDITKRMGAGMAKFICKEVETVDDYDEYCHYVAGLVGLGLSKLFHAAELEDLAPDSLSNSMGLFLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTKTMADVYGAFFDFSVMLKAKVNSNDPNASKTLSRIEAIQQTCQQSGLMNKRKLYVVRSEPMYNPAVIVILFSLLCIILAYLSAKRLPANQSV

SEQ ID NO: 27 - protein, 417 aa, synthetic construct
MGSLGAILKHPDDFYPLLKLKMAARHAEKQIPPESHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGKGYQEAIEDITKRMGAGMAKFICKEVETVDDYDEYCHYVAGLVGLGLSKLFHAAELEDLAPDSLSNSMGLFLQKTNIIRDYLEDINEIPKSRMFWPREIWGKYADKLEDFKYEENSVKAVQCLNDLVTNALNHVEDCPKYMSNLRDLSIFRFCAIPQIMAIGTLALCYNNVEVFRGVVKMRRGLTAKVIDRTKTMADVYGAFFDFSVMLKAKVNSNDPNASKTLSRIEAIQQTCQQSGLMNKRKLYVVRSEPMYNPAVIVILFSLLCIILAYLSAKRLPANQSV

SEQ ID NO: 28 - protein, 417 aa, synthetic construct
MGSLGAILRHPDDIYPLLKLKMAARHAEKQIPPESHWGFCYTMLHKVSRSFALVIQQLKPELRNAVCIFYLVLRALDTVEDDTSIQTDIKVPILKAFHCHIYNRDWHFSCGTKDYKVLMDEFHHVSTAFLELGRGYQEAIEDITKRMGAGMAKFICKEVETVEDYDEYCHYVAGLVGLGLSKLFHASKSENLAPDSLSNSMGLFLQKTNIIRDYLEDINEIPKSRMFWPREIWSKYADKLEDFKYEKNSVKAVQCLNDLVTNALTHVEDCLEYMSNLKDLSIFRFCAIPQIMAIGTLALCYNNVDVFRGVVKMRRGLTAKVIYRTKTMADVYGAFFDFSVMLKAKVNSSDPNASKTLTRIEAIQKTCKQSGLLNKRELYAVRSEPMCNPAAIVVLFSLLCIILAYLSAKLLPANQPV

SEQ ID NO: 29 - protein, 417 aa, synthetic construct
MGSLGAILSHPDDLYPLLKLKMAAKHAEKQIPPDPHWGFCFSMLHKVSRSFALVIQQLKPELRNAVCIFYLVLRALDTVEDDTGIHPDIKVPILQAFHCHIYNRDWHFSCGTKHYKVLMDEFHHVSTAFLELGKGYQEAIEDVTERMGAGMAKFICKEVETVDDYDEYCHYVAGLVGLGLSKLFHAAELEDLAPDSLSNSMGLFLQKTNIIRDYLEDINEIPKSRMFWPREIWNKYADKLEDFKYEENSVKAVQCLNDLVTNALNHVEDCLKYMSNLKDLSTFRFCAIPQIMAIGTLALCYDNVEVFRGVVKMRRGLTAKIIDRTKKIADVYGAFFDFSVMLKAKVNSSDPNAAKTLSRIEAIEKTCKESGLLNKRKLYVIRSEPLFNPAVLVILFSLICILLAYLSAKRLPANQPV

SEQ ID NO: 30 - protein, 415 aa, synthetic construct
MGSLGAILKHPDDFYPLLKLKFAARHAEKQIPPEPHWAFCYSMLHKVSRSFGLVIQQLGPQLRDAVCIFYLVLRALDTVEDDTSIPTEVKVPILMAFHRHIYDKDWHFSCGTKEYKVLMDEFHHVSNAFLELGSGYQEAIEDITMRMGAGMAKFICKEVETIDDYDEYCHYVAGLVGLGLSKLFHASGAEDLATDSLSNSMGLFLQKTNIIRDYLEDINEIPKSRMFWPRQIWSKYVDKLEDLKYEENSAKAVQCLNDMVTDALVHAEDCLKYMSDLRDPAIFRFCAIPQIMAIGTLALCFNNTQVFRGVVKMRRGLTAKVIDRTKTMSDVYGAFFDFSCLLKSKVDNNDPNATKTLSRLEAIQKTCKESGTLSKRKSYIIESESGHNSALIAIIFIILAILYAYLSSNLLLNKQ

SEQ ID NO: 31 - protein, 412 aa, synthetic construct
MGALSTMLKHPDDIYPLLKLKIASRQIEKQIPAEPHWAFCYTMLQKVSRSFALVIQQLGTELRNAVCLFYLVLRALDTVEDDTSVATDVKVPILLAFHRHIYDPDWHFACGTNNYKVLMDEFHHVSTAFLELGTGYQEAIEDITKRMGAGMAKFILKEVETIDDYDEYCHYVAGLVGLGLSKLFHAAGKEDLASDSLSNSMGLFLQKTNIIRDYLEDINEIPKSRMFWPRQIWSKYVNKLEDLKYEENSEKAVQCLNDMVTNALIHMEDCLKYMAALRDPAIFKFCAIPQIMAIGTLALCYNNIEVFRGVVKMRRGLTAKVIDRTKSMDDVYGAFFDFSSILKSKVDKNDPNATKTLSRVEAVQKLCRDSGALSKRKSYIANREQSYNSTLIVALFIILAIIYAYLSASPRI

SEQ ID NO: 32 - protein, 408 aa, synthetic construct
MDQRSEDEFYPLLKLKIVARNAEKQIPPEPHWAFCYTMLHKVSRSFALVIQQLGIELRNAVCIFYLVLRALDTVEDDTSIETDVKVPILIAFHRHIYDRDWHFSCGTKEYKVLMGQFHHVSTAFLELGKNYQEAIEDITKRMGAGMAKFICKEVETIDDYDEYCHYVAGLVGLGLSKLFHASGSEDLAPDDLSNSMGLFLQKTNIIRDYLEDINEIPKSRMFWPRQIWSEYVNKLEDLKYEENSVKAVQCLNDMVTNALMHAEDCLTYMAALRDPPIFRFCAIPQIMAIGTLALCYNNIEVFRGVVKMRRGLTAKVIDRTKTMADVYGAFFDFASMLEPKVDKNDPNATKTLSRLEAIQKTCRESGLLSKRKSYIVNDESGYGSTMIVILVIMVSIIFAYLSANHHNS

SEQ ID NO: 33 - protein, 415 aa, synthetic construct
MGSLAAMLRHPDDVYPLVKLKMAARHAEKQIPPEPHWAFCYTMLHKVSRSFGLVIQQLGTELRNAVCIFYLVLRALDTVEDDTSIATEVKVPILLAFHHHIYDRDWHFSCGTREYKVLMDEFHHVSTAFLELGKGYQEAIEDITMRMGAGMAKFICKEVETIDDYDEYCHYVAGLVGLGLSKLFHASGLEDLAPDSLSNSMGLFLQKTNIIRDYLEDINEIPKSRMFWPRQIWSKYVNKLEDLKYEKNSVKSVQCLNDMVTNALIHVDDCLKYMSALRDPAIFRFCAIPQIMAIGTLALCYNNIEVFRGVVKMRRGLTAKVIDQTKTISDVYGAFFDFSCMLKSKVEKNDPNSTKTLSRIEAIQKTCRESGTLSKRKSYILRSKRTHNSTLIFVLFIILAILFAYLSANRPPINM

SEQ ID NO: 34 - protein, 410 aa, synthetic construct
MGSLGAILKHPDDFYPLLKLKMAAKHAEKQIPAQPHWGFCYSMLHKVSRSFSLVIQQLGTELRDAVCIFYLVLRALDTVEDDTSIPTDVKVPILIAFHKHIYDPEWHFSCGTKEYKVLMDQIHHLSTAFLELGKSYQEAIEDITKKMGAGMAKFICKEVETVDDYDEYCHYVAGLVGLGLSKLFDASGFEDLAPDDLSNSMGLFLQKTNIIRDYLEDINEIPKSRMFWPRQIWSKYVNKLEDLKYEENSVKAVQCLNDMVTNALIHMDDCLKYMSALRDPAIFRFCAIPQIMAIGTLALCYNNVEVFRGVVKMRRGLTAKVIDRTRTMADVYRAFFDFSCMMKSKVDRNDPNAEKTLNRLEAVQKTCKESGLLNKRRSYINESKPYNSTMVILLMIVLAIILAYLSKRAN

SEQ ID NO: 35 - protein, 413 aa, synthetic construct
MGSLGAILKHPDDFYPLMKLKMAARRAEKNIPPEPHWGFCYSMLHKVSRSFALVIQQLDTELRNAVCIFYLVLRALDTVEDDTSIATEVKVPILMAFHRHIYDRDWHFSCGTKEYKVLMDEFHHVSTAFSELGRGYQEAIEDITMRMGAGMAKFICKEVETIDDYDEYCHYVAGLVGLGLSKLFHASGSEDLASDSLSNSMGLFLQVFLLTCIKTNIIRDYLEDINEIPKSRMFWPRQIWSKYVNKLEDLKDKENSVKAVECLNDMVTNALIHVEDCLTYMSALRDPSIFRFCAIPQIMAIGTLALCYNNIEVFRGVVKMRRGLTAKVIDRTKTMSDVYGGFFDFSCMLKSKVNKSDPNAMKALSRLEAIQKICRESGTLNKRKSYIIKSEPRYNSTLVFVLFIILAILFAYL

SEQ ID NO: 36 - protein, 414 aa, synthetic construct
MGSLGAILKHPDDFYPLLKLKFAARHAEKQIPPEPHWAFCYSMLHKVSRSFGLVIQQLDAQLRDAVCIFYLVLRALDTVEDDTSIPTEVKVPILMAFHRHIYDKDWHFSCGTKEYKVLMDEFHHVSNAFLELGSGFQEAIEDITMRMGAGMAKFICKEVETIDDYDEYCHYVAGLVGLGLSKLFHASGAEDLATDSLSNSMGLFLQKTNIIRDYLEDINEIPKSRMFWPRQIWSKYVDKLENLKYEENSAKAVQCLNDMVTNALLHAEDCLKYMSNLRDPAIFRFCAIPQIMAIGTLALCFNNIQVFRGVVKMRRGLTAKVIDRTKTMSDVYGAFFDFSCLLKSKVDNNDPNATKTLSRLEAIQKTCKESGTLSKRKSYIIESKSAHNSALIAIIFIILAILYAYLSSNLPNNQ

SEQ ID NO: 37 - protein, 384 aa, synthetic construct
MLNNSLFSRLEEIPALLKLKLGSKDYYKNNNSETLTCDNLRYCFDTLNKVSRSFATVIKQLPNELGNNVCVFYLILRALDSIEDDMNLPKELKIKLLREFHKKNYESGWNISGVGDKKEHVELLENYDKVIQSFLAIDQKNQLIITDICRKVGAGMANFVKAEIESVEDYNLYCHHVAGLVGIGLSRMFISSGLENDDFLNQDEISNSMGLFLQKTNIVRDYREDLDEGRMFWPKDIWHVYGSKINDFAINPTHDQSVLCLNHMLNNALTHATDCLAYLKHLRNENIFKFCAIPQVMAMATLCKIYSNPDVFIKNVKIRKGLAAKLILNTTSMDEVIKVYKDMLLVIESKISSDNNPVSAETIQLLKQIREYFNDETLIVRKIA

SEQ ID NO: 38 - protein, 383 aa, synthetic construct
MLNSSLFSRLEEIPALLKLKLGSINNYKNNNSENLTSKNLRYCFDTLNKVSRSFASVIKQLPNELMVNVCLFYLILRALDSIEDDMNLPKDFKINLLREFLDKNYEPGWKISGVGDKKEYVELLENYDKVIQVFLDIDPKNQLIITDICRKMGAGMAHFVEAEINSVKDYNLYCYHVAGLVGIGLSKMFLASGLENCDYLNQEEISSSMGLFLQKTNIVRDYKEDMEENRIFWPKEIWRTYASKFSDFSINPQHETSISCLNHMVNDALGHVIDCLEYLRHLRNENIFKFCAIPQVMAMATLCKVYNNPDVFIKTVKIRKGLAAKLILNTTSMDEVIKVYKGLLLDIENKIPLHNPTSDETLRLIKNIRSYCNNETMVVSKTA

SEQ ID NO: 39 - protein, 528 aa, synthetic construct
MVDQCALGWILASALGLVIALCFFVAPRRNHRGVDSKERDECVQSAATTKGECRFNDRDVDVIVVGAGVAGSALAHTLGKDGRRVHVIERDLTEPDRIVGELLQPGGYLKLIELGLQDCVEEIDAQRVYGYALFKDGKNTRLSYPLENFHSDVSGRSFHNGRFIQRMREKAASLPNVRLEQGTVTSLLEEKGTIKGVQYKSKNGEEKTAYAPLTIVCDGCFSNLRRSLCNPMVDVPSYFVGLVLENCELPFANHGHVILGDPSPILFYQISRTEIRCLVDVPGQKVPSIANGEMEKYLKTVVAPQVPPQIYDSFIAAIDKGNIRTMPNRSMPAAPHPTPGALLMGDAFNMRHPLTGGGMTVALSDIVVLRNLLKPLKDLSDASTLCKYLESFYTLRKPVASTINTLAGALYKVFCASPDQARKEMRQACFDYLSLGGIFSNGPVSLLSGLNPRPLSLVLHFFAVAIYGVGRLLLPFPSVKGIWIGARLIYSASGIIFPIIRAEGVRQMFFPATVPAYYRSPPVFKPIV

SEQ ID NO: 40 - protein, 524 aa, synthetic construct
MVDQCALGWILASVLGAAALYFLFGRKNGGVSNERRHESIKNIATTNGEYKSSNSDGDIIIVGAGVAGSALAYTLGKDGRRVHVIERDLTEPDRIVGELLQPGGYLKLTELGLEDCVDDIDAQRVYGYALFKDGKDTRLSYPLEKFHSDVAGRSFHNGRFIQRMREKAASLPKVSLEQGTVTSLLEENGIIKGVQYKTKTGQEMTAYAPLTIVCDGCFSNLRRSLCNPKVDVPSCFVGLVLENCDLPYANHGHVILADPSPILFYRISSTEIRCLVDVPGQKVPSISNGEMANYLKNVVAPQIPSQLYDSFVAAIDKGNIRTMPNRSMPADPYPTPGALLMGDAFNMRHPLTGGGMTVALSDVVVLRDLLKPLRDLNDAPTLSKYLEAFYTLRKPVASTINTLAGALYKVFCASPDQARKEMRQACFDYLSLGGIFSNGPVSLLSGLNPRPISLVLHFFAVAIYGVGRLLIPFPSPKRVWIGARIISGASAIIFPIIKAEGVRQMFFPATVAAYYRAPRVVKGR

SEQ ID NO: 41 - protein, 526 aa, synthetic construct
MVDECALGWILAAALGAVIALCLFVAPKTNNQDGGVDSKATPECVQTTNGECRSDGDSDVIIVGAGVAGSALAHTLGKDGRRVHVIERDLTEPDRIVGELLQPGGYLKLIELGLADCVEEIDAQRVYGYALFKDGKNTRLSYPLEKFHSDVSGRSFHNGRFIQRMREKADSLPNVRLEQGTVTSLLEEKGTIKGVQYKSKDGKEKTAYAPLTIVCDGCFSNLRRSLCNPMVDVPSCFVGLVLENCQLPFANHGHVVLGDPSPILFYPISSTEIRCLVDVPGQKVPSISNGEMEKYLKTVVAPQVPPQIYDAFIAAIDKGNIRTMPNRSMPAAPHPTPGALLMGDAFNMRHPLTGGGMTVALSDIVVLRNLLKPLKDLHDAPTLCKYLESFYTLRKPVASTINTLAGALYKVFCASPDQARKEMRQACFDYLSLGGMFSNGPVSLLSGLNPRPLSLVLHFFAVAIYGVGRLLFPFPSPKGIWIGARLIYSASGIIFPIIKAEGVRQMFFPATVPAYYRSPPALKPVA

SEQ ID NO: 42 - protein, 529 aa, synthetic construct
MVDYCAFGWILAAVLGLAIALSFFVSPRRNRRGGADSTPRSEGVRSSSTTNGECRSVDGDADVIIVGAGVAGSALAHTLGKDGRLVHVIERDLTEPDRIVGELLQPGGYLKLIELGLQDCVEEIDAQKVYGYALFKDGKNTQLSYPLEKFQSDVSGRSFHNGRFIQRMREKAASLPNVRLEQGTVTSLLEEKGTIKGVQYKSKNGEEKTAYAPLTIVCDGCFSNLRRSLCKPMVDVPSCFVGLVLENCQLPFANHGHVVLGDPSPILFYPISSTEIRCLVDVPGQKIPSISNGEMEKYLKTIVAPQVPPQIHDAFIAAIDKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASPDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRSPPVHKSIA

SEQ ID NO: 43 - protein, 529 aa, synthetic construct
MVDYCAFGWILAAVLGLAIALSFFVSPRRNRRGGADSTPRSEGVRSSSTTNGECRSVDCDADVIIVGAGVAGSALAHTLGKDGRLVHVIERDLTEPDRIVGELLQPGGYLKLIELGLQDCVEEIDAQKVYGYALFKDGKNTQLSYPLEKFQSDVSGRSFHNGRFIQRMREKAASLPNVRLEQGTVTSLLEEKGTIKGVQYKSKNGEEKTAHAPLTIVCDGCFSNLRRSLCKPMVDVPSCFVGLVLENCQLPFANHGHVVLGDPSPILFYPISSTEIRCLVDVPGQKVPSISNGEMEKYLKTIVAPQVPPQIHDAFIAAIDKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASPDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRSPPVLKTIA

SEQ ID NO: 44 - protein, 531 aa, synthetic construct
MMVDHCAFAWILDVVLGLVVAVTFFVAAPRRNRRGGTDSTASKDCVISTAIANGECKPDDADAEVIIVGAGVAGSALAYTLGKDGRRVHVIERDLTEPDRIVGEFLQPGGYLKLIELGLGDCVEEIDAQKLYGYALFKDGKNTRVSYPLGNFHSDVSGRSFHNGRFIQRMREKAASLPNVRLEQGTVTSLLETKGTIKGVQYKSKNGEEKTAYAPLTIVCDGCFSNLRRSLCKPMVDVPSCFVGLVLENCQLPFANHGHVVLGDPSPILFYPISSTEIRCLVDVPGQKVPSISNGDMEKYLKTVVAPQVPPQIHDAFIAAIEKGNVRTMPNRSMPAAPHPTPGALLMGDAFNMRHPLTGGGMTVALSDIVVLRNLLKPLKDLNDASTLCKYLESFYTLRKPVASTINTLAGALYKVFCASPDQARKEMRQACFDYLSLGGVFSNGPISLLSGLNPRPSSLVLHFFAVAIYGVGRLLLPFPSLKGIWIGARLIYSASGIILPIIKAEGVRQMFFPATVPAYYRSPPVHKPIT

SEQ ID NO: 45 - protein, 528 aa, synthetic construct
MVDHCTFGWIFSAFLAFVIAFSFFLSPRKNRRGRGTNSTPRRDCLSSSATTNGECRSVDGDADVIIVGAGVAGSALAHTLGKDGRRVHVIERDLTEPDRIVGELLQPGGYLKLIELGLQDCVEEIDAQKVYGYALFKDGKSTRLSYPLENFQSDVSGRSFHNGRFIQRMREKAAFLPNVRLEQGTVTSLLEEKGTITGVQYKSKNGEQKTAYAPLTIVCDGCFSNLRRSLCNPMVDVPSCFVGLVLENCQLPYANLGHVVLGDPSPILFYPISSTEIRCLVDVPGQKVPSISNGEMEKYLKTVVAPQVPPQIHDAFIAAIEKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASSDQARKEMRQACFDYLSLGGIFSNGPVSLLSGLNPRPLSLVLHFFAVAIYGVGRLLLPFPSPKGIWIGARLVYSASGIIFPIIKAEGVRQMFFPATVPAYYRTPPVFNS

SEQ ID NO: 46 - protein, 528 aa, synthetic construct
MVDHCAFGWIFSALLAFPIALSLFLSPWRNRRVRGTDSTPRSASVSSSATTNGECRSVDGDADVVIVGAGVAGSALAHTLGKDGRRVHVIERDLTEPDRIVGELLQPGGYLKLIELGLQDCVEEIDAQKVYGYALFKDGKNTRLSYPLENFHSDVSGRSFHNGRFIQRMREKAASLPNVRLEQGTVTSLLEEKGTITGVQYKSKNGEQKTAYAPLTIVCDGCFSNLRRSLCTPMVDVPSYFVGLVLENCQLPYANLGHVVLGDPSPILFYPISSTEIRCLVDVPGQKVPSISNGEMEKYLKTVVAPQVPPQIHDAFIAAIEKGNIRTMPNRSMPAAPQPTPGALLMGDAFNMRHPLTGGGMTVALSDIVVLRNLLKPLKDLNDAPTLCKYLESFYTLRKPVASTINTLAGALYKVFCASPDQARKEMRQACFDYLSLGGIFSNGPVSLLSGLNPRPLSLVLHFFAVAIYGVGRLLLPFPSLKGIWIGARLVYSASGIIFPIIKAEGVRQMFFPATVPAYYRTPPVLNS

SEQ ID NO: 47 - protein, 533 aa, synthetic construct
MMVEHCAYGWILAAVLGLVVAVTFFVAVPRRNRRGGTDSTASKDCVISPAIANGECEPEDADADADVIIVGAGVAGSALAHTLGKDGRRVHVIERDLTEPDRIVGEFLQPGGHLKLIELGLGDCVEEIDAQKLYGYALFKDGKNTRVSYPLGNFHSDVSGRSFHNGRFIQRMREKAASLPNVRLEQGTVTSLLEKKGTIKGVQYKSKNGEEKTAYAPLTIVCDGCFSNLRRSLCKPMVDVPSCFVGLVLENCRLPFANHGHVVLGDPSPILFYPISSTEIRCLVDVPGQKVPSIPNGDMEKYLKTVVAPQVPPQIHDAFIAAIEKGNIRTMPNRSMPAAPHPTPGALLMGDAFNMRHPLTGGGMTVALSDIVVLRNLLKPLKDLNDAPTLCKYLESYYTLRKPVASTINTLAGALYKVFCASPDQARKEMRQACFDYLSLGGVFSNGPISLLSGLNPRPSCLVLHFFAVAIYGVGRLLLPFPSLKGIWIGARLIYSASGIILPIIKAEGVRQMFFPATVPAYYRSPPVHKPIT

SEQ ID NO: 48 - protein, 522 aa, synthetic construct
MLDQCPLGWILASVLGLFVLCNLIVKNRNSKASLEKRSECVKSIATTNGECRSKSDDVDVIIVGAGVAGSALAHTLGKDGRRLHVIERDLTEPDRIVGELLQPGGYLKLIELGLQDCVEEIDAQRVFGYALFKDGKDTRLSYPLEKFHSDVSGRSFHNGRFIQRMREKSASLPNVRLEQGTVTSLLEEKGTIKGVQYKTKTGQELTAFAPLTIVCDGCFSNLRRSLCNPKVDVPSCFVGLVLENCELPYANHGHVILADPSPILFYPISSTEVRCLVDVPGQKVPSISNGEMAKYLKSVVAPQIPPQIYDAFIAAVDKGNIRTMPNRSMPASPFPTPGALLMGDAFNMRHPLTGGGMTVALSDIVVLRDLLKPLGDLNDAATLCKYLESFYTLRKPVASTINTLAGALYKVFCASPDQARKEMRQACFDYLSLGGIFSTGPVSLLSGLNPRPLSLVLHFFAVAIYGVGRLLLPFPSPKRIWIGARLISGASGIIFPIIKAEGVRQMFFPATVPAYYRAAPVE

SEQ ID NO: 49 - protein, 524 aa, synthetic construct
MADPYTMGWILASLLGLFALYYLFVNNKNHREASLQESGSECVKSVAPVKGECRSKNGDADVIIVGAGVAGSALAHTLGKDGRRVHVIERDLAEPDRIVGELLQPGGYLKLIELGLQDCVEEIDSQRVYGYALFKDGKDTRLSYPLEKFHSDVSGRSFHNGRFIQRMREKAASLPNVQLEQGTVTSLLEENGTIKGVQYKTKTGQELTAYAPLTIVCDGCFSNLRRSLCIPKVDVPSCFVGLVLENCNLPYANHGHVVLADPSPILFYPISSTEVRCLVDVPGQKVPSISNGEMAKYLKTVVASQIPPQIYDSFVAAVDKGNIRTMPNRSMPAAPHPTPGALLMGDAFNMRHPLTGGGMTVALSDIVVLRDLLKPLRDLNDSVTLCKYLESFYTLRKPVASTINTLAGALYKVFCASPDQARKEMREACFDYLSLGGVFSEGPVSLLSGLNPRPLSLVCHFFAVAIYGVGRLLLPFPSPKRLWIGARLISGASGIIFPIIRAEGVRQMFFPATIPAYYRAPRPN

SEQ ID NO: 50 - protein, 527 aa, synthetic construct
MVDPYALGWSFASVLMGLVALYILVDKKNRSRVSSEARSEGVESVTTTTSGECRLTDGDADVIIVGAGVAGSALAHTLGKDGRRVHVIERDLTEPDRIVGELLQPGGYLKLIELGLEDCVEDIDAQRVFGYALFKDGKNTRLSYPLEKFHSDVSGRSFHNGRFIQRMREKAASLLNVRLEQGTVTSLLEENGTVKGVQYKTKDGNELTAHAPLTIVCDGCFSNLRRSLCNPQVDVPSSFVGLVLENCELPYANHGHVILADPSPILFYPISSTEVRCLVDVPGKKVPSIANGEMEKYLKNMVAPQLPPEIYDSFVAAVDRGNIRTMPNRSMPAAPHPTPGALLMGDAFNMRHPLTGGGMTVALSDIVVLRDLLKPLRDLNDAPTLCKYLESFYTLRKPVASTINTLAGALYKVFCASPDRARKEMRQACFDYLSLGGVFSMGPVSLLSGLNPRPLSLVLHFFAVAVYGVGRLLVPFPSPSRIWIGARLISGASAIIFPIIKAEGVRQMFFPATVPAYYRAPPVKRDH

SEQ ID NO: 51 - protein, 524 aa, synthetic construct
MVDQCALGWILASVLGASALYLLFGKKNCGVLNERRRESLKNIATTNGECKSSNSDGDIIIVGAGVAGSALAYTLAKDGRQVHVIERDLSEPDRIVGELLQPGGYLKLTELGLEDCVDDIDAQRVYGYALFKDGKDTRLSYPLEKFHSDVSGRSFHNGRFIQRMREKAASLPNVRLEQGTVTSLLEENGTIKGVQYKNKSGQEMTAYAPLTIVCDGCFSNLRRSLCNPKVDVPSCFVGLILENCDLPYANHGHVILADPSPILFYPISSTEIRCLVDVPGQKVPSISNGEMANYLKNVVAPQIPPQLYNSFIAAIDKGNIRTMPNRSMPADPYPTPGALLMGDAFNMRHPLTGGGMTVALSDIVVLRDLLKPLRDLNDAPTLCKYLEAFYTLRKPVASTINTLAGALYKVFCASPDQARKEMRQACFDYLSLGGIFSNGPVSLLSGLNPRPLSLVLHFFAVAIYGVGRLLIPFPSPKRVWIGARLISGASAIIFPIIKAEGVRQMFFPKTVAAYYRAPPVVRER

SEQ ID NO: 52 - protein, 524 aa, synthetic construct
MVDQCALGWILASVLGASALYLLFGKKNCGVSNERRRESLKNIATTNGECKSSNSDGDIIIVGAGVAGSALAYTLAKDGRQVHVIERDLSEPDRIVGELLQPGGYLKLTELGLEDCVDEIDAQRVYGYALFKDGKDTRLSYPLEKFHSDVSGRSFHNGRFIQRMREKAASLPNVRLEQGTVTSLLEENGTIRGVQYKNKSGQEMTAYAPLTIVCDGCFSNLRRSLCNPKVDVPSCFVGLILENCDLPHANHGHVILADPSPILFYPISSTEIRCLVDVPGQKVPSISNGEMANYLKNVVAPQIPPQLYNSFIAAIDKGNIRTMPNRSMPADPYPTPGALLMGDAFNMRHPLTGGGMTVALSDIVVLRDLLKPLRDLNDAPTLCKYLEAFYTLRKPVASTINTLAGALYKVFCASPDQARKEMRQACFDYLSLGGIFSNGPVSLLSGLNPRPLSLVLHFFAVAIYGVGRLLIPFPSPKRVWIGARLISGASAIIFPIIKAEGVRQMFFPKTVAAYYRAPPIVRER

SEQ ID NO: 53 - protein, 537 aa, synthetic construct
MVDQYALGLILASVLGFVVLYNLMAKKNRIRVSSEARTEGVQTVITTTNGECRSIEGDVDVIIVGAGVAGSALAHTLGKDGRKVHVIERDLSEPDRIVGELLQPGGYLKLVELGLQDSVEDIDAQRVFGYALFKDGKNTRLSYPLEKFHSDVSGRSFHNGRFIQRMREKAASLPNIRLEQGTVTSLLEENGTIKGVQYKTKDGKELAAHAPLTIVCDGCFSNLRRSLCNPQVDVPSSFVGLVLENCELPYANHGHVVLADPSPILFYPISSTEVRCLVDVPGQKVPSISNGEMAKYLKTMVAPQVPPEIYDSFVAAVDRGNIRTMPNRSMPAAPQPTPGALLMGDAFNMRHPLTGGGMTVALSDIVVLRDLLRPLRDLNDAPTLCKYLESFYTLRKPVASTINTLAGALYKVFCASPDRARNEMRQACFDYLSLGGVFSTGPVSLLSGLNPRPLSLVLHFFAVAVYGVGRLLVPFPSPSRMWIGARLISGASAIIFPIIKAEGVRQMFFPATVPAYYRAPPVNCQARSLKPDALKGL

SEQ ID NO: 54 - protein, 523 aa, synthetic construct
MADSYVWGWILGSVMTLVALCGVVLKRRKGSGISATRTESVKCVSSINGKCRSADGSDADVIIVGAGVAGSALAHTLGKDGRRVHVIERDLTEPDRIVGELLQPGGYLKLIELGLEDCVEEIDAQQVFGYALFKDGKHTRLSYPLEKFHSDVSGRSFHNGRFIQRMREKSASLPNVRLEQGTVTSLLEEKGTIRGVQYKTKDGRELTAFAPLTIVCDGCFSNLRRSLCNPKVDVPSCFVGLVLENCNLPYSNHGHVILADPSPILFYPISSTEVRCLVDVPGQKVPSIANGEMANYLKTIVAPQVPPEIYNSFVAAVDKGNIRTMPNRSMPAAPYPTPGALLMGDAFNMRHPLTGGGMTVALSDIVVLRDLLRPLRDLNDAPTLCKYLESFYTLRKPIASTINTLAGALYKVFCASPDQARKEMRQACFDYLSLGGVFSTGPISLLSGLNPRPVSLVLHFFAVAIYGVGRLLLPFPSPKRIWIGARLISGASGIIFPIIKAEGVRQMFFPATVPAYYRAPPVE

SEQ ID NO: 55 - protein, 531 aa, synthetic construct; identical to SEQ ID NO: 44

SEQ ID NO: 56 - protein, 525 aa, synthetic construct
MLDTYVFGWIICAALSVFVIRNFVFAGKKCCASSETDASMCAENITTAAGECRSSMRDGEFDVLIVGAGVAGSALAYTLGKDGRQVLVIERDLSEPDRIVGELLQPGGYLKLIELGLEDCVDKIDAQQVFGYALFKDGKHIRLSYPLEKFHSDVAGRSFHNGRFIQRMREKAASLPNVRLEQGTVTSLLEEKGVIKGVQYKTKDSQELSVCAPFTIVCDGCFSNLRRSLCDPKVDVPSCFVGLVLENCELPCANHGHVILGEPSPVLFYPISSTEIRCLVDVPGQKVPSISNGEMAKYLKTVIAPQVPHELHNAFIAAVDKGSIRTMPNRSMPAAPYPTPGALLMGDAFNMRHPLTGGGMTVALSDIVVLRNLLRPLRDLNDAPSLCKYLESFYTLRKPVASTINTLAGALYKVFCASSDPARKEMRQACFDYLSLGGQFSEGPISLLSGLNPRPLTLVLHFFAVATYGVGRLLLPFPSPKRMWIGLRLISSASGIIMPIIKAEGVRQMFFPATVPAYYRNPPAA

SEQ ID NO: 57 - protein, 533 aa, synthetic construct
MKMADHYLLGWILASVMGLFAFYYIVYLLVKPEEDNNRRSLPQPRSDFVKTMTATNGECRSDDDSDVDVIIVGAGVAGAALAHTLGKDGRRVHVIERDLTEPDRIVGELLQPGGYLKLIELGLEDCVEEIDAQRVFGYALFKDGKHTQLAYPLEKFHSEVAGRSFHNGRFIQRMREKAASLPSVKLEQGTVTSLLEEKGTIKGVLYKTKTGEELTAFAPLTIVCDGCFSNLRRSLCNPKVDVPSCFVGLVLENCRLPYANNGHVILADPSPILFYPISSTEVRSLVDVPGQKVPSVSSGEMANYLKNVVAPQVPPEIYDSFVAAVDKGNIRTMPNRSMPASPYPTPGALLMGDAFNMRHPLTGGGMTVALSDIVVLRDLLKPLRDLHDAPTLCRYLESFYTLRKPVASTINTLAGALYKVFCASPDEARKEMRQACFDYLSLGGVFSTGPVSLLSGLNPRPLSLVLHFFAVAIYGVGRLLLPFPSPHRIWVGARLISGASGIIFPIIKAEGVRQMFFPATVPAYYRAPPIKCN

SEQ ID NO: 58 - protein, 529 aa, synthetic construct
MAAAAAAASGVGFQLIGAAAATLLAAVLVAAVLGRRRRRARPQAPLVEAKPAPEGGCAVGDGRTDVIIVGAGVAGSALAYTLGKDGRRVHVIERDLTEPDRIVGELLQPGGYLKLIELGLEDCVEEIDAQRVLGYALFKDGRNTKLAYPLEKFHSDVAGRSFHNGRFIQRMRQKAASLPNVQLEQGTVTSLLEENGTVKGVQYKTKSGEELKAYAPLTIVCDGCFSNLRRALCSPKVDVPSCFVGLVLENCQLPHPNHGHVILANPSPILFYPISSTEVRCLVDVPGQKVPSIASGEMANYLKTVVAPQIPPEIYDSFIAAIDKGSIRTMPNRSMPAAPHPTPGALLMGDAFNMRHPLTGGGMTVALSDIVVLRNLLKPLHNLHDASSLCKYLESFYTLRKPVASTINTLAGALYKVFSASPDQARNEMRQACFDYLSLGGVFSNGPIALLSGLNPRPLSLVAHFFAVAIYGVGRLMLPLPSPKRMWIGARLISGACGIILPIIKAEGVRQMFFPATVPAYYRAAPMGE

SEQ ID NO: 59 - protein, 484 aa, synthetic construct
MRKNLEEAGCAVSDGGTDVIIVGAGVAGSALAYTLGKDGRRVHVIERDLTEPDRIVGELLQPGGYLKLIELGLQDCVEEIDAQRVLGYALFKDGRNTKLAYPLEKFHSDVAGRSFHNGRFIQRMRQKAASLPNVQLEQGTVTSLLEENGTVKGVQYKTKSGEELKAYAPLTIVCDGCFSNLRRALCSPKVDVPSCFVGLVLENCQLPHPNHGHVILANPSPILFYPISSTEVRCLVDVPGQKVPSIATGEMANYLKTVVAPQIPPEIYDSFIAAIDKGSIRTMPNRSMPAAPHPTPGALLMGDAFNMRHPLTGGGMTVALSDIVVLRNLLKPLRNLHDASSLCKYLESFYTLRKPVASTINTLAGALYKVFSASPDQARNEMRQACFDYLSLGGVFSNGPIALLSGLNPRPLSLVAHFFAVAIYGVGRLMLPLPSPKRMWIGARLISGACGIILPIIKAEGVRQMFFPATVPAYYRAAPTGEKA

SEQ ID NO: 60 - protein, 524 aa, synthetic construct
MDLYNIGWILSSVLSLFALYNLIFSGKRNYHDVNDKVKDSVTSTDAGDIQSEKLNGDADVIIVGAGIAGAALAHTLGKDGRRVHIIERDLSEPDRIVGELLQPGGYLKLVELGLQDCVDNIDAQRVFGYALFKDGKHTRLSYPLEKFHSDVSGRSFHNGRFIQRMREKAASLPNVNMEQGTVISLLEEKGTIKGVQYKNKDGQALTAYAPLTIVCDGCFSNLRRSLCNPKVDNPSCFVGLILENCELPCANHGHVILGDPSPILFYPISSTEIRCLVDVPGTKVPSISNGDMTKYLKTTVAPQVPPELYDAFIAAVDKGNIRTMPNRSMPADPRPTPGAVLMGDAFNMRHPLTGGGMTVALSDIVVLRNLLKPMRDLNDAPTLCKYLESFYTLRKPVASTINTLAGALYKVFSASPDEARKEMRQACFDYLSLGGLFSEGPISLLSGLNPRPLSLVLHFFAVAVFGVGRLLLPFPSPKRVWIGARLLSGASGIILPIIKAEGIRQMFFPATVPAYYRAPPVNAF

SEQ ID NO: 61 - protein, 458 aa, synthetic construct
MHTTSEHNDLFDICIVGAGMAGATIATYLAPRGIKIALIDRDYAEKRRIVGELLQPGAVQTLKKMGLEHLLEGFDAQPIYGYALFNKDCEFSIEYNQDKSTNYRGVGLHNGRFLQKIREDALKQPSITQIHGTVSELIEDENHVVTGVKYKEKYTRELKTVNAKLTITSDGFFSSFRKDLTNNVKTVTSFFVGIILKDCELPYPHHGHVFLSAPTPFICYPISSTESRLLIDFPGDQAPKKEAVKHHIENNVIPFLPKEFRLCLDQALRENDYKIMPNHYMPAKPVLKKGVVLLGDALNMRHPITGGGLTAVFNDVYLLSTHLLAMPDFNDTKLIHEKVNLYYNDRYHANTNVNIMANALYGVMSNDLLKQSVFEYLRKGGDNSGGPISLLAGLNRNPTILIKHFFSVALLCLRNLFKAHKMSLTNAFYVIKDAFCIIVPLAINELRPSSFLKKNIHN

SEQ ID NO: 62 - protein, 458 aa, synthetic construct
MNTSPEHNDLFDICIVGVGMAGATIAAYLAPRGLKIALIDREYTEKRRIVGELLQPGAVQTLKKMGLEHLLEGFDAQPIYGYALFNNDKEFSISYNSDDSTEYHGVGLHNGRFLQKIREDVFKNETVTQIHGTVSELIEDKKGVVKGVTYREKHTREYKTVKAKLTVTSDGFFSNFRKDLSNNVKTVTSFFIGLVLNDCNLPFPNHGHVFLSAPTPFICYPISSTETRLLIDYPGDKAPKKDEIREHILNKVAPFLPEEFKECFANAMEDDDFKVMPNHYMPAKPVLKEGAVLLGDALNMRHPLTGGGLTAVFNDVYLLSTHLLAMPDFNDPKLLHEKLELYYQDRYHANTNVNIMANALYGVMSNDLLKQGVFEYLRKGGDNSGGPITLLAGLNRNPTLLIKHFFSVAFLCICNLSGNNKMNFTNVFRVMKDAFCIIKPLAVNELRPSSFYKKNIQL

SEQ ID NO: 63 - protein, 448 aa, synthetic construct
MESNFDICIIGAGMAGATIAAYLAPKGINIALIDHCYKEKKRIVGELLQPGAVLSLEQLGLGHLLDGIDAQPVEGYALLQGNEQTTIPYPSPNHGMGLHNGRFLQQIRASALQNSSVTQIQGKALSLLENEQNEIIGVNYRDSVSNEIKSIYAPLTITSDGFFSNFRELLSNNEKTVTSYFIGLILKDCEIPVPKHGHVFLSGPTPFICYPISSNEVRLLIDFPGGQFPRKAFLQAHLETNVTPYIPEGMQTSYRHALQEDRLKVMPNHYMAAKPKIRKGAVMLGDALNMRHPLTGGGLTAVFSDIEILSGHLLAMPDFNNNDLIYQKIEAYYRDRQYANANLNILANALYGVMSNELLKNSVFKYLQRGGVNAKESIAILAGLNKNHYSLMKQFFFVALFGAYTLVRENITNLPKATKILSDALTIIKPLAKNELSLVGIFSDYFKR

SEQ ID NO: 64 - protein, 522 aa, synthetic construct
MVDPYAVGWIICSLTTIVALYNFVFYRQNRSDKTTPTTTENITTATGDCRSLNPNGDVDIVIVGAGVAGSALAYTLGKDGRRVLVIERDLNEPDRIVGELLQPGGYLKLIELGLEDCVEKIDAQQVFGYALFKDGKHTRLSYPLEKFHSDIAGRSFHNGRFIQRMREKAASLPNVQLVQGTVTSLLEENGTIKGVQYKTKDAQELSACAPLTIVCDGCFSNLRRNLCNPKVEVPSCFVGLVLENCELPCANHGHVILGDPSPVLFYPISSTEIRCLVDVPGQKVPSISNGEMAKYLKEVVAPQVPPELHDAFIAAVDKGNIRTMPNRSMPAAPYPTPGALLMGDAFNMRHPLTGGGMTVALSDIVVLRNLLKPLRDLNDAPSLCKYLESFYTLRKPVASTINTLAGALYKVFCASPDPARKEMRQACFDYLSLGGLFSEGPVSLLSGLNPRPLSLVLHFFAVAIYGVGRLLLPFPSPKRIWIGVRLIASASGIILPIIKAEGIRQMFFPATVPAYYRTPPAA

SEQ ID NO: 65 - protein, 525 aa, synthetic construct
MDLYLLGWILSSVLSLFALYCLVFDGNRSRANAEKQIQRGYSVTTDAGDVKSEKLNGDADVIIVGAGIAGAALAHTLGKDGRRVRVIERDLSEPDRIVGELLQPGGYLKLVELGLADCVDNIDAQKVFGYALFKDGKHTRLSYPLEKFHADVSGRSFHNGRFIQRMREKAASLLNVNLEQGTVTSLLEEKGTIKGVQYKNKDGQELTAYAPLTIVCDGCFSNLRRSLCNPKVDNPSCFVGLVLENCELPCANHGHVILGDPSPILFYPISSTEIRCLVDVPGQKVPSISNGDMTKYLKLTVAPQVPPELYDAFIAAVDKGNIRTMPNKSMPADPCPTPGAVLMGDAFNMRHPLTGGGMTVALSDIVVLRNLLRPLRDLNDAPALCKYLESFYTLRKPVASTINTLAGALYKVFSSSPDQARREMRQACFDYLSLGGLFSEGPISLLSGLNPRPLSLVLHFFAVAVFGVGRLLLPFPSPKRVWIGARLLSAASGIILPIIKAEGIRQMFFPVTVPAYYRAPPTSQE

SEQ ID NO: 66 - protein, 519 aa, synthetic construct
MIDPYGFGWITCTLITLAALYNFLFSRKNHSDSTTTENITTATGECRSFNPNGDVDIIIVGAGVAGSALAYTLGKDGRRVLIIERDLNEPDRIVGELLQPGGYLKLIELGLDDCVEKIDAQKVFGYALFKDGKHTRLSYPLEKFHSDIAGRSFHNGRFILRMREKAASLPNVRLEQGTVTSLLEENGTIKGVQYKTKDAQEFSACAPLTIVCDGCFSNLRRSLCNPKVEVPSCFVGLVLENCELPCADHGHVILGDPSPVLFYPISSTEIRCLVDVPGQKVPSISNGEMAKYLKTVVAPQVPPELHAAFIAAVDKGHIRTMPNRSMPADPYPTPGALLMGDAFNMRHPLTGGGMTVALSDIVVLRNLLKPLRDLNDASSLCKYLESFYTLRKPVASTINTLAGALYKVFCASPDPARKEMRQACFDYLSLGGLFSEGPVSLLSGLNPCPLSLVLHFFAVAIYGVGRLLLPFPSPKRLWIGIRLIASASGIILPIIKAEGIRQMFFPATVPAYYRAPPDA

SEQ ID NO: 67 - protein, 525 aa, synthetic construct
MDLYNIGWILSSVLSLFALYNLIFAGKKNYDVNEKVNQREDSVTSTDAGEIKSDKLNGDADVIIVGAGIAGAALAHTLGKDGRRVHIIERDLSEPDRIVGELLQPGGYLKLVELGLQDCVDNIDAQRVFGYALFKDGKHTRLSYPLEKFHSDVSGRSFHGRFIQRMREKAASLPNVNMEQGTVISLLEEKGTIKGVQYKNKDGQALTAYAPLTIVCDGCFSNLRRSLCNPKVDNPSCFVGLILENCELPCANHGHVILGDPSPILFYPISSTEIRCLVDVPGTKVPSISNGDMTKYLKTTVAPQVPPELYDAFIAAVDKGNIRTMPNRSMPADPRPTPGAVLMGDAFNMRHPLTGGGMTVALSDIVVLRNLLKPMRDLNDAPTLCKYLESFYTLRKPVASTINTLAGALYKVFSASPDEARKEMRQACFDYLSLGGLFSEGPISLLSGLNPRPLSLVLHFFAVAVFGVGRLLLPFPSPKRVWIGARLLSGASGIILPIIKAEGIRQMFFPATVPAYYRAPPVNAF

SEQ ID NO: 68 - protein, 461 aa, synthetic construct
MSKSRSNYDVIIVGAGIAGCALAHGLSTLSRATPLRIAIVERSLAEPDRIVGELLQPGGVMALQRLGMEGCLEGIDAVKVHGYCVVENGTSVHIPYPGVHEGRSFHHGRFIMKLREAARAARGVELVEATVTELIPREGGKGIAGVRVARKGKDGEEDTTEALGAALVVVADGCFSNFRAAVMGGAAVKPETKSHFVGAILKDARLPIPNHGTVALVKGFGPVLLYQISEHDTRMLVDVKAPLPADLKVCAHILSNIVPQLPAALHLPIQRALDAERLRRMPNSFLPPVEQGATRGAVLVGDAWNMRHPLTGGGMTVALNDVVVLRDLLGSVGDLGDWRQVASTVNILSVALYDLFGADGELQVLRTGCFKYFERGGDCIDGPVSLLSGIAPSPMLLAYHFFSVAFYSIYVIAVGAQNGSAKQVLAVPGALQYPALCVKGLRVFYTACVVFGPLLWTELRW

SEQ ID NO: 69 - protein, 498 aa, synthetic construct
MHPTHYDVVIVGAGVAGSSLAHALATLPREKPLQIALIERSFEEPDRIVGELLQPGGVDALKTLKMTSSVEGIDAITVTGYILVESGDMVRIPYPKGKEGRSFHHGRFIMGLRRVALENPNVHPIEATAADLIECPCTGQVIGVRATSKTAPAPSSIDAQQTPPAPFSVYGDLVIVADGCFSNFRNVVMGKAACKATTKSYFVGTILKDAVLPVAGHGTVILPQGSGPVLLYQISEHDTRMLIDIQHPLPSDLRAHILTNILPQLPASIQGVVSDAFTKDRIRRMPNSFLPSVQQGSPLSKKGVILLGDSWNMRHPLTGGGMTVALNDVVYLRSIFASIQNLDDWDEIRYALRHWHWGRKPLSSTINILSGTLYGLFEKDDDDYRALRKGCFKYFQLGGKCIDDPVSLLSGLSPSPLLLSSHFFAVILYAIWVVFTHPRVGSSMSANPADVKRVYDIPSADEYPQLTLKGIRMFSQACGVFLPVLWSEIRWWAPCESS

SEQ ID NO: 70 - protein, 524 aa, synthetic construct
MSKSRSNYDVIIVGAGIAGCALAHGLSTLSRATPLRIAIVERSLAEPDRIVGELLQPGGVMALQRLGMEGCLEGIDAVKVHGYCVVENGTSVHIPYPGVHEGRSFHHGRFIMKLREAARAARGVELVEATVTELIPREGGKGIAGVRVARKGKDGEEDTTEALGAALVVVADGCFSNFRAAVMGGAAVKPETKSHFVGAILKDARLPIPNHGTVALVKGFGPVLLYQISEHDTRMLVDVKAPLPADLKAHILSNIVPQLPAALHLPIQRALDAERLRRMPNSFLPPVEQGATRGAVLVGDAWNMRHPLTGGGMTVALNDVVVLRDLLGSVGDLGDWRQVRRALHRWHWDRKPLASTVNILSVALYDLFGADGEELQVLRTGCFKYFERGGDCIDGPVSLLSGIAPSPMLLAYHFFSVAFYSIYVMFAHPQPVAQSKAVGAQNGSAKQVLAVPGALQYPALCVKGLRVFYTACVVFGPLLWTELRWWTAAEASRGRLLVMSLVPLLLLLGAANYGIPGMGLLGVL

SEQ ID NO: 71 - protein, 711 aa, synthetic construct
MAQSDSVKVSPFDLVSAAMNGKAMEKLNASESEDPTTLPALKMLVENRELLTLFTTSFAVLIGCLVFLMWRRSSSKKLVQDPVPQVIVVKKKEKESEVDDGKKKVSIFYGTQTGTAEGFAKALVEEAKVRYEKTSFKVIDLDDYAADDDEYEEKLKKESLAFFFLATYGDGEPTDNAANFYKWFTEGDDKGEWLKKLQYGVFGLGNRQYEHFNKIAIVVDDKLTEMGAKRLVPVGLGDDDQCIEDDFTAWKELVWPELDQLLRDEDDTSVTTPYTAAVLEYRVVYHDKPADSYAEDQTHTNGHVVHDAQHPSRSNVAFKKELHTSQSDRSCTHLEFDISHTGLSYETGDHVGVYSENLSEVVDEALKLLGLSPDTYFSVHADKEDGTPIGGASLPPPFPPCTLRDALTRYADVLSSPKKVALLALAAHASDPSEADRLKFLASPAGKDEYAQWIVANQRSLLEVMQSFPSAKPPLGVFFAAVAPRLQPRYYSISSSPKMSPNRIHVTCALVYETTPAGRIHRGLCSTWMKNAVPLTESPDCSQASIFVRTSNFRLPVDPKVPVIMIGPGTGLAPFRGFLQERLALKESGTELGSSIFFFGCRNRKVDFIYEDELNNFVETGALSELIVAFSREGTAKEYVQHKMSQKASDIWKLLSEGAYLYVCGDAKGMAKDVHRTLHTIVQEQGSLDSSKAELYVKNLQMSGRYLRDVW

SEQ ID NO: 72 - protein, 693 aa, synthetic construct
MATSALYASDLFKQLKSIMGTDSLSDDVVLVIATTSLALVAGFVVLLWKKTTADRSGELKPLMIPKSLMAKDEDDDLDLGSGKTRVSIFFGTQTGTAEGFAKALSEEIKARYEKAAVKVIDLDDYAADDDQYEEKLKKETLAFFCVATYGDGEPTDNAARFYKWFTEENERDIKLQQLAYGVFALGNRQYEHFNKIGIVLDEELCKKGAKRLIEVGLGDDDQSIEDDFNAWKESLWSELDKLLKDEDDKSVATPYTAVIPEYRVVTHDPRFTTQKSMESNVANGNTTIDIHHPCRVDVAVQKELHTHESDRSCIHLEFDISRTGITYETGDHVGVYAENHVEIVEEAGKLLGHSLDLVFSIHADKEDGSPLESAVPPPFPGPCTLGTGLARYADLLNPPRKSALVALAAYATEPSEAEKLKHLTSPDGKDEYSQWIVASQRSLLEVMAAFPSAKPPLGVFFAAIAPRLQPRYYSISSSPRLAPSRVHVTSALVYGPTPTGRIHKGVCSTWMKNAVPAEKSHECSGAPIFIRASNFKLPSNPSTPIVMVGPGTGLAPFRGFLQERMALKEDGEELGSSLLFFGCRNRQMDFIYEDELNNFVDQGVISELIMAFSREGAQKEYVQHKMMEKAAQVWDLIKEEGYLYVCGDAKGMARDVHRTLHTIVQEQEGVSSSEAEAIVKKLQTEGRYLRDVW

SEQ ID NO: 73 - protein, 712 aa, synthetic construct
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 74 - protein, 713 aa, synthetic construct
MASSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDITLANGNGYTVFDAQHPYKANVAVKRELHTPESDRSCIHLEFDIAGSGLTMKLGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSEKLFLGRPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 75 - protein, 710 aa, synthetic construct
MAQSESVEASTIDLMTAVLKDTVIDTANASDNGDSKMPPALAMMFEIRDLLLILTTSVAVLVGCFVVLVWKRSSGKKSGKELEPPKIVVPKRRLEQEVDDGKKKVTIFFGTQTGTAEGFAKALFEEAKARYEKAAFKVIDLDDYAADLDEYAEKLKKETYAFFFLATYGDGEPTDNAAKFYKWFTEGDEKGVWLQKLQYGVFGLGNRQYEHFNKIGIVVDDGLTEQGAKRIVPVGLGDDDQSIEDDFSAWKELVWPELDLLLRDEDDKAAATPYTAAIPEYRVVFHDKPDAFSDDHTQTNGHAVHDAQHPCRSNVAVKKELHTPESDRSCTHLEFDISHTGLSYETGDHVGVYCENLIEVVEEAGKLLGLSTDTYFSLHIDNEDGSPLGGPSLQPPFPPCTLRKALTNYADLLSSPKKSTLLALAAHASDPTEADRLRFLASREGKDEYAEWVVANQRSLLEVMEAFPSARPPLGVFFAAVAPRLQPRYYSISSSPKMEPNRIHVTCALVYEKTPAGRIHKGICSTWMKNAVPLTESQDCSWAPIFVRTSNFRLPIDPKVPVIMIGPGTGLAPFRGFLQERLALKESGTELGSSILFFGCRNRKVDYIYENELNNFVENGALSELDVAFSRDGPTKEYVQHKMTQKASEIWNMLSEGAYLYVCGDAKGMAKDVHRTLHTIVQEQGSLDSSKAELYVKNLQMSGRYLRDVW

SEQ ID NO: 76 - protein, 708 aa, synthetic construct
MAQSNSVKISPLDLVTALFSGKVLDTSNASESGESAMLPTIAMIMENRELLMILTTSVAVLIGCVVVLVWRRSSTKKSALEPPVIVVPKRVQEEEVDDGKKKVTVFFGTQTGTAEGFAKALVEEAKARYEKAVFKVIDLDDYAADDDEYEEKLKKESLAFFFLATYGDGEPTDNAARFYKWFTEGDAKGEWLNKLQYGVFGLGNRQYEHFNKIAKVVDDGLVEQGAKRLVPVGLGDDDQCIEDDFTAWKELVWPELDQLLRDEDDTTVATPYTAAVAEYRVVFHEKPDALSEDYSYTNGHAVHDAQHPCRSNVAVKKELHSPESDRSCTHLEFDISNTGLSYETGDHVGVYCENLSEVVNDAERLVGLPPDTYFSIHTDSEDGSPLGGASLPPPFPPCTLRKALTCYADVLSSPKKSALLALAAHATDPSEADRLKFLASPAGKDEYSQWIVASQRSLLEVMEAFPSAKPSLGVFFASVAPRLQPRYYSISSSPKMAPDRIHVTCALVYEKTPAGRIHKGVCSTWMKNAVPMTESQDCSWAPIYVRTSNFRLPSDPKVPVIMIGPGTGLAPFRGFLQERLALKEAGTDLGLSILFFGCRNRKVDFIYENELNNFVETGALSELIVAFSREGPTKEYVQHKMSEKASDIWNLLSEGAYLYVCGDAKGMAKDVHRTLHTIVQEQGSLDSSKAELYVKNLQMSGRYLRDVW

SEQ ID NO: 77 - protein, 705 aa, synthetic construct
MAQSTTSVKLSPFDLMTALLNGKVSFDTSNTSDTNIPLAVFMENRELLMILTTSVAVLIGCVVVLVWRRSSSAAKKAAESPVIVVPKKVTEDEVDDGRKKVTVFFGTQTGTAEGFAKALVEEAKARYEKAVFKVIDLDDYAAEDDEYEEKLKKESLAFFFLATYGDGEPTDNAARFYKWFTEGEEKGEWLDKLQYAVFGLGNRQYEHFNKIAKVVDEKLVEQGAKRLVPVGMGDDDQCIEDDFTAWKELVWPELDQLLRDEDDTSVATPYTAAVAEYRVVFHDKPETYDQDQLTNGHAVHDAQHPCRSNVAVKKELHSPLSDRSCTHLEFDISNTGLSYETGDHVGVYVENLSEVVDEAEKLIGLPPHTYFSVHADNEDGTPLGGASLPPPFPPCTLRKALASYADVLSSPKKSALLALAAHATDSTEADRLKFLASPAGKDEYAQWIVASHRSLLEVMEAFPSAKPPLGVFFASVAPRLQPRYYSISSSPRFAPNRIHVTCALVYEQTPSGRVHKGVCSTWMKNAVPMTESQDCSWAPIYVRTSNFRLPSDPKVPVIMIGPGTGLAPFRGFLQERLAQKEAGTELGTAILFFGCRNRKVDFIYEDELNNFVETGALSELVTAFSREGATKEYVQHKMTQKASDIWNLLSEGAYLYVCGDAKGMAKDVHRTLHTIVQEQGSLDSSKAELYVKNLQMAGRYLRDVW

SEQ ID NO: 78 - protein, 706 aa, synthetic construct
MAQSSSGSMSPFDFMTAIIKGKMEPSNASLGAAGEVTAMILDNRELVMILTTSIAVLIGCVVVFIWRRSSSQTPTAVQPLKPLLAKETESEVDDGKQKVTIFFGTQTGTAEGFAKALADEAKARYDKVTFKVVDLDDYAADDEEYEEKLKKETLAFFFLATYGDGEPTDNAARFYKWFLEGKERGEWLQNLKFGVFGLGNRQYEHFNKIAIVVDEILAEQGGKRLISVGLGDDDQCIEDDFTAWRESLWPELDQLLRDEDDTTVSTPYTAAVLEYRVVFHDPADAPTLEKSYSNANGHSVVDAQHPLRANVAVRRELHTPASDRSCTHLEFDISGTGIAYETGDHVGVYCENLAETVEEALELLGLSPDTYFSVHADKEDGTPLSGSSLPPPFPPCTLRTALTLHADLLSSPKKSALLALAAHASDPTEADRLRHLASPAGKDEYAQWIVASQRSLLEVMAEFPSAKPPLGVFFASVAPRLQPRYYSISSSPRIAPSRIHVTCALVYEKTPTGRVHKGVCSTWMKNSVPSEKSDECSWAPIFVRQSNFKLPADAKVPIIMIGPGTGLAPFRGFLQERLALKEAGTELGPSILFFGCRNSKMDYIYEDELDNFVQNGALSELVLAFSREGPTKEYVQHKMMEKASDIWNLISQGAYLYVCGDAKGMARDVHRTLHTIAQEQGSLDSSKAESMVKNLQMSGRYLRDVW

SEQ ID NO: 79 - protein, 709 aa, synthetic construct; identical to SEQ ID NO: 22

SEQ ID NO: 80 - protein, 352 aa, synthetic construct
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 81 - protein, 770 aa, synthetic construct
MAWKLKVAQGNDPYLYSTNNFVGRQYWEFDPDAGTPEEREEVEKARKDYVNNKKLHGIHPCSDLLMRMQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGAEETRYITTGNGEKALQIMSWWAEDPNGDEFKHHLARIPDFLWIAEDGMTVQSFGSQLWDCALATQAIIATNMVEEYGDSLKKAHFFIKESQIKENPSGDFKKMYRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAILGLMFGGQAERDPTPLHRAAKLLINAQMDNGDFPQQEITGVYCKNSMLHYAEYRNIFPLWALGEYRKRVWLPKHQQLKI

SEQ ID NO: 82 - protein, 767 aa, synthetic construct
MAWKLKVAQGNDPYLYSTNNFVGRQYWEFDPDAGTPEEREEVEKARKDFVNNKKLHGIHPCSDLLMRMQLIKESGIDLLSIPPVRLDENEQVNYDAVTTAVKKALRLNRAIQAHDGHWPAENAGSLLYTPPLIIALYISGTIDTILTKQHKKELIRFVYNHQNEDGGWGSYIEGHSTMIGTVLSYVMLRLLGEGPDDGNGAVERGRKWILDHGGAAGIPSWGKTYLAVLGVYEWSGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGAEETRYITTGNGEKALQIMSWWAEDPNGDEFKHHLARIPDFLWVAEDGMTVQSFGSQLWDCALATQAIIATNMVEEYGDSLKKAHFFIKESQIKENPSGDFKKMYRQFTKGAWTFSDQDHGCVVSDCTAEALKCLLLLSQMPQEIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGKTYDNSEAVRKGVKFFLSTQNEEGGWGESLESCPSEKFTPLKGNRTNLVQTSWAILGLIFGGQAERDPTPLHRAAKLLINAQMDNGDFPQQEITGVYCKNSMLHYAEYRNIFPLWALGEYRKRVWLPKHQQLKI

SEQ ID NO: 83 - protein, 487 aa, synthetic construct
MAQDLRLILIIVGAIAIIALLVHGFWSYTKRIPQKENDSKAPLPPGQTGWPLIGETLEYLSCVKSGVPENFVKYRKEKYSPKVFRTSLLGEPMAILCGPEGNKFLYSNEKKLVQVWFPSSVEKMFPRSHGESNAENFSKVRGKMMFLLKPDGMKKYVGLMDRVMKQHLETDWNRQQQINVHNTVKKYTVTMSCRVFMSIDDEEQVTRLGSSFQNIEAGLLAVPINIPGTAMNRAIKTVKLLTKEVEAVIKQRKVDLLENKQASQPQDLLSHLLLTANEDGQFMSESDIASHIIGLMQGGYTTLNGTITFVLNYLAEFPDVYNQVLKEQMEIANSKHPGELLNWEDLQKMKYSWNVAQEVLRIIPPGVGTFREAITDFTYAGYLIPKGWKLHLIPHDTHKNPTYFPSPEKFDPTRFEGNGPAPYTFTPFGGGPRMCPGIEYARLVILIFMHNVVTNFRWEKLIPNEKILTDPIPRFAHGLPIRLHPHN

SEQ ID NO: 84 - protein, 474 aa, synthetic construct
MAQDLRLILIIVGAIAIIALLVHGFWNFKPSSQNKLPPGKTGWPIIGETLEFISCGQKGNPEKFVTQRMNKYSPDVFTTSLAGEKMVVFCGASGNKFIFSNENKLVVSWWPPAISKILTATIPSVEKSKALRSLIVEFLKPEALHKFISVMDRTTRQHFEDKWNGKTEVKAFAMSESLTFELACWLLFSINDPVQVQKLSHLFEKVKAGLLSLPLNFPGTAFNRGIKAANLIRKELSVIIKQRRSDKLQPRQDLLSHVMLSNGEGEKFFSEMDIADVVLNLLIASHDTTSSAMTSVVYFLADHPHIYAKVLTEQMEIAKSKGPEELLSWEDIKRMKYSRNVINEAMRLVPPSQGGFKVVTSDFSYANFTIPKGWKIFWSVYSTHKDPKYFKNPEEFDPSRFEGDGPMPFTFVPFGGGPRMCPGSEFARLEVLIFMHHLVTNFKWEKVFPNEKIIYTPFPFPENGLPIRLSPHTL

SEQ ID NO: 85 - protein, 767 aa, synthetic construct
MAWKLKVAQGNDPYLYSTNNFVGRQYWEFDPDAGTPEEREEVEKARKDFVNNKKLHGIHPCSDLLMRMQLIKESGIDLLSIPPVRLDENEQVNYDAVTTAVKKALRLNRAIQAHDGHWPSENAGSLLYTPPLIIALYISGTIDTILTKEHKKELIRFVYNHQNEDGGWGSYIEGHSTMIGTVLSYVMLRLLGEGPDDGNGAVERGRKWILDHGGAAGIPSWGKTYLAVLGVYEWSGCNPLPPEFWLFPSSFPFHPGKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGAEETRYITTGNGEKALQIMAWWAEDPNGDEFKHHLARIPDFLWVAEDGMTVQSFGSQLWDCALATQAIIATNMVEEYGDSLKKAHFFIKESQIKENPSGDFKKMYRQFTKGAWTFSDQDHGCVVSDCTAEALKCLLLLSQMPQEIVGEKPEVERLYEAVNVLLYLQSRVSGGFAVWEPPVPKPYLEMFNPSEIFADIVVEREHIECTASVIKALMAFKCLHPGHRQKEIEDSVAKAIRYLERNQMPDGSWYGFWGICFLYGTFFTLSGFASAGKTYDNSEAVRKGVKFLLSTQNEEGGWGESLESCPSEKFTPLKGNRTNLVQTSWAILGLIFGGQAERDPTPLHRAAKLLINAQMDNGDFPQQEITGVYCKNSMLHYAEYRNIFPLWALGEYRKRVWLPKHQQLKI

(19) United States
(12) Patent Application Publication
(10) Pub. No.: US 2024/0392340 A1
(43) Pub. Date: Nov. 28, 2024

(54) ENZYMES, HOST CELLS, AND METHODS FOR BIOSYNTHESIS OF DAMMARENEDIOL AND DERIVATIVES

(71) Applicant: Manus Bio Inc., Cambridge, MA (US)

(72) Inventors: Ajikumar PARAYIL KUMARAN, Waltham, MA (US); Christine Nicole S. SANTOS, Waltham, MA (US); Lu CHEN, Waltham, MA (US); Liwei LI, Waltham, MA (US); Chin Giaw LIM, Waltham, MA (US)

(21) Appl. No.: 18/690,805

(22) PCT Filed: Sep. 9, 2022
(86) PCT No.: PCT/US22/76194

§ 371 Date: Mar. 11, 2024

Related U.S. Application Data
(60) Provisional application No. 63/242,212, filed on Sep. 9, 2021.

(51) Int. Cl. C12P 33/06; C12N 9/02; C12N 9/10; C12N 9/88; C12N 15/52; C12N 15/70

(52) U.S. Cl. CPC C12P 33/06; C12N 9/0071; C12N 9/1051; C12N 9/1085; C12N 9/88; C12N 15/52; C12N 15/70; C12Y 114/14; C12Y 204/01017; C12Y 205/01021; C12Y 402/01125

(57) ABSTRACT

The disclosure provides compositions and methods related to engineered microbial cells, enzymes, and methods for producing dammarenediol, as well as compounds derived from dammarenediol. Microbial host cells are engineered to express a heterologous biosynthetic pathway that produces dammarenediol, or a derivative thereof. The host cell can optionally express a heterologous uridine diphosphate-dependent glycosyltransferase (UGT) enzyme producing natural or non-natural glycosylated forms of dammarenediol, protopanaxadiol or protopanaxatriol.

### BACKGROUND

Protopanaxatriol (C₃₀H₅₂O₄) is a triterpenoid with a dammarane-type tetracyclic scaffold that is derived from Dammarenediol-II (“dammarenediol”). Protopanaxatriol can be used as a pesticide, for example, against sucking and chewing insects. In addition, protopanaxadiol and protopanaxatriol are known to have various biological properties, including anti-inflammatory, anxiolytic, anti-stress, and anti-tumor effects. Oh et al., Anti-stress effects of 20(S)-protopanaxadiol and 20(S)-protopanaxatriol in immobilized mice, Biol Pharm Bull 38(2):331-335 (2015). Protopanaxatriol, which naturally occurs in Panax ginseng (ginseng) and Panax pseudoginseng (notoginseng), is conventionally isolated from ginseng root. Natural ginseng is very rare. Cultivated ginseng is very slow growing, requiring about seven to eleven years of growth before harvest. Therefore, there is a need for biotechnology processes that produce dammarenediol and its derivatives, including protopanaxadiol, protopanaxatriol, and including glycosylated forms or ginsenosides. Ginsenosides have a long history of use to boost immunity, and have a wide spectrum of pharmacological activities. Zhou et al., The Synergistic Effects of Polysaccharides and Ginsenosides From American Ginseng (Panax quinquefolius L.) Ameliorating Cyclophosphamide-Induced Intestinal Immune Disorders and Gut Barrier Dysfunctions Based on Microbiome-Metabolomics Analysis, Frontiers in Immunology 12: 1273 (2021); Leung and Wong Pharmacology of ginsenosides: a literature review. Chin Med 5: 1-7 (2010).

### SUMMARY OF THE DISCLOSURE

In accordance with various embodiments, the invention provides engineered microbial cells, enzymes, and methods for producing dammarenediol-II (“dammarenediol”) as well as compounds derived from dammarenediol, such as but not limited to protopanaxadiol and protopanaxatriol, and glycosylated forms thereof (“e.g., ginsenosides”). In accordance with the disclosure, microbial host cells are engineered to express a heterologous biosynthetic pathway that produces dammarenediol (or a derivative thereof). The heterologous pathway will generally comprise a dammarenediol synthase (DDS) enzyme (such as an engineered DDS described herein) which acts on 2,3-oxidosqualene substrate, and in various embodiments further comprises a protopanaxadiol synthase (PPDS) enzyme for production of protopanaxadiol (which can be an engineered PPDS described herein), and optionally a protopanaxatriol synthase (PPTS) enzyme for production of protopanaxatriol (which can be an engineered PPTS described herein). In some embodiments, the host cell can further express a heterologous uridine diphosphate-dependent glycosyltransferase (UGT) enzyme producing natural or non-natural glycosylated forms of dammarenediol, protopanaxadiol or protopanaxatriol, generally referred to as ginsenosides.

Accordingly, in various aspects, the present disclosure provides microbial host cells and methods for producing dammarenediol or a derivative thereof (i.e., a compound derived from dammarenediol), and which involves expressing a heterologous biosynthetic pathway.

The heterologous biosynthetic pathway comprises one or more of: a dammarenediol synthase (DDS) enzyme comprising an amino acid sequence that has at least about 70% sequence identity to the amino acid sequence of SEQ ID NO: 3, SEQ ID NO: 4, or SEQ ID NO: 9; a protopanaxadiol synthase (PPDS) enzyme comprising an amino acid sequence that has at least about 70% sequence identity to the amino acid sequence of SEQ ID NO: 10, SEQ ID NO: 11, SEQ ID NO: 12, SEQ ID NO: 13, SEQ ID NO: 14, SEQ ID NO: 15, or SEQ ID NO: 16; and a protopanaxatriol synthase (PPTS) enzyme comprising an amino acid sequence that has at least about 70% sequence identity to the amino acid sequence of SEQ ID NO: 17, SEQ ID NO: 18, SEQ ID NO: 19, SEQ ID NO: 20, and SEQ ID NO: 21.

In some embodiments, the DDS enzyme comprises an amino acid sequence having at least 70% sequence identity to SEQ ID NO: 3. Exemplary engineered derivatives of SEQ ID NO: 3 include enzymes having one or more mutations shown in Tables 1 and 2, and the enzymes represented by SEQ ID NOS: 81, 82, and 85.

In various aspects the heterologous biosynthetic pathway comprises a PPDS enzyme comprising an amino acid sequence that has at least 70% sequence identity to the amino acid sequence of SEQ ID NO: 10. Exemplary engineered PPDS enzymes of the disclosure can have one or more mutations shown in Table 3. In exemplary embodiments, the PPDS enzyme comprises at least one, two, three, four, five, six, seven, eight, or all of the following mutations with respect to SEQ ID NO: 10: T108N, I212F, K338G, D135E, S68P, V150P, F167H, L283M, H482R, R347Q, M390L, R243K, L292I, V329M, Q278E, and N58E. An exemplary engineered derivative of SEQ ID NO: 10 includes enzymes comprising the amino acid sequence of SEQ ID NO: 83.

In various aspects, the heterologous biosynthetic pathway comprises a PPTS enzyme comprising an amino acid sequence that has at least about 70% sequence identity to the amino acid sequence of SEQ ID NO: 17. Exemplary engineered PPTS enzymes of the disclosure include enzymes having one or more mutations shown in Table 4. In some embodiments, the PPTS enzyme comprises at least 2, or at least 3, or at least 4, or at least 5, or at least 8, or at least 10 amino acid substitutions with respect to SEQ ID NO: 17 as listed in Table 4. Exemplary PPTS enzymes comprise at least two, at least 3, at least 4, at least 5, or all amino acid substitutions, with respect to SEQ ID NO: 17, selected from: G294T, S166K, C472H, K252Q, V239I, A323P, I412V, I369T, K362D, and T250P. An exemplary engineered derivative of SEQ ID NO: 17 includes enzymes comprising the amino acid sequence of SEQ ID NO: 84.

In some embodiments, the heterologous enzyme pathway further comprises one or more uridine diphosphate-dependent glycosyltransferase (UGT) enzymes, thereby producing one or more glycosides of dammarenediol, protopanaxadiol and/or protopanaxatriol.

In some embodiments, the heterologous biosynthetic pathway further comprises a squalene synthase (SQS) enzyme as well as a squalene monooxygenase (SQE) producing 2,3-oxidosqualene from farnesyl diphosphate. The DDS enzyme acts on the 2,3-oxidosqualene substrate to produce the cyclic dammarenediol product.

In some embodiments, the biosynthetic pathway is expressed in a microbial host cell (such as a bacterium or yeast) that expresses an enzymatic pathway that produces iso-pentenyl diphosphate (IPP) and dimethylallyl diphosphate (DMAPP). IPP and DMAPP substrates are used to produce farnesyl diphosphate. In some embodiments, the enzymatic pathway is a methylerythritol phosphate (MEP) pathway and/or a mevalonic acid (MVA) pathway.

In various embodiments, the microbial host cells are further suitable for production at commercial scale, which can include culturing in batch culture, continuous culture, or semi-continuous culture.

In still other aspects, the invention provides engineered DDS, PPDS, and PPTS enzymes providing improved productivities or stability in host cells, including for microbial production of dammarenediol, protopanaxadiol, and protopanaxatriol, and glycosylated forms thereof.

The dammarenediol, protopanaxadiol, protopanaxatriol, or ginsenoside derived therefrom obtained according to this disclosure may be incorporated into pesticide or insecticide composition. In some embodiments, the product is incorporated into a pharmaceutical composition for use as an active pharmaceutical agent having anti-inflammatory, anxiolytic, anti-stress, and anti-tumor activity. In some embodiments, the product is incorporate into food products (including beverages) and nutraceutical products.

Other aspects and embodiments of this disclosure will be apparent from the following Drawings and Detailed Description.

### BRIEF DESCRIPTION OF THE DRAWINGS

FIG. 1 shows a schematic representation of the biosynthetic pathway of dammarenediol-type ginsenosides in Panax ginseng.

FIG. 2 shows the biosynthetic pathway engineered in microbial cells for the production of protopanaxatriol. IPP (as a product of the MVA or MEP pathway) is converted to farnesyl diphosphate (FPP), squalene, and 2,3-oxidosqualene in consecutive reactions. 2,3-oxidosqualene is converted to dammarenediol by cyclization via the action of dammarenediol synthase (DDS). Consecutive hydroxylations generate protopanaxadiol and protopanaxatriol, respectively.

FIG. 3A to FIG. 3C show the production of squalene, oxidosqualene and dammarenediol by engineered bacterial cells. Relative titers of squalene, oxidosqualene and dammarenediol are plotted for strains expressing SQS, SQE, and DDS in a base E. coli strain that produces farnesyl diphosphate. FIG. 3A shows the relative titers of squalene, oxidosqualene and dammarenediol produced by strains expressing SQS1 (SEQ ID NO: 1) and SQE1 (SEQ ID NO: 2) only (left); and strains further expressing DDS1 (SEQ ID NO: 3) (middle) or DDS7 (SEQ ID NO: 9) (right). FIG. 3B shows the relative titers of squalene, oxidosqualene and dammarenediol produced by E. coli strains expressing SQS1 (SEQ ID NO: 1), SQE1 (SEQ ID NO: 2), and DDS1 (SEQ ID NO: 3) (right) or DDS2 (SEQ ID NO: 4) (left). FIG. 3C shows the relative titers of squalene, oxidosqualene and dammarenediol produced by E. coli strains expressing SQS1 (SEQ ID NO: 1), SQE1 (SEQ ID NO: 2), and either DDS1 (SEQ ID NO: 3) or an engineered DDS1 derivative that is engineered to improve stability in E. coli (one of DDS3 to DDS6, (SEQ ID NOs: 5-8)).

FIG. 3D to 3F compare the production (relative titers) of dammarenediol by various DDS derivatives (Pq.DDS1, SEQ ID NO: 81) and derivatives (Pq.DDS2, SEQ ID NO: 82; and Pq.DDS3). FIG. 3D shows the relative titer of dammarenediol produced by strains expressing SQS1 (SEQ ID NO: 1), SQE1 (SEQ ID NO: 2), and engineered DDS1 derivative Pq.DDS1. Strain expressing SQS1, SQE1, and DDS5 is shown as a comparison. FIG. 3E shows the relative titer of dammarenediol produced by strains expressing SQS1, SQE1, and Pq.DDS2. Strain expressing SQS1, SQE1, and Pq.DDS1 is shown as a comparison. FIG. 3F shows the relative titer of dammarenediol produced by strains expressing SQS1, SQE1, and Pq.DDS3. Strain expressing SQS1, SQE1, and Pq.DDS2 is shown as a comparison.

FIG. 4A-B shows the production of protopanaxadiol in E. coli. FIG. 4A: Each of protopanaxadiol synthases PPDS1 to PPDS7 (each with a membrane anchor) (SEQ ID NOs: 10-16) were expressed in E. coli producing dammarenediol, along with cytochrome P450 reductase partner (CPR1, SEQ ID NO: 22). The strains were incubated at 30° C. for 72 hr. Dammarenediol and protopanaxadiol were quantified by GC-FID chromatography using authentic standards of each compound. Relative titers of dammarenediol and protopanaxadiol are shown. Productions of protopanaxadiol were verified by GC-MS spectrum analysis. FIG. 4B: The relative titer of protopanaxadiol is shown with strains expressing SQS1, SQE1, Pq.DDS3, CPR1, and Pq.PPDS1. The relative titer with strains expressing SQS1, SQE1, Pq.DDS3, CPR1, and PPDS1 is shown as a comparison.

FIG. 5A-B shows the production (relative titer) of protopanaxatriol in E. coli by co-expressing a protopanaxatriol synthase (PPTS). FIG. 5A: The following enzymes were expressed in a strain producing dammarenediol: (left) PPDS1 (SEQ ID NO: 10), PPTS1 (SEQ ID NO: 17) and CPR1 (SEQ ID NO: 22), or (right) PPDS1 (SEQ ID NO: 10), PPTS2 (SEQ ID NO: 18) and CPR1 (SEQ ID NO: 22). The strains were incubated at 30° C. for 72 hr. Dammarenediol, protopanaxadiol and protopanaxatriol were quantified by GC-FID chromatography using authentic standards of each compound. The relative titers of dammarenediol, protopanaxadiol and protopanaxatriol are shown. Productions of protopanaxatriol were verified by GC-MS spectrum analysis. FIG. 5B: The relative titer of protopanaxatriol is shown produced by strains expressing SQS1, SQE1, Pq.DDS3, CPR1, Pg.PPDS1, and Pg.PPTS2. The relative titer with strains expressing SQS1, SQE1, Pq.DDS3, CPR1, PPDS1, and PPTS1 is shown as a comparison.

### DETAILED DESCRIPTION

In accordance with various embodiments, the invention provides engineered microbial cells, enzymes, and methods for producing dammarenediol-II (“dammarenediol”) as well as compounds derived from dammarenediol, such as but not limited to protopanaxadiol and protopanaxatriol, and glycosylated forms thereof (“e.g., ginsenosides”). In accordance with the disclosure, microbial host cells are engineered to express a heterologous biosynthetic pathway that produces dammarenediol (or a derivative thereof). The heterologous pathway will generally comprise a dammarenediol synthase (DDS) enzyme (such as an engineered DDS described herein) which acts on 2,3-oxidosqualene substrate, and in various embodiments further comprises a protopanaxadiol synthase (PPDS) enzyme for production of protopanaxadiol (which can be an engineered PPDS described herein), and optionally a protopanaxatriol synthase (PPTS) enzyme for production of protopanaxatriol (which can be an engineered PPTS described herein). In some embodiments, the host cell can further express a heterologous uridine diphosphate-dependent glycosyltransferase (UGT) enzyme producing natural or non-natural glycosylated forms of dammarenediol, protopanaxadiol or protopanaxatriol, generally referred to as ginsenosides.

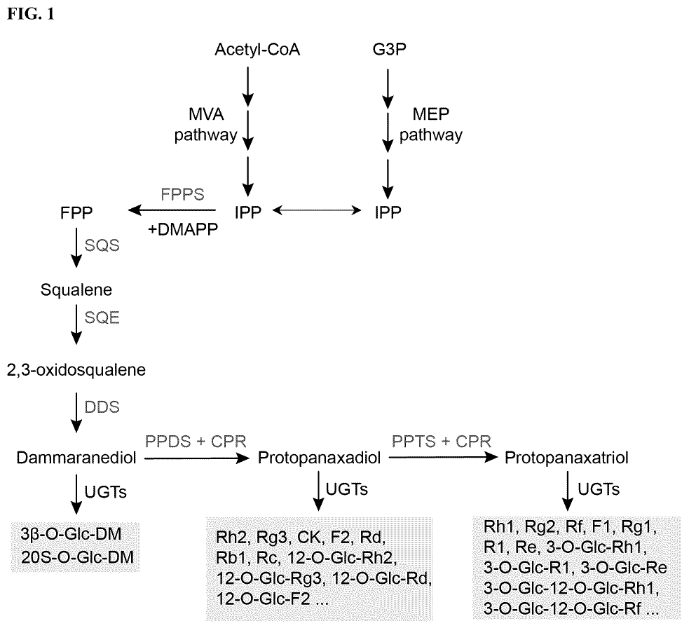

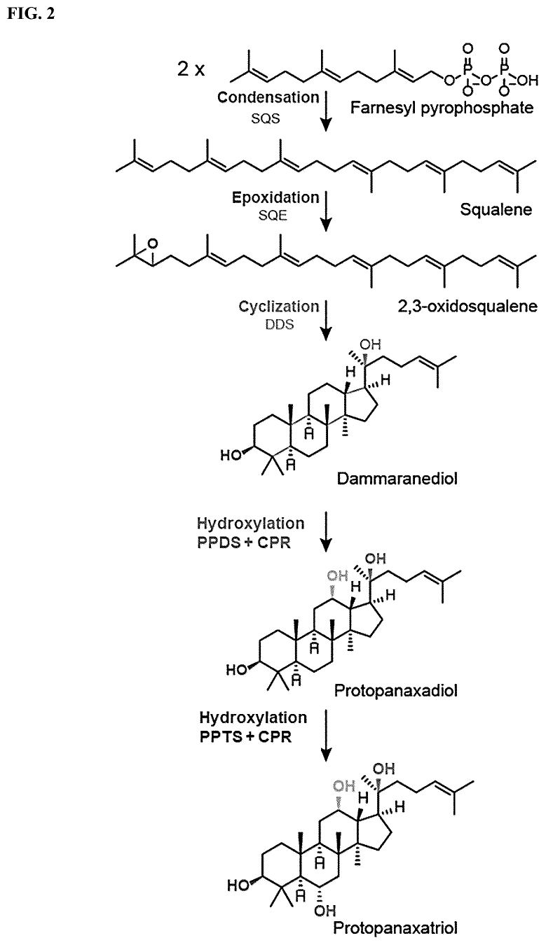

The biosynthetic pathways for dammarenediol, protopanaxadiol, and protopanaxatriol are illustrated in FIG. 2. As illustrated, two FPP molecules are converted to squalene via a condensation reaction, which is performed by a squalene synthase (SQS) enzyme. Epoxidation of squalene by squalene epoxidase (SQE) enzyme forms 2,3-oxidosqualene. Cyclization of 2,3-oxidosqualene by the DDS enzyme forms the dammarenediol core. Successive hydroxylations by the PPDS and PPTS enzymes form protopanaxadiol and protopanaxatriol, respectively. PPDS and PPTS are cytochrome P450 enzymes that are regenerated by reductase partners (CPR). UGT enzymes can also be employed to catalyze glycosylation(s) of C3-OH, C6-OH, and/or C20-OH. The biosynthesis pathway may be expressed in a microbial cell that produces iso-pentenyl pyrophosphate (IPP) and dimethylallyl pyrophosphate (DMAPP) such as a methylerythritol phosphate (MEP) pathway and/or a mevalonic acid (MVA) pathway. FIG. 1.

Accordingly, in one aspect, the present disclosure provides a method for producing dammarenediol or a derivative thereof. The method comprises providing a microbial host cell expressing a heterologous biosynthetic pathway producing dammarenediol or a derivative thereof. The heterologous biosynthetic pathway in various embodiments comprises one or more of: a dammarenediol synthase (DDS) enzyme comprising an amino acid sequence that has at least about 70% sequence identity to the amino acid sequence of SEQ ID NO: 3, SEQ ID NO: 4, or SEQ ID NO: 9; a protopanaxadiol synthase (PPDS) enzyme comprising an amino acid sequence that has at least 70% sequence identity to the amino acid sequence of SEQ ID NO: 10, SEQ ID NO: 11, SEQ ID NO: 12, SEQ ID NO: 13, SEQ ID NO: 14, SEQ ID NO: 15, or SEQ ID NO: 16; and a protopanaxatriol synthase (PPTS) enzyme comprising an amino acid sequence that has at least 70% sequence identity to the amino acid sequence of SEQ ID NO: 17, SEQ ID NO: 18, SEQ ID NO: 19, SEQ ID NO: 20, and SEQ ID NO: 21.

In another aspect, the present disclosure provides a microbial host cell producing dammarenediol or a derivative thereof. The microbial host cell expresses a heterologous biosynthetic pathway producing dammarenediol or a derivative thereof. In certain embodiments, the heterologous biosynthetic pathway comprises one or more of: a Dammarenediol Synthase (DDS) enzyme comprising an amino acid sequence that has at least about 70% sequence identity to the amino acid sequence of SEQ ID NO: 3, SEQ ID NO: 4, or SEQ ID NO: 9; a protopanaxadiol synthase (PPDS) enzyme comprising an amino acid sequence that has at least 70% sequence identity to the amino acid sequence of SEQ ID NO: 10, SEQ ID NO: 11, SEQ ID NO: 12, SEQ ID NO: 13, SEQ ID NO: 14, SEQ ID NO: 14, SEQ ID NO: 15, or SEQ ID NO: 16; and a protopanaxatriol synthase (PPTS) enzyme comprising an amino acid sequence that has at least 70% sequence identity to the amino acid sequence of SEQ ID NO: 17, SEQ ID NO: 18, SEQ ID NO: 19, SEQ ID NO: 20, and SEQ ID NO: 21.

In still other aspects, the invention provides engineered DDS, PPDS, and PPTS enzymes providing improved productivities or stabilities in microbial host cells, including for microbial production of dammarenediol, protopanaxadiol, and protopanaxatriol, and glycosylated forms thereof.

DDS is a component of the biosynthetic pathway for dammarane-type triterpene saponins (e.g. ginsenosides or panaxosides), which is an oxidosqualene cyclase that produces specifically the 20S isomer of the triterpene dammarenediol II shown in FIG. 2. Certain DDS enzymes disclosed herein are engineered to increase their stability, activity, expression and/or temperature resistance in microbial cells, such as bacterial cells.

In some embodiments, the DDS enzyme comprises an amino acid sequence having at least about 80% sequence identity, or at least about 85% sequence identity, or at least about 90% sequence identity, or at least about 95% sequence identity, or at least 97% sequence identity, or at least 98% sequence identity, or at least 99% sequence identity to the amino acid sequence of SEQ ID NO: 3, SEQ ID NO: 4, or SEQ ID NO: 9.

In some embodiments, the DDS enzyme comprises one or more mutations that are designed to improve the stability, activity, expression and/or temperature resistance of the DDS enzyme in a microbial strain. In some embodiments, the DDS enzyme comprises one or more mutations that are designed to improve stability, activity, expression and/or temperature resistance of the DDS enzyme in a bacterial host (e.g., E. coli). In some embodiments, the DDS enzyme comprises one or more mutations that are designed to improve stability, activity, expression and/or temperature resistance DDS enzyme in a yeast host.

In some embodiments, the DDS enzyme comprises an amino acid sequence having at least about 70% sequence identity, or at least about 80% sequence identity, or at least about 85% sequence identity, or at least about 90% sequence identity, or at least 95% sequence identity, or at least 97% sequence identity, or at least 98% sequence identity, or at least 99% sequence identity to SEQ ID NO: 3. In some embodiments, the DDS enzyme comprises from 1 to 30, or from 1 to 20, or from 1 to 10, or from 1 to 5 amino acid modifications independently selected from substitutions, deletions, and insertions with respect to SEQ ID NO: 3. In some embodiments, the DDS enzyme comprises one or more substitutions at positions corresponding to the following positions of SEQ ID NO: 3: 30, 64, 68, 365, 369, 425, 461, 465, 468, 606, 628, and 632. In some embodiments, the DDS enzyme comprises at least 2, or at least 3, or at least 4, or more substitutions at positions selected from 30, 64, 68, 365, 369, 425, 461, 465, 468, 606, 628, and 632 with respect to SEQ ID NO: 3.

In some embodiments, the DDS enzyme comprises one or more substitutions at positions corresponding to positions selected from 606, 628, and 632 of SEQ ID NO: 3. In some embodiments, the DDS enzyme comprises one or more substitutions selected from N606I, N606L, N606V, T628A, T628V, T628G, F632L, F632I, F632V, and F632A with respect to SEQ ID NO: 3. In some embodiments, the DDS enzyme comprises one or more substitutions selected from N606I, T628A, and F632L with respect to SEQ ID NO: 3. In some embodiments, the DDS enzyme comprises an amino acid sequence of SEQ ID NO: 5.

In some embodiments, the DDS enzyme comprising the substitutions selected from N606I, T628A, and F632L (with respect to SEQ ID NO: 3) comprises an amino acid sequence that otherwise has at least 95% sequence identity, or at least 97% sequence identity, or at least 98% sequence identity, or at least 99% sequence identity to SEQ ID NO: 5.

In some embodiments, the DDS enzyme comprises one or more substitutions at positions corresponding to positions selected from 365, 369, and 461 of SEQ ID NO: 3. In some embodiments, the DDS enzyme comprises one or more substitutions selected from T365E, T365D, F369Y, F369W, R461T, and R461S with respect to SEQ ID NO: 3. In some embodiments, the DDS enzyme comprises a substitution selected from T365E and T365D with respect to SEQ ID NO: 3. Additionally, or alternatively, the DDS enzyme comprises a substitution selected from F369Y and F369W with respect to SEQ ID NO: 3. Additionally, or alternatively, the DDS enzyme comprises a substitution selected from R461T and R461S with respect to SEQ ID NO: 3. In some embodiments, the DDS enzyme comprises one or more substitutions selected from T365E, F369Y, and R461S with respect to SEQ ID NO: 3. For example, the DDS enzyme may comprise the amino acid sequence of SEQ ID NO: 6.

In some embodiments, the DDS enzyme comprises one or more substitutions at positions selected from 30, 64, and 68 with respect to SEQ ID NO: 3. In some embodiments, the DDS enzyme comprises one or more substitutions selected from Q30D, Q30E, M64L, M64I, M64V, M64A, R68M, and R68T with respect to SEQ ID NO: 3. In some embodiments, the DDS enzyme comprises a substitutions selected from Q30D and Q30E with respect to SEQ ID NO: 3. Additionally or alternatively, the DDS enzyme comprises a substitution selected from M64L, M64I, M64V, and M64A with respect to SEQ ID NO: 3. Additionally or alternatively, the DDS enzyme comprises a substitution selected from R68M and R68T with respect to SEQ ID NO: 3. In some embodiments, the DDS enzyme comprises one or more substitutions selected from Q30D, M64L, and R68M with respect to SEQ ID NO: 3. In some embodiments, the DDS enzyme comprises the amino acid sequence of SEQ ID NO: 7.

In some embodiments, the DDS enzyme comprises one or more substitutions at positions selected from 425, 465, and 467 with respect to SEQ ID NO: 3. In some embodiments, the DDS enzyme comprises one or more substitutions selected from L465K, L465R, L465H, C468Y, C468F, C468W, I425G, I425V, and I425A with respect to SEQ ID NO: 3. In some embodiments, the DDS enzyme comprises a substitution selected from L465K, L465R, and L465H with respect to SEQ ID NO: 3. Additionally, or alternatively, the DDS enzyme comprises a substitution selected from C468Y, C468F, and C468W with respect to SEQ ID NO: 3. Additionally, or alternatively, the DDS enzyme comprises a substitution selected from I425G, I425V, and I425A with respect to SEQ ID NO: 3. In some embodiments, the DDS enzyme comprises one or more substitutions selected from L465K, C468Y, and I425A with respect to SEQ ID NO: 3. In some embodiments, the DDS enzyme comprises the amino acid sequence of SEQ ID NO: 8.

In some embodiments, the DDS enzyme comprises an amino acid sequence that has at least 90% sequence identity to SEQ ID NO: 7, and has one or more substitutions with respect to SEQ ID NO: 7 selected from: T364E, T364D, F368Y, R460S, R460T, L464K, L464R, C467Y, and I424A. For example, the DDS enzyme may have two, three, or more substitutions with respect to SEQ ID NO: 7 selected from: T364E, F368Y, R460S, L464K, C467Y, and I424A. In some embodiments, the DDS enzyme has at least the following substitutions with respect to SEQ ID NO: 7: T364E, F368Y, R460S, L464K, C467Y, and I424A. An exemplary engineered DDS enzyme has the amino acid sequence of SEQ ID NO: 81.

Accordingly, the present disclosure provides a DDS enzyme (including for use in the microbial host cells and methods of the disclosure), and which comprises an amino acid sequence that is at least 90% identical, or at least 95% identical, or at least 97% identical, or at least 98% identical to SEQ ID NO: 81. In various embodiments, the DDS enzyme comprises one or more mutations with respect to SEQ ID NO: 81 listed in Table 1. For example, the DDS enzyme may comprise two, three, four, five, or more mutations with respect to SEQ ID NO: 81 listed in Table 1. In exemplary embodiments, the DDS enzyme has one or more of the following mutations with respect to SEQ ID NO: 81: Y49F, S181T, deletion of amino acids L195-E197, S198P, E238S, I407V, D507E, R637K, and M695I. An exemplary engineered DDS enzyme comprises the amino acid sequence of SEQ ID NO: 82.

Accordingly, in some embodiments the DDS enzyme comprises an amino acid sequence that is at least 90% identical, or at least 95% identical, or at least 97% identical, or at least 98% identical to SEQ ID NO: 82. In some embodiments, the DDS enzyme comprises one or more mutations with respect to SEQ ID NO: 82 that are listed in Table 2. For example, the DDS enzyme may comprise two, three, four, five, or more mutations with respect to SEQ ID NO: 82 listed in Table 2. In various embodiments, the DDS enzyme comprises one or more mutations with respect to SEQ ID NO: 82 selected from: F649L, F649V, F649I, F649A, L548F, Q149E, Q149D, A120S, A120T, G573A, G573L, S380A, S380G, and A256G. Exemplary engineered DDS enzymes comprise two, three, four, five, or all mutations with respect to SEQ ID NO: 82 selected from: F649L, L548F, Q149E, A120S, G573A, S380A, and A256G.

An exemplary engineered DDS enzyme is represented by SEQ ID NO: 85. Thus, in some embodiments, the heterologous biosynthetic pathway comprises a DDS enzyme comprising an amino acid sequence that is at least 90%, or at least 95%, or at least 97%, or at least 98%, or at least 99% identical to SEQ ID NO: 85. For example, the DDS enzyme may have from 1 to 10 or from 1 to 5 amino acid modifications independently selected from substitutions, insertions, and deletions with respect to SEQ ID NO: 85.

Protopanaxadiol synthase (PPDS) is an oxidoreductase enzyme that converts dammarenediol to protopanaxadiol. Specifically, PPDS catalyzes the hydroxylation of dammarenediol at the C-12 position to yield protopanaxadiol as shown in FIG. 2. Certain PPDS enzymes disclosed herein are engineered to increase their stability, activity, expression and/or temperature resistance in the microbial cells.

In some embodiments, the heterologous biosynthetic pathway comprises a PPDS enzyme comprising an amino acid sequence that has at least about 70% sequence identity to the amino acid sequence of SEQ ID NO: 10, SEQ ID NO: 11, SEQ ID NO: 12, SEQ ID NO: 13, SEQ ID NO: 14, SEQ ID NO: 15, or SEQ ID NO: 16. In some embodiments, the PPDS enzyme comprises an amino acid sequence having at least about 70%, or at least about 80%, or at least about 85%, or at least about 90%, or at least 95%, or at least 98%, or at least 99% sequence identity to an amino acid sequence selected from SEQ ID NOS: 10, 11, 12 and 16. In some embodiments, the PPDS enzyme comprises an amino acid sequence having at least about 70%, or at least about 80%, or at least about 85%, or at least about 90%, or at least 95%, or at least 98%, or at least 99% sequence identity to the amino acid sequence of SEQ ID NO: 10. In some embodiments, the PPDS enzyme comprises from 1 to 30, or from 1 to 20, or from 1 to 10, or from 1 to 5 amino acid modifications independently selected from substitutions, deletions, and insertions with respect to SEQ ID NO: 10.

In various embodiments, the PPDS enzyme comprises one or more substitutions (e.g., at least 2, 3, 4, or 5 substitutions) selected from N58D, N58E, S68P, R85K, I95V, L96F, L96W, L96Y, T108N, T108Q, D135E, M144L, M144V, M144I, V150P, G152A, G152L, G152I, G152V, M153L, M153I, M153V, M153A, F167H, S192A, S192G, E202P, I212F, R243K, R243H, V248I, V248L,N277D, N277E, Q278E, Q278D, L283M, L292I, L292V, L292A, F317L, F317I, F317V, F317A, V329M, N333K, N333R, K338G, K338A, L346I, R347Q, R347N, I362L, I362V, I362A, M390L, M390I, M390V, M390A, H482R, and H482K with respect to SEQ ID NO: 10. In various embodiments, the PPDS enzyme comprises at least 2, or at least 3, or at least 4, or at least 5, or at least 8, or at least 10 amino acid substitutions shown in Table 3.

In exemplary embodiments, the PPDS enzyme comprises at least one, two, three, four, five, six, seven, eight, or all of the following mutations with respect to SEQ ID NO: 10: T108N, I212F, K338G, D135E, S68P, V150P, F167H, L283M, H482R, R347Q, M390L, R243K, L292I, V329M, Q278E, and N58E.

An exemplary engineered PPDS enzyme is represented by SEQ ID NO: 83. Thus, in some aspects and embodiments the heterologous biosynthetic pathway comprises a PPDS enzyme that comprises the amino acid sequence of SEQ ID NO: 83, or comprises a PPTS enzyme comprising an amino acid sequence that is at least 90%, or at least 95%, or at least 97%, or at least 98%, or at least 99% identical to SEQ ID NO: 83. In some embodiments, the PPDS enzyme comprises an amino acid sequence that has from 1 to 10 or from 1 to 5 amino acid modifications from SEQ ID NO: 83, the modification being independently selected from substitutions, insertions, and deletions.

Protopanaxatriol synthase (PPTS) catalyzes the formation of protopanaxatriol from protopanaxadiol. PPTS is an oxidoreductase enzyme that catalyzes the hydroxylation of protopanaxadiol at the C-6 position to yield protopanaxatriol as shown in FIG. 2. Certain PPTS enzymes disclosed herein are engineered to increase their stability, activity, expression and/or temperature resistance in the microbial cells.

In some embodiments, the heterologous biosynthetic pathway comprises a PPTS enzyme comprising an amino acid sequence that has at least about 70% sequence identity to an amino acid sequence selected from SEQ ID NO: 17, SEQ ID NO: 18, SEQ ID NO: 19, SEQ ID NO: 20 and SEQ ID NO: 21. In some embodiments, the heterologous biosynthetic pathway comprises a PPTS enzyme comprising an amino acid sequence that has at least about 70% sequence identity, or at least about 80% sequence identity, or at least about 85% sequence identity, or at least about 90% sequence identity, or at least 95% sequence identity, or at least 97% sequence identity, or at least 98% sequence identity to an amino acid sequence selected from SEQ ID NO: 17, SEQ ID NO: 18, SEQ ID NO: 19, SEQ ID NO: 20 and SEQ ID NO: 21. In some embodiments, the PPTS enzyme comprises an amino acid sequence having at least about 70%, or at least about 80%, or at least about 85%, or at least about 90%, or at least 95%, or at least 98%, or at least 99% sequence identity to the amino acid sequence of SEQ ID NO: 17. In some embodiments, the PPTS enzyme comprises from 1 to 30, or 1 to 20, or from 1 to 10, or from 1 to 5 amino acid modifications independently selected from substitutions, deletions, and insertions with respect to SEQ ID NO: 17.

In some embodiments, the PPTS enzyme comprises one or more substitutions (e.g., at least 2, 3, 4, or 5 substitutions) with respect to SEQ ID NO: 17 selected from: I98L, I98V, I98A, A113S, A120S, A120T, K146R, F147Y, S166K, S166R, E176K, E176R, W185R, W185K, L187F, L187Y, L187W, L215I, F217L, F217I, F217V, F217A, V239I, V239L, V239A, R244K, K247L, K247V, K247I, K247A, Q249E, Q249D, T250P, K252Q, K252N, M259L, M259I, M259V, V278I, G294T, G294S, A323P, E324G, E324A, S328N, S328Q, R334K, V358E, V358D, V359A, V359G, K362D, K362E, S364T, N367G, N367A, I369T, I369S, K391P, M407A, M407G, F409Y, I412V, F426Y, V431I, V431L, N463K, N463R, and C472H.

An exemplary engineered PPTS enzyme is represented by SEQ ID NO: 84. Thus, in some aspects and embodiments the heterologous biosynthetic pathway comprises a PPTS enzyme that comprises the amino acid sequence of SEQ ID NO: 84, or comprises a PPTS enzyme comprising an amino acid sequence that is at least 90%, or at least 95%, or at least 97%, or at least 98%, or at least 99% identical to SEQ ID NO: 84. In some embodiments, the PPDS enzyme comprises an amino acid sequence that has from 1 to 10 or from 1 to 5 amino acid modifications from SEQ ID NO: 84, the modification being independently selected from substitutions, insertions, and deletions.

In some embodiments, the PPTS enzyme comprises at least 2, or at least 3, or at least 4, or at least 5, or at least 8, or at least 10 amino acid substitutions with respect to SEQ ID NO: 17 listed in Table 4. Exemplary PPTS enzymes comprise at least 2, at least 3, at least 4, at least 5, or all amino acid substitutions (with respect to SEQ ID NO: 17) selected from: G294T, S166K, C472H, K252Q, V239I, A323P, I412V, I369T, K362D, and T250P.

In some embodiments, the heterologous enzyme pathway further comprises one or more uridine diphosphate-dependent glycosyltransferase (UGT) enzymes, thereby producing one or more glycosides of dammarenediol, protopanaxadiol or protopanaxatriol that are shown in FIG. 1. The dammarenediol glycosides, protopanaxadiol glycosides or protopanaxatriol glycosides may have from 1 to about 6 glycosyl groups. In other embodiments, one or more glycosylations occur in vitro (e.g., using a cell free reaction), or is conducted using a bioconversion reaction in which the dammarenediol, protopanaxadiol or protopanaxatriol substrate is fed to a cell culture that expresses the one or more UGT enzymes.

In some embodiments, dammarenediol is monoglycosylated. In some embodiments, the dammarenediol is diglycosylated. In some embodiments, dammarenediol is glycosylated at C3-OH and/or C20-OH. In some embodiments, the dammarenediol glycosides comprise 33-O glucosylation and/or 20S—O glucosylation. In some embodiments, the dammarenediol glycosides comprise one or more branching glycosylations.

In some embodiments, protopanaxadiol is monoglycosylated. In some embodiments, the protopanaxadiol is diglycosylated. In some embodiments, protopanaxadiol is glycosylated at C3-OH and/or C20-OH. In some embodiments, the protopanaxadiol glycosides comprise one or more branching glycosylations.

In some embodiments, protopanaxatriol is monoglycosylated. In some embodiments, the protopanaxatriol is diglycosylated. In some embodiments, the protopanaxatriol is triglycosylated. In some embodiments, protopanaxatriol is glycosylated at C3-OH, C6-OH, and/or C20-OH. In some embodiments, the protopanaxatriol glycosides comprise one or more branching glycosylations.

In some embodiments, the microbial host cell is capable of producing dammarenediol, protopanaxadiol or protopanaxatriol as a substrate for glycosylation by one or more UGT enzymes. In some embodiments, the UGT enzyme(s) are capable of catalyzing glycosylation of C3-OH and/or C20-OH of dammarenediol. In some embodiments, the UGT enzyme(s) are capable of catalyzing glycosylation of C3-OH and/or C20-OH of protopanaxadiol. In some embodiments, the UGT enzyme(s) are capable of catalyzing glycosylation of C3-OH, C6-OH, and/or C20-OH of protopanaxatriol. For example, in some embodiments, the microbial cell expresses at least one, or at least two, or at least three UGT enzymes, resulting in glucosylation of dammarenediol, protopanaxadiol or protopanaxatriol. Exemplary UGT enzymes that can glycosylate a triterpenoid core include those described in WO 2021/126960, which is hereby incorporated by reference in its entirety. In some embodiments, the UGT enzyme(s) further catalyze one or more branching glycosylations, such 1-2, 1-3, and 1-6 branching glycosylations. In various embodiments, the glycosylation reaction transfers monosaccharide units selected from glucosyl, arabinosyl, furanosyl, rhamnosyl, and xylosyl.

In some embodiments, the heterologous biosynthetic pathway further comprises a squalene synthase (SQS) enzyme, catalyzing synthesis of squalene from farnesyl diphosphate. In some embodiments, the SQS comprises an amino acid sequence having at least 70%, or at least 80%, or at least 85%, or at least 90%, or at least 95%, or at least 98%, or at least 99% sequence identity to an amino acid sequence selected from SEQ ID NOs: 1 and 23-38.

In some embodiments, the SQS comprises an amino acid sequence that is at least 70% identical to Artemisia annua SQS (SEQ ID NO: 1). For example, the SQS may comprise an amino acid sequence that is at least 80%, or at least 85%, or at least 90%, or at least 95%, or at least 98%, or at least 99% identical to SEQ ID NO: 1. In some embodiments, the SQS comprises an amino acid sequence having from 1 to 20 amino acid modifications or from 1 to 10 amino acid modifications with respect to SEQ ID NO: 1, the amino acid modifications being independently selected from amino acid substitutions, deletions, and insertions. Amino acid modifications may be made to increase expression or stability of the enzyme in the microbial cell, or to increase productivity of the enzyme. AaSQS has high activity in E. coli.

In some embodiments, the SQS comprises an amino acid sequence that is at least 70% identical to Siraitia grosvenorii SQSa (SEQ ID NO: 23). For example, the SQS may comprise an amino acid sequence that is at least 80%, or at least 85%, or at least 90%, or at least 95%, or at least 98%, or at least 99% identical to SEQ ID NO: 23. In some embodiments, the SQS comprises an amino acid sequence having from 1 to 20 amino acid modifications or from 1 to 10 amino acid modifications with respect to SEQ ID NO: 23, the amino acid modifications being independently selected from amino acid substitutions, deletions, and insertions. Amino acid modifications may be made to increase expression or stability of the enzyme in the microbial cell, or to increase productivity of the enzyme. SgSQSa has high activity in E. coli.

In some embodiments, the SQS comprises an amino acid sequence that is at least 70% identical to Siraitia grosvenorii SQSb (SEQ ID NO: 24). For example, the SQS may comprise an amino acid sequence that is at least 80%, or at least 85%, or at least 90%, or at least 95%, or at least 98%, or at least 99% identical to SEQ ID NO: 24. In some embodiments, the SQS comprises an amino acid sequence having from 1 to 20 amino acid modifications or from 1 to 10 amino acid modifications with respect to SEQ ID NO: 24, the amino acid modifications being independently selected from amino acid substitutions, deletions, and insertions. Amino acid modifications may be made to increase expression or stability of the enzyme in the microbial cell, or to increase productivity of the enzyme. SgSQSb has high activity in E. coli.

Amino acid modifications to the SQS enzyme can be guided by available enzyme structures and homology models, including those described in Aminfar and Tohidfar, In silico analysis of squalene synthase in Fabaceae family using bioinformatics tools, J. Genetic Engineer. and Biotech. 16 (2018) 739-747. The publicly available crystal structure for HsSQE (PDB entry: 6C6N) may be used to inform amino acid modifications.

In some embodiments, the heterologous biosynthetic pathway further comprises a squalene epoxidase (SQE) producing 2,3-oxidosqualene. In some embodiments, the SQE comprises an amino acid sequence having at least 70%, or at least 80%, or at least 85%, or at least 90%, or at least 95%, or at least 98%, or at least 99% sequence identity to an amino acid sequence selected from SEQ ID NOs: 2 and 39-70. In some embodiments, the SQE enzyme comprises an amino acid sequence having at least 70%, or at least 80%, or at least 85%, or at least 90%, or at least 95%, or at least 98%, or at least 99% sequence identity to the amino acid sequence of SEQ ID NO: 2.

Amino acid modifications can be guided by available enzyme structures and homology models, including those described in Padyana A K, et al., Structure and inhibition mechanism of the catalytic domain of human squalene epoxidase, Nat. Comm. (2019) Vol. 10(97): 1-10; or Ruckenstulh et al., Structure-Function Correlations of Two Highly Conserved Motifs in Saccharomyces cerevisiae Squalene Epoxidase, Antimicrob. Agents and Chemo. (2008) Vol. 52(4): 1496-1499.

In some embodiments, the microbial host cell expresses an enzymatic pathway that produces iso-pentenyl diphosphate (IPP) and dimethylallyl diphosphate (DMAPP). In some embodiments, the enzymatic pathway is a methylerythritol phosphate (MEP) pathway and/or a mevalonic acid (MVA) pathway.

In some embodiments, the host cell is a bacterial host cell engineered to increase production of IPP and DMAPP from glucose as described in U.S. Pat. Nos. 10,480,015 and 10,662,442, the contents of which are hereby incorporated by reference in their entireties. For example, in some embodiments the host cell overexpresses MEP pathway enzymes, with balanced expression to push/pull carbon flux to IPP and DMAPP. In some embodiments, the host cell is engineered to increase the availability or activity of Fe—S cluster proteins, so as to support higher activity of IspG and IspH, which are Fe—S enzymes. In some embodiments, the host cell is engineered to overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux and/or terpenoid production. In some embodiments, the host cell exhibits higher activity of IspH relative to IspG. In some embodiments, the host cell is engineered to downregulate the ubiquinone biosynthesis pathway, e.g., by reducing the expression or activity of IspB, which uses IPP and FPP substrate.

The microbial cell will produce MEP or MVA products, which act as substrates for the heterologous enzyme pathway. The MEP (2-C-methyl-D-erythritol 4-phosphate) pathway, also called the MEP/DOXP (2-C-methyl-D-erythritol 4-phosphate/1-deoxy-D-xylulose 5-phosphate) pathway or the non-mevalonate pathway or the mevalonic acid-independent pathway refers to the pathway that converts glyceraldehyde-3-phosphate and pyruvate to IPP and DMAPP. The pathway, which is present in bacteria, typically involves action of the following enzymes: 1-deoxy-D-xylulose-5-phosphate synthase (Dxs), 1-deoxy-D-xylulose-5-phosphate reductoisomerase (IspC), 4-diphosphocytidyl-2-C-methyl-D-erythritol synthase (IspD), 4-diphosphocytidyl-2-C-methyl-D-erythritol kinase (IspE), 2C-methyl-D-erythritol 2,4-cyclodiphosphate synthase (IspF), 1-hydroxy-2-methyl-2-(E)-butenyl 4-diphosphate synthase (IspG), and isopentenyl diphosphate isomerase (IspH). The MEP pathway, and the genes and enzymes that make up the MEP pathway, are described in U.S. Pat. No. 8,512,988, which is hereby incorporated by reference in its entirety. For example, genes that make up the MEP pathway include dxs, ispC, ispD, ispE, ispF, ispG, ispH, idi, and ispA. In some embodiments, the host cell expresses or overexpresses one or more of dxs, ispC, ispD, ispE, ispF, ispG, ispH, idi, ispA, or modified variants thereof, which results in the increased production of IPP and DMAPP. In some embodiments, the FPP substrate is produced at least in part by metabolic flux through an MEP pathway, and wherein the host cell has at least one additional gene copy of one or more of dxs, ispC, ispD, ispE, ispF, ispG, ispH, idi, ispA, or modified variants thereof.

The MVA pathway refers to the biosynthetic pathway that converts acetyl-CoA to IPP. The mevalonate pathway, which will be present in yeast, typically comprises enzymes that catalyze the following steps: (a) condensing two molecules of acetyl-CoA to acetoacetyl-CoA (e.g., by action of acetoacetyl-CoA thiolase); (b) condensing acetoacetyl-CoA with acetyl-CoA to form hydroxymethylglutaryl-CoenzymeA (HMG-CoA) (e.g., by action of HMG-CoA synthase (HMGS)); (c) converting HMG-CoA to mevalonate (e.g., by action of HMG-CoA reductase (HMGR)); (d) phosphorylating mevalonate to mevalonate 5-phosphate (e.g., by action of mevalonate kinase (MK)); (e) converting mevalonate 5-phosphate to mevalonate 5-pyrophosphate (e.g., by action of phosphomevalonate kinase (PMK)); and (f) converting mevalonate 5-pyrophosphate to isopentenyl pyrophosphate (e.g., by action of mevalonate pyrophosphate decarboxylase (MPD)). The MVA pathway, and the genes and enzymes that make up the MVA pathway, are described in U.S. Pat. No. 7,667,017, which is hereby incorporated by reference in its entirety. In some embodiments, the host cell expresses or overexpresses one or more of acetoacetyl-CoA thiolase, HMGS, HMGR, MK, PMK, and MPD or modified variants thereof, which results in the increased production of IPP and DMAPP. In some embodiments, FPP substrate is produced at least in part by metabolic flux through an MVA pathway, and wherein the host cell has at least one additional gene copy of one or more of acetoacetyl-CoA thiolase, HMGS, HMGR, MK, PMK, MPD, or modified variants thereof.

In various embodiments, the microbial cells further express one or more farnesyl diphosphate synthase (FPPS) enzymes. An exemplary enzyme is shown herein as SEQ ID NO: 80. Numerous other FPPS enzymes are well known in the art and the selection of which is not critical.

In still other embodiments, microbial cells expressing the heterologous biosynthesis pathway co-express an isoprenol utilization pathway as described in US 2019/0367950, which is hereby incorporated by reference in its entirety. Such cells can produce IPP and DMAPP precursors from prenol and/or isoprenol substrate provided to the culture.

The microbial host cell in various embodiments may be prokaryotic or eukaryotic. In some embodiments, the microbial host cell is a bacterium, and which can be optionally selected from Escherichia spp., Bacillus spp., Corynebacterium spp., Rhodobacter spp., Zymomonas spp., Vibrio spp., and Pseudomonas spp. For example, in some embodiments, the bacterial host cell is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterial host cell is E. coli. Alternatively, the microbial cell may be a yeast cell, such as but not limited to a species of Saccharomyces, Pichia, or Yarrowia, including Saccharomyces cerevisiae, Pichia pastoris, and Yarrowia lipolytica.

In some embodiments, the microbial host cell is cultured in a carbon source comprising glucose, sucrose, fructose, xylose, and/or glycerol. In some embodiments, culture conditions are selected from aerobic, microaerobic, and anaerobic. In some embodiments, the microbial host cell is cultured at a temperature in the range of about 22° C. to about 37° C., or about 27° C. to about 37° C., or about 30° C. to about 37° C. In some embodiments, dammarenediol, protopanaxadiol, protopanaxatriol or glycosylated derivatives thereof are recovered from the culture.

In various embodiments, the microbial host cell may be cultured at a temperature between 22° C. and 37° C. While commercial biosynthesis in bacteria such as E. coli can be limited by the temperature at which overexpressed and/or foreign enzymes (e.g., enzymes derived from plants) are stable, recombinant enzymes may be engineered to allow for cultures to be maintained at higher temperatures, resulting in higher yields and higher overall productivity. In some embodiments, the culturing is conducted at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C.

In some embodiments, the microbial host cells are further suitable for commercial production, at commercial scale. In some embodiments, the size of the culture is at least about 100 L, at least about 200 L, at least about 500 L, at least about 1,000 L, or at least about 10,000 L, or at least about 100,000 L, or at least about 500,000 L, or at least about 600,000 L. In an embodiment, the culturing may be conducted in batch culture, continuous culture, or semi-continuous culture.

In various embodiments, methods further include recovering the product from the cell culture or from cell lysates. In some embodiments, the culture produces at least about 100 mg/L, or at least about 200 mg/L, or at least about 500 mg/L, or at least about 1 g/L, or at least about 2 g/L, or at least about 5 g/L, or at least about 10 g/L, or at least about 20 g/L, or at least about 30 g/L, or at least about 40 g/L of the terpenoid or terpenoid glycoside product.

In some embodiments, the production of indole (including prenylated indole) is used as a surrogate marker for terpenoid production, and/or the accumulation of indole in the culture is controlled to increase production. For example, in various embodiments, accumulation of indole in the culture is controlled to below about 100 mg/L, or below about 75 mg/L, or below about 50 mg/L, or below about 25 mg/L, or below about 10 mg/L. The accumulation of indole can be controlled by balancing protein expression and activity using the multivariate modular approach as described in U.S. Pat. No. 8,927,241 (which is hereby incorporated by reference), and/or is controlled by chemical means.

Manipulation of the expression of genes and/or proteins, including gene modules, can be achieved through various methods. For example, expression of the genes or operons can be regulated through selection of promoters, such as inducible or constitutive promoters, with different strengths (e.g., strong, intermediate, or weak). Several non-limiting examples of promoters of different strengths include Trc, T5 and T7. Additionally, expression of genes or operons can be regulated through manipulation of the copy number of the gene or operon in the cell. In some embodiments, expression of genes or operons can be regulated through manipulating the order of the genes within a module, where the genes transcribed first are generally expressed at a higher level. In some embodiments, expression of genes or operons is regulated through integration of one or more genes or operons into the chromosome.

Optimization of protein expression can also be achieved through selection of appropriate promoters and ribosomal binding sites. In some embodiments, this may include the selection of high-copy number plasmids, or single-, low- or medium-copy number plasmids. The step of transcription termination can also be targeted for regulation of gene expression, through the introduction or elimination of structures such as stem-loops.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA. The heterologous DNA is placed under operable control of transcriptional elements to permit the expression of the heterologous DNA in the host cell.

In some embodiments, endogenous genes are edited, as opposed to gene complementation. Editing can modify endogenous promoters, ribosomal binding sequences, or other expression control sequences, and/or in some embodiments modifies trans-acting and/or cis-acting factors in gene regulation. Genome editing can take place using CRISPR/Cas genome editing techniques, or similar techniques employing zinc finger nucleases and TALENs. In some embodiments, the endogenous genes are replaced by homologous recombination.

In some embodiments, genes are overexpressed at least in part by controlling gene copy number. While gene copy number can be conveniently controlled using plasmids with varying copy number, gene duplication and chromosomal integration can also be employed. For example, a process for genetically stable tandem gene duplication is described in US 2011/0236927, which is hereby incorporated by reference in its entirety.

The terpene or terpenoid product can be recovered by any suitable process. For example, the aqueous phase can be recovered, and/or the whole cell biomass can be recovered, for further processing. The desired product can be produced in batch or continuous bioreactor systems.

For example, products may be recovered from the reaction or culture, which can include adjusting the pH and/or temperature of the reaction or culture, and optionally adding one or more solubilizers, followed by enzyme or biomass removal. Biomass and/or enzymes can be removed by centrifugation, thereby preparing a clarified broth. An exemplary process for biomass removal employs a disc stack centrifuge to separate liquid and solid phases. The clarified broth (liquid phase) is recovered for further processing. In some embodiments, products are crystallized from the clarified broth, and/or may be purified from the clarified broth using one or more processes selected from filtration, ion exchange, activated charcoal, bentonite, affinity chromatography, and digestion, which can optionally be conducted prior to crystallization and/or prior to recrystallization. In some embodiments, the recovery process can include one or more steps of tangential flow filtration (TFF). Exemplary processes for recovery of glycosylated products are described in WO 2022/115527, which is hereby incorporated by reference in its entirety. Other process for product recovery, including for recovery of triterpenoids (such as squalene derivatives) is described in US 2021/0207078, which is hereby incorporated by reference in its entirety.

The similarity of nucleotide and amino acid sequences, i.e. the percentage of sequence identity, can be determined via sequence alignments. Such alignments can be carried out with several art-known algorithms, such as with the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80). The grade of sequence identity (sequence matching) may be calculated using e.g. BLAST, BLAT or BlastZ (or BlastX). A similar algorithm is incorporated into the BLASTN and BLASTP programs of Altschul et al (1990) J. Mol. Biol. 215: 403-410. BLAST polynucleotide searches can be performed with the BLASTN program, score=100, word length=12.

BLAST protein searches may be performed with the BLASTP program, score=50, word length=3. To obtain gapped alignments for comparative purposes, Gapped BLAST is utilized as described in Altschul et al (1997) Nucleic Acids Res. 25: 3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups:
(1) hydrophobic: Met, Ala, Val, Leu, Ile;
(2) neutral hydrophilic: Cys, Ser, Thr; Asn, Gln;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe.

As used herein, “non-conservative substitutions” are defined as exchanges of an amino acid by another amino acid listed in a different group of the six standard amino acid groups (1) to (6) shown above.

Modifications of enzymes as described herein can include conservative and/or non-conservative mutations. In some embodiments, an Alanine is substituted or inserted at position 2, to increase stability.

In some embodiments “rational design” is involved in constructing specific mutations in enzymes. Rational design refers to incorporating knowledge of the enzyme, or related enzymes, such as its reaction thermodynamics and kinetics, its three dimensional structure, its active site(s), its substrate(s) and/or the interaction between the enzyme and substrate, into the design of the specific mutation. Based on a rational design approach, mutations can be created in an enzyme which can then be screened for increased production of a terpene or terpenoid relative to control levels. In some embodiments, mutations can be rationally designed based on homology modeling. As used herein, “homology modeling” refers to the process of constructing an atomic resolution model of one protein from its amino acid sequence and a three-dimensional structure of a related homologous protein.

The dammarenediol, protopanaxadiol, protopanaxatriol, or ginsenoside derived therefrom obtained according to this disclosure can be incorporated into pesticide or insecticide compositions. In some embodiments, the product is incorporated into a pharmaceutical composition for use as an active pharmaceutical agent having anti-inflammatory, anxiolytic, anti-stress, and anti-tumor activity. In some embodiments, the product is incorporate into food products (including beverages) and nutraceutical products.

As used in this specification and the appended claims, the singular forms “a”, “an” and “the” include plural referents unless the content clearly dictates otherwise. For example, reference to “a cell” includes a combination of two or more cells, and the like.

As used herein, the term “about” in reference to a number is generally taken to include numbers that fall within a range of 10% in either direction (greater than or less than) of the number.

### Examples

### Example 1. The Biosynthetic Pathway of Protopanaxatriol

Protopanaxatriol can be produced by biosynthetic fermentation processes using microbial strains that produce high levels of MVA or MEP pathway products, along with heterologous expression of the biosynthesis enzymes. For example, in bacteria such as E. coli, isopentenyl pyrophosphate (IPP) and dimethylallyl pyrophosphate (DMAPP) can be produced from glucose or other carbon substrates, and converted to farnesyl diphosphate (FPP) by recombinant farnesyl diphosphate synthase (FPPS). FIG. 2 illustrates a biosynthetic pathway for production of protopanaxatriol in microbial cells from FPP. Two FPP molecules are converted to squalene via a condensation reaction, which is performed by a squalene synthase (SQS). Epoxidation of squalene by squalene epoxidase (SQE) forms 2,3-oxidosqualene. Cyclization of 2,3-oxidosqualene by dammarenediol synthase (DDS) forms the dammarenediol-II core. Successive hydroxylations form protopanaxadiol (via protopanaxadiol synthase, PPDS) and protopanaxatriol (via protopanaxatriol synthase, PPTS). PPDS and PPTS are cytochrome P450 enzymes that are regenerated by reductase partners (CPR).

An E. coli strain that has high MEP pathway flux may be used (see U.S. Pat. Nos. 10,662,442 and 10,480,015, which are hereby incorporated by reference), to direct the MEP pathway products to protopanaxatriol.

### Example 2. Production of Squalene, Oxidosqualene and Dammarenediol

In these experiments, an SQS enzyme and a SQE enzyme were co-expressed with a DDS enzyme in an E coli strain producing farnesyl pyrophosphate (FPP), and the production of squalene, 2,3-oxidosqualene and dammarenediol was quantified by GC-FID chromatography using authentic standards of each compound. Dammarenediol productions were verified by GC-MS spectrum analysis. The SQS enzyme (designated SQS1) is shown herein as SEQ ID NO: 1. The SQE enzyme (designated SQE1) is shown herein as SEQ ID NO: 2. The FPPS enzyme is shown herein as SEQ ID NO: 80. Candidate DDS enzymes include those designated as DDS1 (SEQ ID NO: 3) and DDS7 (SEQ ID NO: 9).

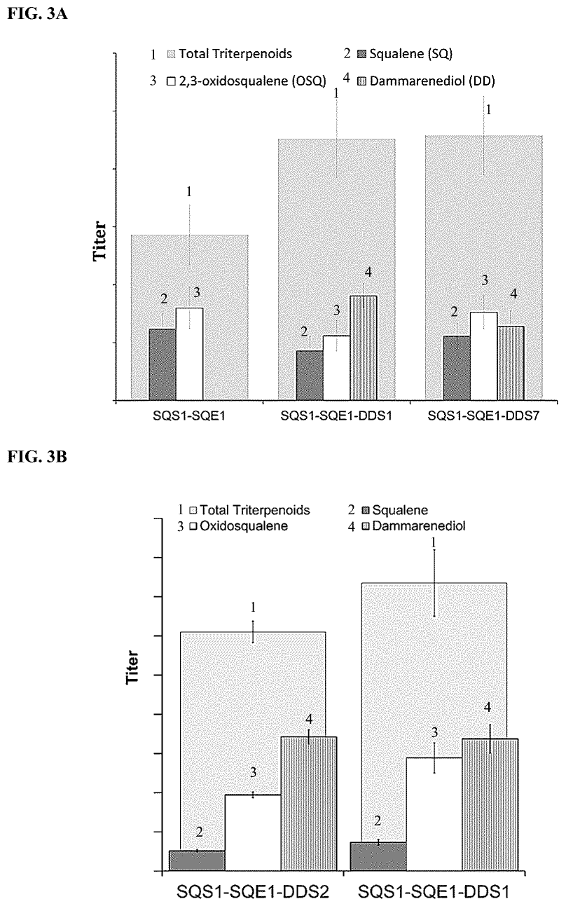

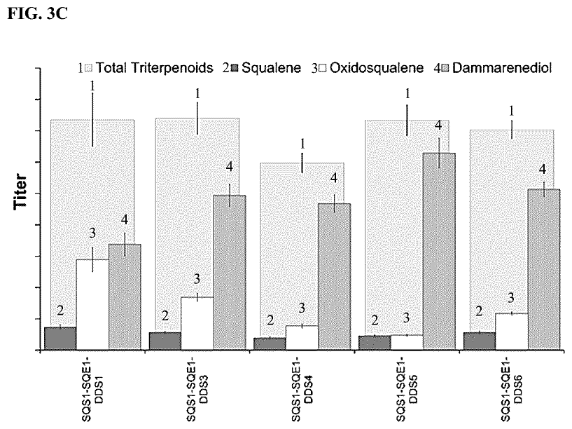

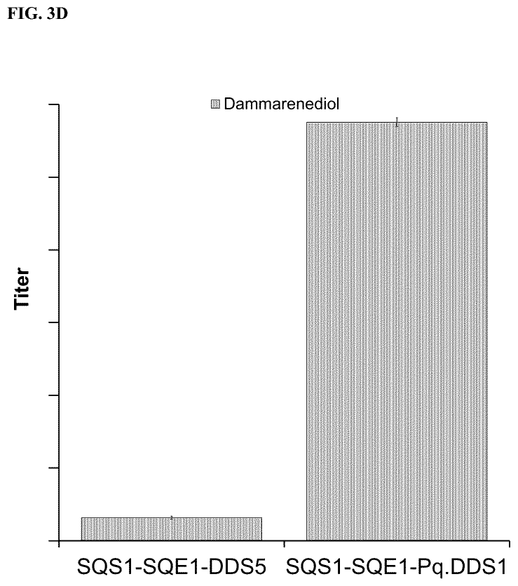

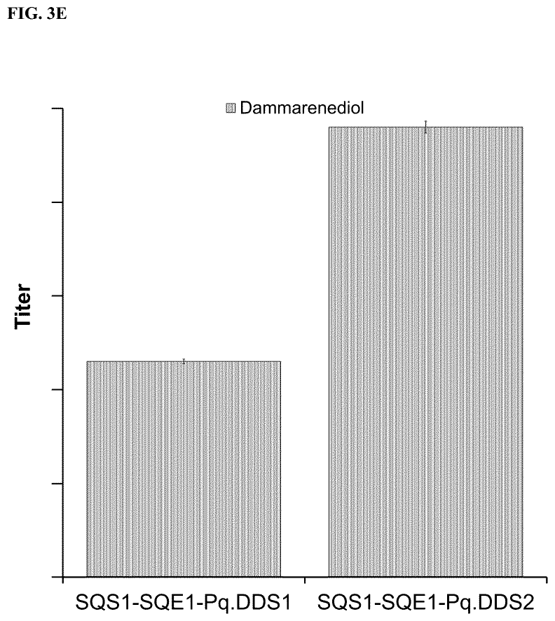

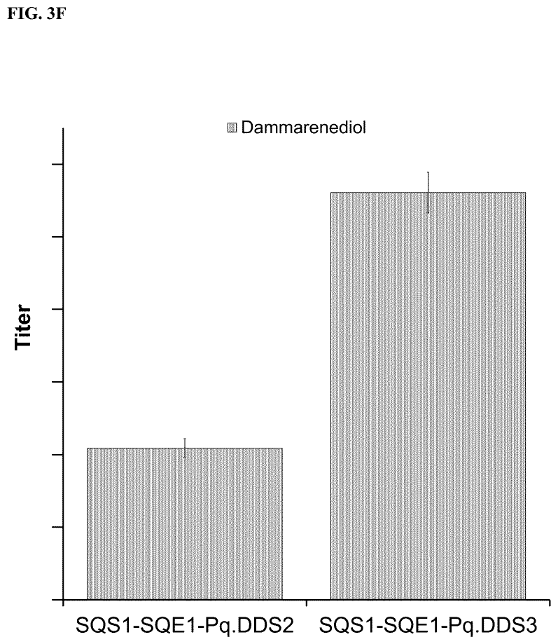

E. coli strains were incubated at 30° C. for 72 hr. The titers of squalene, oxidosqualene, dammarenediol and total triterpenoids were plotted. Dammarenediol-II productions were verified by GC-MS spectrum analysis. As shown in FIG. 3A, only the strains expressing DDS1 or DDS7 produced dammarenediol (center and right).

Two DDS enzymes, DDS2 (SEQ ID NO: 4) and DDS1 (SEQ ID NO: 3), were expressed in the E. coli strain and levels of the dammarenediol and intermediates were compared. These strains were incubated at 30° C. for 72 hr, and squalene, oxidosqualene and dammarenediol were quantified. The titers of squalene, oxidosqualene, dammarenediol and total triterpenoids were plotted. As shown in FIG. 3B, the strains expressing DDS2 or DDS1 produced dammarenediol. Dammarenediol productions were verified by GC-MS spectrum analysis.

These results demonstrate that a bacterial strain co-expressing FPPS, a squalene synthase (SQS), a squalene epoxidase (SQE) and a dammarenediol synthase (DDS) can produce dammarenediol.

To improve the production of dammarenediol, DDS1 enzyme was engineered for improved production of dammarenediol. Specifically, the following DDS derivatives were constructed:
(1) a derivative of DDS1 harboring the following substitutions: N606I, T628A, and F632L (DDS3, SEQ ID NO: 5);
(2) a derivative of DDS1 harboring the following substitutions: T365E, F369Y, and R461S (DDS4, SEQ ID NO: 6);
(3) a derivative of DDS1 harboring the following substitutions: Q30D, M64L, and R68M (DDS5, SEQ ID NO: 7);
(4) a derivative of DDS1 harboring the following substitutions: L465K, C468Y, and I425A (DDS6, SEQ ID NO: 8).

These derivatives were expressed in MEP-pathway engineered E. coli expressing FPPS, SQS, and SQE enzymes. These strains were incubated at 30° C. for 72 h, and squalene, oxidosqualene and dammarenediol were quantified by GC-FID chromatography using authentic standards. As shown in FIG. 3C, strains expressing each of DDS3 to DDS6 produced higher titers of dammarenediol as compared to a strain expressing DDS1 (FIG. 3C). Dammarenediol productions were verified by GC-MS spectrum analysis. Engineered DDS derivatives (DDS3-6) in FIG. 3C show improvements of enzyme stability in the strains. In particular, DDS5 exhibits a substantial increase of dammarenediol titer relative to DDS1.

The DDS1 derivative (Pq.DDS1) (SEQ ID NO: 80) was tested alongside DDS5. Pq.DDS1 incorporates the mutations T364E, F368Y, R460S, L464K, C467Y, and I424A relative to DDS5. E. coli strains expressing SQS1, SQE1 and Pq.DDS1 (or DDS5) were incubated at 37° C. for 72 hrs. The relative titer of strains expressing SQS1-SQE1-Pq.DDS1 is shown relative to strains expressing SQS1-SQE1-DDS5 (FIG. 3D). Pq.DDS1 leads to a nearly 20-fold improvement in dammarendiol titer.

Derivatives of Pq.DDS1 were created. Strains expressing SQS1, SQE1, and Pq.DDS1 derivatives were incubated at 37° C. for 72 hours. Dammarendiol levels were quantified by GC-FID chromatography using authentic standards. The fold improvement relative to Pq.DDS1 for each derivative is shown in Table 1. L195Del3 refers to the deletion of 3 residues (L195-E197) in Pq.DDS1.

The DDS1 derivative (Pq.DDS2) (SEQ ID NO: 81) was tested alongside Pq.DDS1. Pq.DDS2 incorporates the mutations Y49F, S181T, L195Del3, S198P, E238S, I407V, D507E, R637K, and M695I relative to Pq.DDS1. E. coli strains expressing SQS1, SQE1 and Pq.DDS2 (or Pq.DDS1) were incubated at 37° C. for 72 hrs. The relative titer of strains expressing SQS1-SQE1-Pq.DDS2 is shown relative to strains expressing SQS1-SQE1-Pq.DDS1 (FIG. 3E). Pq.DDS2 leads to an approximately 2-fold improvement in dammarenediol titer over Pq.DDS1.

Derivatives of Pq.DDS2 were created. Strains expressing SQS1, SQE1, and Pq.DDS2 derivatives were incubated at 37° C. for 72 hours. Dammarenediol levels were quantified by GC-FID chromatography using authentic standards. The fold improvement relative to Pq.DDS2 for each derivative is shown in Table 2.

The DDS1 derivative (Pq.DDS3) was tested alongside Pq.DDS2. Pq.DDS3 incorporates the mutations F649L, L548F, Q149E, A120S, G573A, S380A, and A256G relative to Pq.DDS2. E. coli strains expressing SQS1, SQE1 and Pq.DDS3 (or Pq.DDS2) were incubated at 37° C. for 72 hrs. The relative titer of strains expressing SQS1-SQE1-Pq.DDS3 is shown relative to strains expressing SQS1-SQE1-Pq.DDS2 (FIG. 3F). Pq.DDS3 leads to a greater than 2-fold improvement in dammarendiol titer over Pq.DDS2.

### Example 3. Production of Protopanaxadiol by the Microbial Strains

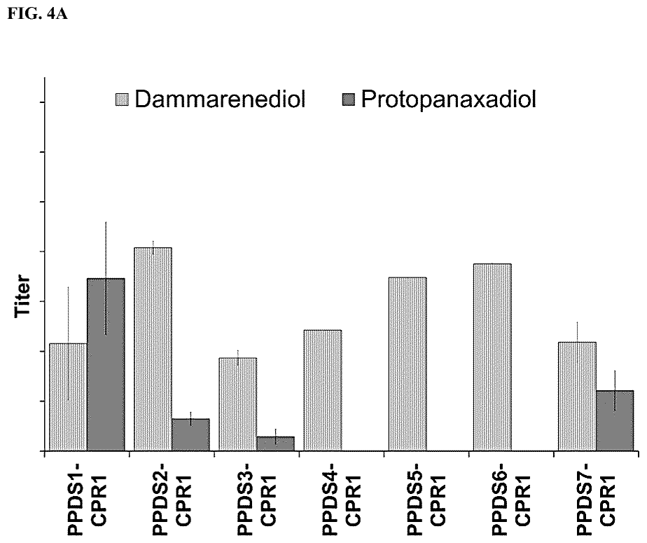

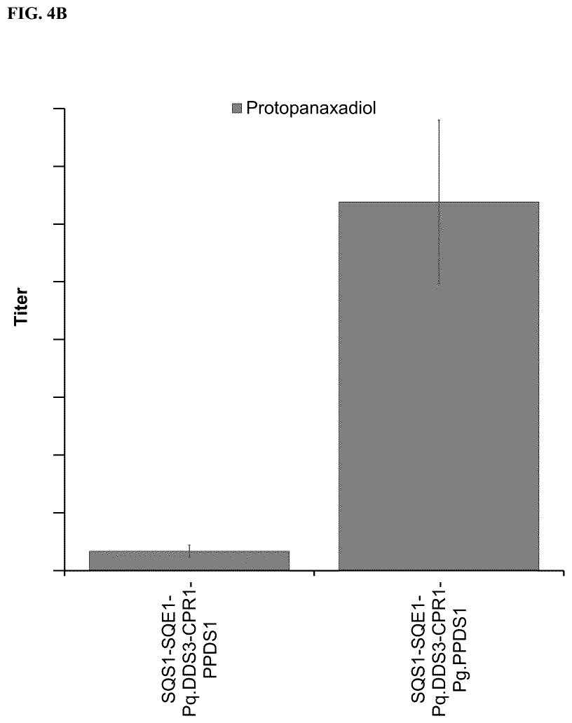

The strain producing dammarenediol (expressing DDS2) was further engineered to express a protopanaxadiol synthase (PPDS). Seven different PPDS enzymes PPDS1 to PPDS7 (SEQ ID NOs: 10-16) were expressed along with a cytochrome P450 reductase (CPR1). PPDS enzymes contained a truncation of the native N-terminus, which was replaced by an E. coli membrane anchor as described in U.S. Pat. No. 10,774,314, which is hereby incorporated by reference. The strains were incubated at 30° C. for 72 hr, and dammarenediol and protopanaxadiol were quantified by GC-FID chromatography using authentic standards of each compound. Titers of dammarenediol and protopanaxadiol produced by each strain were plotted. As shown in FIG. 4A, several strains expressing a PPDS produced protopanaxadiol. Productions of protopanaxadiol were verified by GC-MS spectrum analysis. PPDS1, PPDS2, PPDS3, and PPDS7 all demonstrated productions of protopanaxadiol. These results demonstrate that bacterial strains engineered to co-express a squalene synthase (SQS), a squalene monooxygenase (SQE), a dammarenediol synthase (DDS) and a protopanaxadiol synthase (PPDS) can produce protopanaxadiol.

To improve the production of protopanaxadiol, PPDS1 enzyme substitutions were screened, as shown in Table 3 (“PPDS1 derivatives”). Strains were incubated at 37° C. for 72 h, and protopanaxadiol levels were quantified by GC-FID chromatography using authentic standards. The fold improvement relative to PPDS1 (wild type) is shown in Table 3. A PPDS1 variant was produced (Pg.PPDS1) which incorporated the mutations T108N, I212F, K338G, D135E, S68P, V150P, F167H, L283M, H482R, R347Q, M390L, R243K, L292I, V329M, Q278E, and N58E relative to PPDS1. Strains expressing SQS1, SQE1, Pq.DDS3, CPR1, Pg.PPDS1 (or PPDS1) were incubated at 37° C. for 72 hrs. As shown in FIG. 4B, the strain expressing Pg.PPDS1 was approximately 20-fold better for producing protopanaxadiol than the strain expressing PPDS1.

### Example 4. Production of Protopanaxatriol by the Microbial Strains

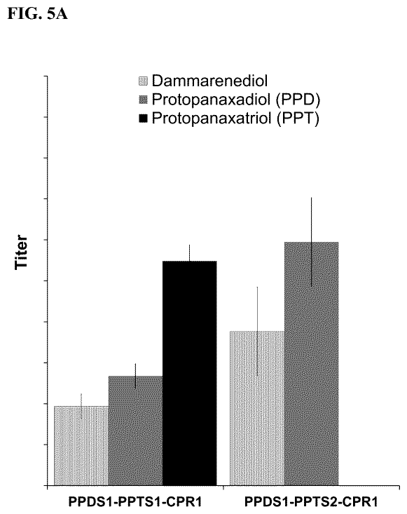

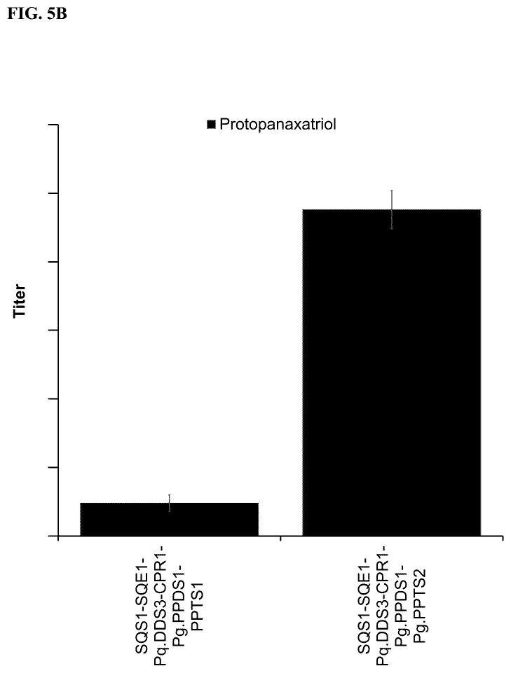

An E. coli strain producing protopanaxadiol was further engineered to express a protopanaxatriol synthase (PPTS). Like with PPDS, PPTS was engineered to include an N-terminal membrane anchor from an E. coli inner membrane protein. Two different PPTS enzymes PPTS1 (SEQ ID NO: 17) and PPTS2 (SEQ ID NO: 18) were expressed along with a cytochrome P450 reductase (CPR1). The strains expressing (1) PPDS1, CPR1 and PPTS1 and (2) PPDS1, CPR1 and PPTS2 were incubated at 30° C. for 72 hr. Dammarenediol, protopanaxadiol and protopanaxatriol were quantified by GC-FID chromatography using authentic standards of each compound and plotted. As shown in FIG. 5A, the strain expressing PPDS1, CPR1 and PPTS1 produced protopanaxatriol. Production of protopanaxatriol were verified by GC-MS spectrum analysis. These results demonstrate that a bacterial strain engineered to co-express a farnesyl diphosphate synthase (FPPS), a squalene synthase (SQS), a squalene epoxidase (SQE) and a dammarenediol synthase (DDS), a protopanaxadiol synthase (PPDS), and a protopanaxatriol synthase (PPTS) produced protopanaxatriol.

To improve the production of protopanaxatriol, PPTS1 enzyme was engineered by screening amino acid substitutions as shown in Table 4 (“PPTS1 derivatives”). Strains were incubated at 37° C. for 72 h, and protopanaxatriol levels were quantified by GC-FID chromatography using authentic standards. The fold improvements relative to PPTS1 (wild type) are shown in Table 4. A Pg.PPTS2 was created incorporated the mutations G294T, S166K, C472H, K252Q, V239I, A323P, I412V, I369T, K362D, and T250P relative to PPTS1. Strains expressing SQS1, SQE1, Pq.DDS3, CPR1, Pg.PPDS1, and Pg.PPTS2 (or PPTS1) were incubated at 37° C. for 72 hours. As shown in FIG. 5B, Pg.PPTS2 resulted in substantial improvements in protopanaxatriol relative titer (approximately 9-fold better than PPTS1).

SEQUENCES
Farnesyl Diphosphate Synthase
Saccharomyces cerevisiae FPPS
(SEQ ID NO: 80)
MASEKEIRRERFLNVFPKLVEELNASLLAYGMPKEACDWYAHSLNYNT
PGGKLNRGLSVVDTYAILSNKTVEQLGQEEYEKVAILGWCIELLQAYF
LVADDMMDKSITRRGQPCWYKVPEVGEIAINDAFMLEAAIYKLLKSHF
RNEKYYIDITELFHEVTFQTELGQLMDLITAPEDKVDLSKFSLKKHSF
IVTFKTAYYSFYLPVALAMYVAGITDEKDLKQARDVLIPLGEYFQIQD
DYLDCFGTPEQIGKIGTDIQDNKCSWVINKALELASAEQRKTLDENYG
KKDSVAEAKCKKIFNDLKIEQLYHEYEESIAKDLKAKISQVDESRGFK
ADVLTAFLNKVYKRSK
Squalene Synthases
Artemisia annua squalene synthase (SQS1)
(SEQ ID NO: 1)
MASSLKAVLKHPDDFYPLLKLKMAAKKAEKQIPSQPHWAFSYSMLHKV
SRSFALVIQQLNPQLRDAVCIFYLVLRALDTVEDDTSIAADIKVPILI
AFHKHIYNRDWHFACGTKEYKVLMDQFHHVSTAFLELKRGYQEAIEDI
TMRMGAGMAKFICKEVETVDDYDEYCHYVAGLVGIGLSKLFHSSGTEI
LFSDSISNSMGLFLQKTNIIRDYLEDINEIPKSRMFWPREIWSKYVNK
LEDLKYEENSEKAVQCLNDMVTNALIHIEDCLKYMSQLKDPAIFRFCA
IPQIMAIGTLALCYNNIEVFRGVVKLRRGLTAKVIDRTKTMADVYQAF
SDFSDMLKSKVDMHDPNAQTTITRLEAAQKICKDSGTLSNRKSYIVKR
ESSYSAALLALLFTILAILYAYLSANRPNKIKFTL
Siraitia grosvenorii SQSa
(SEQ ID NO: 23)
MGSLGAILRHPDDFYPLLKLKMAARHAEKQIPPEPHWGFCYTMLHKVS
RSFALVIQQLAPELRNAICIFYLVLRALDTVEDDTSIQTDIKVPILKA
FHCHIYNRDWHFSCGTKDYKVLMDQFHHVSTAFLELGKGYQEAIEDIT
KRMGAGMAKFICKEVETVDDYDEYCHYVAGLVGLGLSKLFHASDLEDL
APDSLSNSMGLLLQKTNIIRDYLEDINEIPKSRMFWPREIWGKYADKL
EDFKYEENSVKAVQCLNDLVTNALNHVEDCLKYMSNLRDLSIFRFCAI
PQIMAIGTLALCYNNVEVFRGVVKMRRGLTAKVIDRTQTMADVYGAFF
DFSVMLKAKVNSSDPNATKTLSRIEAIQKTCEQSGLLNKRKLYAVKSE
PMFNPTLIVILFSLLCIILAYLSAKRLPANQPV
Siraitia grosvenorii SQSb
(SEQ ID NO: 24)
MGSLGAILRHPDDFYPLLKLKMAARHAEKQIPPEPHWGFCYTMLHKVS
RSFALVIQQLAPELRNAICIFYLVLRALDTVEDDTSIQTDIKVPILKA
FHCHIYNRDWHFSCGTKDYKVLMDQFHHVSTAFLELGKGYQEAIEDIT
KRMGAGMAKFICKEVETVDDYDEYCHYVAGLVGLGLSKLFHASDLEDL
APDSLSNSMGLLLQKTNIIRDYLEDINEIPKSRMFWPREIWGKYADKL
EDFKYEENSVKAVQCLNDLVTNALNHVEDCLKYMSNLRDLSIFRFCAI
PQIMAIGTLALCYNNVEVFRGVVKMRRGLTAKVIDRTQTMADVYGAFF
DFSVMLKAKVNNSDPNATKTLSRIEAIQKTCEQSGLLNKRKLYAVKSE
PMFNPTLIVILFSLLCIILAYLSAKRLPANQPV
Cucumis sativus
(SEQ ID NO: 25)
MGSLGAILKHPDDFYPLLKLKIAARHAEKQIPPEPHWGFCYTMLHKVS
RSFALVIQQLKPELRNAVCIFYLVLRALDTVEDDTSIQTDIKVPILKA
FHCHIYNRDWHFSCGTKDYKVLMDEFHHVSTAFLELGKGYQEAIEDIT
KRMGAGMAKFICKEVETVDDYDEYCHYVAGLVGLGLSKLFHAAELEDL
APDSLSNSMGLFLQKTNIIRDYLEDINEIPKSRMFWPREIWGKYADKL
EDFKYEENSVKAVQCLNDLVTNALNHVEDCLKYMSNLRDLSIFRFCAI
PQIMAIGTLALCYNNVEVFRGVVKMRRGLTAKVIDRTKTMADVYGAFF
DFSVMLKAKVNSNDPNASKTLSRIEAIQKTCKQSGILNRRKLYVVRSE
PMFNPAVIVILFSLLCIILAYLSAKRLPANQSV
Cucumis melo
(SEQ ID NO: 26)
MGSLGAILKHPDDFYPLLKLKMAARHAEKQIPPESHWGFCYTMLHKVS
RSFALVIQQLKPELRNAVCIFYLVLRALDTVEDDTSIQTDIKVPILKA
FHCHIYNRDWHFSCGTKDYKVLMDEFHHVSTAFLELGKGYQEAIEDIT
KRMGAGMAKFICKEVETVDDYDEYCHYVAGLVGLGLSKLFHAAELEDL
APDSLSNSMGLFLQKTNIIRDYLEDINEIPKSRMFWPREIWGKYADKL
EDFKYEENSVKAVQCLNDLVTNALNHVEDCLKYMSNLRDLSIFRFCAI
PQIMAIGTLALCYNNVEVFRGVVKMRRGLTAKVIDRTKTMADVYGAFF
DFSVMLKAKVNSNDPNASKTLSRIEAIQQTCQQSGLMNKRKLYVVRSE
PMYNPAVIVILFSLLCIILAYLSAKRLPANQSV
Cucumis melo
(SEQ ID NO: 27)
MGSLGAILKHPDDFYPLLKLKMAARHAEKQIPPESHWGFCYTMLHKVS
RSFALVIQQLKPELRNAVCIFYLVLRALDTVEDDTSIQTDIKVPILKA
FHCHIYNRDWHFSCGTKDYKVLMDEFHHVSTAFLELGKGYQEAIEDIT
KRMGAGMAKFICKEVETVDDYDEYCHYVAGLVGLGLSKLFHAAELEDL
APDSLSNSMGLFLQKTNIIRDYLEDINEIPKSRMFWPREIWGKYADKL
EDFKYEENSVKAVQCLNDLVTNALNHVEDCPKYMSNLRDLSIFRFCAI
PQIMAIGTLALCYNNVEVFRGVVKMRRGLTAKVIDRTKTMADVYGAFF
DFSVMLKAKVNSNDPNASKTLSRIEAIQQTCQQSGLMNKRKLYVVRSE
PMYNPAVIVILFSLLCIILAYLSAKRLPANQSV
Cucurbita moschata
(SEQ ID NO: 28)
MGSLGAILRHPDDIYPLLKLKMAARHAEKQIPPESHWGFCYTMLHKVS
RSFALVIQQLKPELRNAVCIFYLVLRALDTVEDDTSIQTDIKVPILKA
FHCHIYNRDWHFSCGTKDYKVLMDEFHHVSTAFLELGRGYQEAIEDIT
KRMGAGMAKFICKEVETVEDYDEYCHYVAGLVGLGLSKLFHASKSENL
APDSLSNSMGLFLQKTNIIRDYLEDINEIPKSRMFWPREIWSKYADKL
EDFKYEKNSVKAVQCLNDLVTNALTHVEDCLEYMSNLKDLSIFRFCAI
PQIMAIGTLALCYNNVDVFRGVVKMRRGLTAKVIYRTKTMADVYGAFF
DFSVMLKAKVNSSDPNASKTLTRIEAIQKTCKQSGLLNKRELYAVRSE
PMCNPAAIVVLFSLLCIILAYLSAKLLPANQPV
Sechium edule
(SEQ ID NO: 29)
MGSLGAILSHPDDLYPLLKLKMAAKHAEKQIPPDPHWGFCFSMLHKVS
RSFALVIQQLKPELRNAVCIFYLVLRALDTVEDDTGIHPDIKVPILQA
FHCHIYNRDWHFSCGTKHYKVLMDEFHHVSTAFLELGKGYQEAIEDVT
ERMGAGMAKFICKEVETVDDYDEYCHYVAGLVGLGLSKLFHAAELEDL
APDSLSNSMGLFLQKTNIIRDYLEDINEIPKSRMFWPREIWNKYADKL
EDFKYEENSVKAVQCLNDLVTNALNHVEDCLKYMSNLKDLSTFRFCAI
PQIMAIGTLALCYDNVEVFRGVVKMRRGLTAKIIDRTKKIADVYGAFF
DFSVMLKAKVNSSDPNAAKTLSRIEAIEKTCKESGLLNKRKLYVIRSE
PLFNPAVLVILFSLICILLAYLSAKRLPANQPV
Panax quinquefolius
(SEQ ID NO: 30)
MGSLGAILKHPDDFYPLLKLKFAARHAEKQIPPEPHWAFCYSMLHKVS
RSFGLVIQQLGPQLRDAVCIFYLVLRALDTVEDDTSIPTEVKVPILMA
FHRHIYDKDWHFSCGTKEYKVLMDEFHHVSNAFLELGSGYQEAIEDIT
MRMGAGMAKFICKEVETIDDYDEYCHYVAGLVGLGLSKLFHASGAEDL
ATDSLSNSMGLFLQKTNIIRDYLEDINEIPKSRMFWPRQIWSKYVDKL
EDLKYEENSAKAVQCLNDMVTDALVHAEDCLKYMSDLRDPAIFRFCAI
PQIMAIGTLALCFNNTQVFRGVVKMRRGLTAKVIDRTKTMSDVYGAFF
DFSCLLKSKVDNNDPNATKTLSRLEAIQKTCKESGTLSKRKSYIIESE
SGHNSALIAIIFIILAILYAYLSSNLLLNKQ
Malus domestica
(SEQ ID NO: 31)
MGALSTMLKHPDDIYPLLKLKIASRQIEKQIPAEPHWAFCYTMLQKVS
RSFALVIQQLGTELRNAVCLFYLVLRALDTVEDDTSVATDVKVPILLA
FHRHIYDPDWHFACGTNNYKVLMDEFHHVSTAFLELGTGYQEAIEDIT
KRMGAGMAKFILKEVETIDDYDEYCHYVAGLVGLGLSKLFHAAGKEDL
ASDSLSNSMGLFLQKTNIIRDYLEDINEIPKSRMFWPRQIWSKYVNKL
EDLKYEENSEKAVQCLNDMVTNALIHMEDCLKYMAALRDPAIFKFCAI
PQIMAIGTLALCYNNIEVFRGVVKMRRGLTAKVIDRTKSMDDVYGAFF
DFSSILKSKVDKNDPNATKTLSRVEAVQKLCRDSGALSKRKSYIANRE
QSYNSTLIVALFIILAIIYAYLSASPRI
Glycine soja
(SEQ ID NO: 32)
MDQRSEDEFYPLLKLKIVARNAEKQIPPEPHWAFCYTMLHKVSRSFAL
VIQQLGIELRNAVCIFYLVLRALDTVEDDTSIETDVKVPILIAFHRHI
YDRDWHFSCGTKEYKVLMGQFHHVSTAFLELGKNYQEAIEDITKRMGA
GMAKFICKEVETIDDYDEYCHYVAGLVGLGLSKLFHASGSEDLAPDDL
SNSMGLFLQKTNIIRDYLEDINEIPKSRMFWPRQIWSEYVNKLEDLKY
EENSVKAVQCLNDMVTNALMHAEDCLTYMAALRDPPIFRFCAIPQIMA
IGTLALCYNNIEVFRGVVKMRRGLTAKVIDRTKTMADVYGAFFDFASM
LEPKVDKNDPNATKTLSRLEAIQKTCRESGLLSKRKSYIVNDESGYGS
TMIVILVIMVSIIFAYLSANHHNS
Diospyros kaki
(SEQ ID NO: 33)
MGSLAAMLRHPDDVYPLVKLKMAARHAEKQIPPEPHWAFCYTMLHKVS
RSFGLVIQQLGTELRNAVCIFYLVLRALDTVEDDTSIATEVKVPILLA
FHHHIYDRDWHFSCGTREYKVLMDEFHHVSTAFLELGKGYQEAIEDIT
MRMGAGMAKFICKEVETIDDYDEYCHYVAGLVGLGLSKLFHASGLEDL
APDSLSNSMGLFLQKTNIIRDYLEDINEIPKSRMFWPRQIWSKYVNKL
EDLKYEKNSVKSVQCLNDMVTNALIHVDDCLKYMSALRDPAIFRFCAI
PQIMAIGTLALCYNNIEVFRGVVKMRRGLTAKVIDQTKTISDVYGAFF
DFSCMLKSKVEKNDPNSTKTLSRIEAIQKTCRESGTLSKRKSYILRSK
RTHNSTLIFVLFIILAILFAYLSANRPPINM
Euphorbia lathyris
(SEQ ID NO: 34)
MGSLGAILKHPDDFYPLLKLKMAAKHAEKQIPAQPHWGFCYSMLHKVS
RSFSLVIQQLGTELRDAVCIFYLVLRALDTVEDDTSIPTDVKVPILIA
FHKHIYDPEWHFSCGTKEYKVLMDQIHHLSTAFLELGKSYQEAIEDIT
KKMGAGMAKFICKEVETVDDYDEYCHYVAGLVGLGLSKLFDASGFEDL
APDDLSNSMGLFLQKTNIIRDYLEDINEIPKSRMFWPRQIWSKYVNKL
EDLKYEENSVKAVQCLNDMVTNALIHMDDCLKYMSALRDPAIFRFCAI
PQIMAIGTLALCYNNVEVFRGVVKMRRGLTAKVIDRTRTMADVYRAFF
DFSCMMKSKVDRNDPNAEKTLNRLEAVQKTCKESGLLNKRRSYINESK
PYNSTMVILLMIVLAIILAYLSKRAN
Camellia oleifera
(SEQ ID NO: 35)
MGSLGAILKHPDDFYPLMKLKMAARRAEKNIPPEPHWGFCYSMLHKVS
RSFALVIQQLDTELRNAVCIFYLVLRALDTVEDDTSIATEVKVPILMA
FHRHIYDRDWHFSCGTKEYKVLMDEFHHVSTAFSELGRGYQEAIEDIT
MRMGAGMAKFICKEVETIDDYDEYCHYVAGLVGLGLSKLFHASGSEDL
ASDSLSNSMGLFLQVFLLTCIKTNIIRDYLEDINEIPKSRMFWPRQIW
SKYVNKLEDLKDKENSVKAVECLNDMVTNALIHVEDCLTYMSALRDPS
IFRFCAIPQIMAIGTLALCYNNIEVFRGVVKMRRGLTAKVIDRTKTMS
DVYGGFFDFSCMLKSKVNKSDPNAMKALSRLEAIQKICRESGTLNKRK
SYIIKSEPRYNSTLVFVLFIILAILFAYL
Eleutherococcus senticosus
(SEQ ID NO: 36)
MGSLGAILKHPDDFYPLLKLKFAARHAEKQIPPEPHWAFCYSMLHKVS
RSFGLVIQQLDAQLRDAVCIFYLVLRALDTVEDDTSIPTEVKVPILMA
FHRHIYDKDWHFSCGTKEYKVLMDEFHHVSNAFLELGSGFQEAIEDIT
MRMGAGMAKFICKEVETIDDYDEYCHYVAGLVGLGLSKLFHASGAEDL
ATDSLSNSMGLFLQKTNIIRDYLEDINEIPKSRMFWPRQIWSKYVDKL
ENLKYEENSAKAVQCLNDMVTNALLHAEDCLKYMSNLRDPAIFRFCAI
PQIMAIGTLALCFNNIQVFRGVVKMRRGLTAKVIDRTKTMSDVYGAFF
DFSCLLKSKVDNNDPNATKTLSRLEAIQKTCKESGTLSKRKSYIIESK
SAHNSALIAIIFIILAILYAYLSSNLPNNQ
Flavobacteriales bacterium
(SEQ ID NO: 37)
MLNNSLFSRLEEIPALLKLKLGSKDYYKNNNSETLTCDNLRYCFDTLN
KVSRSFATVIKQLPNELGNNVCVFYLILRALDSIEDDMNLPKELKIKL
LREFHKKNYESGWNISGVGDKKEHVELLENYDKVIQSFLAIDQKNQLI
ITDICRKVGAGMANFVKAEIESVEDYNLYCHHVAGLVGIGLSRMFISS
GLENDDFLNQDEISNSMGLFLQKTNIVRDYREDLDEGRMFWPKDIWHV
YGSKINDFAINPTHDQSVLCLNHMLNNALTHATDCLAYLKHLRNENIF
KFCAIPQVMAMATLCKIYSNPDVFIKNVKIRKGLAAKLILNTTSMDEV
IKVYKDMLLVIESKISSDNNPVSAETIQLLKQIREYFNDETLIVRKIA
Bacteroidetes bacterium
(SEQ ID NO: 38)
MLNSSLFSRLEEIPALLKLKLGSINNYKNNNSENLTSKNLRYCFDTLN
KVSRSFASVIKQLPNELMVNVCLFYLILRALDSIEDDMNLPKDFKINL
LREFLDKNYEPGWKISGVGDKKEYVELLENYDKVIQVFLDIDPKNQLI
ITDICRKMGAGMAHFVEAEINSVKDYNLYCYHVAGLVGIGLSKMFLAS
GLENCDYLNQEEISSSMGLFLQKTNIVRDYKEDMEENRIFWPKEIWRT
YASKFSDFSINPQHETSISCLNHMVNDALGHVIDCLEYLRHLRNENIF
KFCAIPQVMAMATLCKVYNNPDVFIKTVKIRKGLAAKLILNTTSMDEV
IKVYKGLLLDIENKIPLHNPTSDETLRLIKNIRSYCNNETMVVSKTA
Squalene Epoxidase
Methylomonas lenta (SQE1)
(SEQ ID NO: 2)
MAKEEFDICIIGAGMAGATISAYLAPKGIKIALIDHCYKEKKRIVGEL
LQPGAVLSLEQMGLSHLLDGFEAQTVKGYALLQGNEKTTIPYPSQHEG
IGLINGRFLQQIRASALENSSVTQIHGKALQLLENERNEIIGVSYRES
ITSQIKSIYAPLTITSDGFFSNFRAHLSNNQKTVTSYFIGLILKDCEM
PFPKHGHVFLSGPTPFICYPISDNEVRLLIDFPGEQLPRKNLLQEHLD
TNVTPYIPECMRSSYAQAIQEGGFKVMPNHYMAAKPIVRKGAVMLGDA
LNMRHPLTGGGLTAVFSDIQILSAHLLAMPDFKNTDLIHEKIEAYYRD
RKRANANLNILANALYAVMSNDLLKTAVFKYLQCGGANAQESIAVLAG
LNRKHFSLIKQFCFLAVFGACNLLQQSISNIPKALKLLKDAFVIIKPL
IKNELS
Siraitia grosvenorii SQE1
(SEQ ID NO: 39)
MVDQCALGWILASALGLVIALCFFVAPRRNHRGVDSKERDECVQSAAT
TKGECRFNDRDVDVIVVGAGVAGSALAHTLGKDGRRVHVIERDLTEPD
RIVGELLQPGGYLKLIELGLQDCVEEIDAQRVYGYALFKDGKNTRLSY
PLENFHSDVSGRSFHNGRFIQRMREKAASLPNVRLEQGTVTSLLEEKG
TIKGVQYKSKNGEEKTAYAPLTIVCDGCFSNLRRSLCNPMVDVPSYFV
GLVLENCELPFANHGHVILGDPSPILFYQISRTEIRCLVDVPGQKVPS
IANGEMEKYLKTVVAPQVPPQIYDSFIAAIDKGNIRTMPNRSMPAAPH
PTPGALLMGDAFNMRHPLTGGGMTVALSDIVVLRNLLKPLKDLSDAST
LCKYLESFYTLRKPVASTINTLAGALYKVFCASPDQARKEMRQACFDY
LSLGGIFSNGPVSLLSGLNPRPLSLVLHFFAVAIYGVGRLLLPFPSVK
GIWIGARLIYSASGIIFPIIRAEGVRQMFFPATVPAYYRSPPVFKPIV
Siraitia grosvenorii SQE2
(SEQ ID NO: 40)
MVDQCALGWILASVLGAAALYFLFGRKNGGVSNERRHESIKNIATTNG
EYKSSNSDGDIIIVGAGVAGSALAYTLGKDGRRVHVIERDLTEPDRIV
GELLQPGGYLKLTELGLEDCVDDIDAQRVYGYALFKDGKDTRLSYPLE
KFHSDVAGRSFHNGRFIQRMREKAASLPKVSLEQGTVTSLLEENGIIK
GVQYKTKTGQEMTAYAPLTIVCDGCFSNLRRSLCNPKVDVPSCFVGLV
LENCDLPYANHGHVILADPSPILFYRISSTEIRCLVDVPGQKVPSISN
GEMANYLKNVVAPQIPSQLYDSFVAAIDKGNIRTMPNRSMPADPYPTP
GALLMGDAFNMRHPLTGGGMTVALSDVVVLRDLLKPLRDLNDAPTLSK
YLEAFYTLRKPVASTINTLAGALYKVFCASPDQARKEMRQACFDYLSL
GGIFSNGPVSLLSGLNPRPISLVLHFFAVAIYGVGRLLIPFPSPKRVW
IGARIISGASAIIFPIIKAEGVRQMFFPATVAAYYRAPRVVKGR
Momordica charantia
(SEQ ID NO: 41)
MVDECALGWILAAALGAVIALCLFVAPKTNNQDGGVDSKATPECVQTT
NGECRSDGDSDVIIVGAGVAGSALAHTLGKDGRRVHVIERDLTEPDRI
VGELLQPGGYLKLIELGLADCVEEIDAQRVYGYALFKDGKNTRLSYPL
EKFHSDVSGRSFHNGRFIQRMREKADSLPNVRLEQGTVTSLLEEKGTI
KGVQYKSKDGKEKTAYAPLTIVCDGCFSNLRRSLCNPMVDVPSCFVGL
VLENCQLPFANHGHVVLGDPSPILFYPISSTEIRCLVDVPGQKVPSIS
NGEMEKYLKTVVAPQVPPQIYDAFIAAIDKGNIRTMPNRSMPAAPHPT
PGALLMGDAFNMRHPLTGGGMTVALSDIVVLRNLLKPLKDLHDAPTLC
KYLESFYTLRKPVASTINTLAGALYKVFCASPDQARKEMRQACFDYLS
LGGMFSNGPVSLLSGLNPRPLSLVLHFFAVAIYGVGRLLFPFPSPKGI
WIGARLIYSASGIIFPIIKAEGVRQMFFPATVPAYYRSPPALKPVA
Cucurbita maxima
(SEQ ID NO: 42)
MVDYCAFGWILAAVLGLAIALSFFVSPRRNRRGGADSTPRSEGVRSSS
TINGECRSVDGDADVIIVGAGVAGSALAHTLGKDGRLVHVIERDLTEP
DRIVGELLQPGGYLKLIELGLQDCVEEIDAQKVYGYALFKDGKNTQLS
YPLEKFQSDVSGRSFHNGRFIQRMREKAASLPNVRLEQGTVTSLLEEK
GTIKGVQYKSKNGEEKTAYAPLTIVCDGCFSNLRRSLCKPMVDVPSCF
VGLVLENCQLPFANHGHVVLGDPSPILFYPISSTEIRCLVDVPGQKIP
SISNGEMEKYLKTIVAPQVPPQIHDAFIAAIDKGNIRTMPNRSMPAAP
QPTPGALLMGDAFNMRHPLTGGGMTVALSDIVVLRNLLKPLKDLNDAP
TLCKYLESFYTLRKPVASTINTLAGALYKVFCASPDQARKEMRQACFD
YLSLGGIFSNGPVSLLSGLNPRPLSLVLHFFAVAIYGVGRLLLPFPSP
KGIWIGARLVYSASGIIFPIIKAEGVRQMFFPATVPAYYRSPPVHKSI
A
Cucurbita moschata
(SEQ ID NO: 43)
MVDYCAFGWILAAVLGLAIALSFFVSPRRNRRGGADSTPRSEGVRSSS
TTNGECRSVDCDADVIIVGAGVAGSALAHTLGKDGRLVHVIERDLTEP
DRIVGELLQPGGYLKLIELGLQDCVEEIDAQKVYGYALFKDGKNTQLS
YPLEKFQSDVSGRSFHNGRFIQRMREKAASLPNVRLEQGTVTSLLEEK
GTIKGVQYKSKNGEEKTAHAPLTIVCDGCFSNLRRSLCKPMVDVPSCF
VGLVLENCQLPFANHGHVVLGDPSPILFYPISSTEIRCLVDVPGQKVP
SISNGEMEKYLKTIVAPQVPPQIHDAFIAAIDKGNIRTMPNRSMPAAP
QPTPGALLMGDAFNMRHPLTGGGMTVALSDIVVLRNLLKPLKDLNDAP
TLCKYLESFYTLRKPVASTINTLAGALYKVFCASPDQARKEMRQACFD
YLSLGGIFSNGPVSLLSGLNPRPLSLVLHFFAVAIYGVGRLLLPFPSP
KGIWIGARLVYSASGIIFPIIKAEGVRQMFFPATVPAYYRSPPVLKTI
A
Cucurbita moschata
(SEQ ID NO: 44)
MMVDHCAFAWILDVVLGLVVAVTFFVAAPRRNRRGGTDSTASKDCVIS
TAIANGECKPDDADAEVIIVGAGVAGSALAYTLGKDGRRVHVIERDLT
EPDRIVGEFLQPGGYLKLIELGLGDCVEEIDAQKLYGYALFKDGKNTR
VSYPLGNFHSDVSGRSFHNGRFIQRMREKAASLPNVRLEQGTVTSLLE
TKGTIKGVQYKSKNGEEKTAYAPLTIVCDGCFSNLRRSLCKPMVDVPS
CFVGLVLENCQLPFANHGHVVLGDPSPILFYPISSTEIRCLVDVPGQK
VPSISNGDMEKYLKTVVAPQVPPQIHDAFIAAIEKGNVRTMPNRSMPA
APHPTPGALLMGDAFNMRHPLTGGGMTVALSDIVVLRNLLKPLKDLND
ASTLCKYLESFYTLRKPVASTINTLAGALYKVFCASPDQARKEMRQAC
FDYLSLGGVFSNGPISLLSGLNPRPSSLVLHFFAVAIYGVGRLLLPFP
SLKGIWIGARLIYSASGIILPIIKAEGVRQMFFPATVPAYYRSPPVHK
PIT
Cucumis sativus
(SEQ ID NO: 45)
MVDHCTFGWIFSAFLAFVIAFSFFLSPRKNRRGRGTNSTPRRDCLSSS
ATTNGECRSVDGDADVIIVGAGVAGSALAHTLGKDGRRVHVIERDLTE
PDRIVGELLQPGGYLKLIELGLQDCVEEIDAQKVYGYALFKDGKSTRL
SYPLENFQSDVSGRSFHNGRFIQRMREKAAFLPNVRLEQGTVTSLLEE
KGTITGVQYKSKNGEQKTAYAPLTIVCDGCFSNLRRSLCNPMVDVPSC
FVGLVLENCQLPYANLGHVVLGDPSPILFYPISSTEIRCLVDVPGQKV
PSISNGEMEKYLKTVVAPQVPPQIHDAFIAAIEKGNIRTMPNRSMPAA
PQPTPGALLMGDAFNMRHPLTGGGMTVALSDIVVLRNLLKPLKDLNDA
PTLCKYLESFYTLRKPVASTINTLAGALYKVFCASSDQARKEMRQACF
DYLSLGGIFSNGPVSLLSGLNPRPLSLVLHFFAVAIYGVGRLLLPFPS
PKGIWIGARLVYSASGIIFPIIKAEGVRQMFFPATVPAYYRTPPVENS
Cucumis melo
(SEQ ID NO: 46)
MVDHCAFGWIFSALLAFPIALSLFLSPWRNRRVRGTDSTPRSASVSSS
ATTNGECRSVDGDADVVIVGAGVAGSALAHTLGKDGRRVHVIERDLTE
PDRIVGELLQPGGYLKLIELGLQDCVEEIDAQKVYGYALFKDGKNTRL
SYPLENFHSDVSGRSFHNGRFIQRMREKAASLPNVRLEQGTVTSLLEE
KGTITGVQYKSKNGEQKTAYAPLTIVCDGCFSNLRRSLCTPMVDVPSY
FVGLVLENCQLPYANLGHVVLGDPSPILFYPISSTEIRCLVDVPGQKV
PSISNGEMEKYLKTVVAPQVPPQIHDAFIAAIEKGNIRTMPNRSMPAA
PQPTPGALLMGDAFNMRHPLTGGGMTVALSDIVVLRNLLKPLKDLNDA
PTLCKYLESFYTLRKPVASTINTLAGALYKVFCASPDQARKEMRQACF
DYLSLGGIFSNGPVSLLSGLNPRPLSLVLHFFAVAIYGVGRLLLPFPS
LKGIWIGARLVYSASGIIFPIIKAEGVRQMFFPATVPAYYRTPPVLNS
Cucurbita maxima
(SEQ ID NO: 47)
MMVEHCAYGWILAAVLGLVVAVTFFVAVPRRNRRGGTDSTASKDCVIS
PAIANGECEPEDADADADVIIVGAGVAGSALAHTLGKDGRRVHVIERD
LTEPDRIVGEFLQPGGHLKLIELGLGDCVEEIDAQKLYGYALFKDGKN
TRVSYPLGNFHSDVSGRSFHNGRFIQRMREKAASLPNVRLEQGTVTSL
LEKKGTIKGVQYKSKNGEEKTAYAPLTIVCDGCFSNLRRSLCKPMVDV
PSCFVGLVLENCRLPFANHGHVVLGDPSPILFYPISSTEIRCLVDVPG
QKVPSIPNGDMEKYLKTVVAPQVPPQIHDAFIAAIEKGNIRTMPNRSM
PAAPHPTPGALLMGDAFNMRHPLTGGGMTVALSDIVVLRNLLKPLKDL
NDAPTLCKYLESYYTLRKPVASTINTLAGALYKVFCASPDQARKEMRQ
ACFDYLSLGGVFSNGPISLLSGLNPRPSCLVLHFFAVAIYGVGRLLLP
FPSLKGIWIGARLIYSASGIILPIIKAEGVRQMFFPATVPAYYRSPPV
HKPIT
Ziziphus jujube
(SEQ ID NO: 48)
MLDQCPLGWILASVLGLFVLCNLIVKNRNSKASLEKRSECVKSIATTN
GECRSKSDDVDVIIVGAGVAGSALAHTLGKDGRRLHVIERDLTEPDRI
VGELLQPGGYLKLIELGLQDCVEEIDAQRVFGYALFKDGKDTRLSYPL
EKFHSDVSGRSFHNGRFIQRMREKSASLPNVRLEQGTVTSLLEEKGTI
KGVQYKTKTGQELTAFAPLTIVCDGCFSNLRRSLCNPKVDVPSCFVGL
VLENCELPYANHGHVILADPSPILFYPISSTEVRCLVDVPGQKVPSIS
NGEMAKYLKSVVAPQIPPQIYDAFIAAVDKGNIRTMPNRSMPASPFPT
PGALLMGDAFNMRHPLTGGGMTVALSDIVVLRDLLKPLGDLNDAATLC
KYLESFYTLRKPVASTINTLAGALYKVFCASPDQARKEMRQACFDYLS
LGGIFSTGPVSLLSGLNPRPLSLVLHFFAVAIYGVGRLLLPFPSPKRI
WIGARLISGASGIIFPIIKAEGVRQMFFPATVPAYYRAAPVE
Morus alba
(SEQ ID NO: 49)
MADPYTMGWILASLLGLFALYYLFVNNKNHREASLQESGSECVKSVAP
VKGECRSKNGDADVIIVGAGVAGSALAHTLGKDGRRVHVIERDLAEPD
RIVGELLQPGGYLKLIELGLQDCVEEIDSQRVYGYALFKDGKDTRLSY
PLEKFHSDVSGRSFHNGRFIQRMREKAASLPNVQLEQGTVTSLLEENG
TIKGVQYKTKTGQELTAYAPLTIVCDGCFSNLRRSLCIPKVDVPSCFV
GLVLENCNLPYANHGHVVLADPSPILFYPISSTEVRCLVDVPGQKVPS
ISNGEMAKYLKTVVASQIPPQIYDSFVAAVDKGNIRTMPNRSMPAAPH
PTPGALLMGDAFNMRHPLTGGGMTVALSDIVVLRDLLKPLRDLNDSVT
LCKYLESFYTLRKPVASTINTLAGALYKVFCASPDQARKEMREACFDY
LSLGGVFSEGPVSLLSGLNPRPLSLVCHFFAVAIYGVGRLLLPFPSPK
RLWIGARLISGASGIIFPIIRAEGVRQMFFPATIPAYYRAPRPN
Juglans regia (JrSQE1)
(SEQ ID NO: 50)
MVDPYALGWSFASVLMGLVALYILVDKKNRSRVSSEARSEGVESVTTT
TSGECRLTDGDADVIIVGAGVAGSALAHTLGKDGRRVHVIERDLTEPD
RIVGELLQPGGYLKLIELGLEDCVEDIDAQRVFGYALFKDGKNTRLSY
PLEKFHSDVSGRSFHNGRFIQRMREKAASLLNVRLEQGTVTSLLEENG
TVKGVQYKTKDGNELTAHAPLTIVCDGCFSNLRRSLCNPQVDVPSSFV
GLVLENCELPYANHGHVILADPSPILFYPISSTEVRCLVDVPGKKVPS
IANGEMEKYLKNMVAPQLPPEIYDSFVAAVDRGNIRTMPNRSMPAAPH
PTPGALLMGDAFNMRHPLTGGGMTVALSDIVVLRDLLKPLRDLNDAPT
LCKYLESFYTLRKPVASTINTLAGALYKVFCASPDRARKEMRQACFDY
LSLGGVFSMGPVSLLSGLNPRPLSLVLHFFAVAVYGVGRLLVPFPSPS
RIWIGARLISGASAIIFPIIKAEGVRQMFFPATVPAYYRAPPVKRDH
Cucumis melo
(SEQ ID NO: 51)
MVDQCALGWILASVLGASALYLLFGKKNCGVLNERRRESLKNIATTNG
ECKSSNSDGDIIIVGAGVAGSALAYTLAKDGRQVHVIERDLSEPDRIV
GELLQPGGYLKLTELGLEDCVDDIDAQRVYGYALFKDGKDTRLSYPLE
KFHSDVSGRSFHNGRFIQRMREKAASLPNVRLEQGTVTSLLEENGTIK
GVQYKNKSGQEMTAYAPLTIVCDGCFSNLRRSLCNPKVDVPSCFVGLI
LENCDLPYANHGHVILADPSPILFYPISSTEIRCLVDVPGQKVPSISN
GEMANYLKNVVAPQIPPQLYNSFIAAIDKGNIRTMPNRSMPADPYPTP
GALLMGDAFNMRHPLTGGGMTVALSDIVVLRDLLKPLRDLNDAPTLCK
YLEAFYTLRKPVASTINTLAGALYKVFCASPDQARKEMRQACFDYLSL
GGIFSNGPVSLLSGLNPRPLSLVLHFFAVAIYGVGRLLIPFPSPKRVW
IGARLISGASAIIFPIIKAEGVRQMFFPKTVAAYYRAPPVVRER
Cucumis sativus
(SEQ ID NO: 52)
MVDQCALGWILASVLGASALYLLFGKKNCGVSNERRRESLKNIATTNG
ECKSSNSDGDIIIVGAGVAGSALAYTLAKDGRQVHVIERDLSEPDRIV
GELLQPGGYLKLTELGLEDCVDEIDAQRVYGYALFKDGKDTRLSYPLE
KFHSDVSGRSFHNGRFIQRMREKAASLPNVRLEQGTVTSLLEENGTIR
GVQYKNKSGQEMTAYAPLTIVCDGCFSNLRRSLCNPKVDVPSCFVGLI
LENCDLPHANHGHVILADPSPILFYPISSTEIRCLVDVPGQKVPSISN
GEMANYLKNVVAPQIPPQLYNSFIAAIDKGNIRTMPNRSMPADPYPTP
GALLMGDAFNMRHPLTGGGMTVALSDIVVLRDLLKPLRDLNDAPTLCK
YLEAFYTLRKPVASTINTLAGALYKVFCASPDQARKEMRQACFDYLSL
GGIFSNGPVSLLSGLNPRPLSLVLHFFAVAIYGVGRLLIPFPSPKRVW
IGARLISGASAIIFPIIKAEGVRQMFFPKTVAAYYRAPPIVRER
Juglans regia (JrSQE2)
(SEQ ID NO: 53)
MVDQYALGLI
LASVLGFVVLYNLMAKKNRIRVSSEARTEGVQTVITTINGECRSIEG
DVDVIIVGAGVAGSALAHTLGKDGRKVHVIERDLSEPDRIVGELLQPG
GYLKLVELGLQDSVEDIDAQRVFGYALFKDGKNTRLSYPLEKFHSDVS
GRSFHNGRFIQRMREKAASLPNIRLEQGTVTSLLEENGTIKGVQYKTK
DGKELAAHAPLTIVCDGCFSNLRRSLCNPQVDVPSSFVGLVLENCELP
YANHGHVVLADPSPILFYPISSTEVRCLVDVPGQKVPSISNGEMAKYL
KTMVAPQVPPEIYDSFVAAVDRGNIRTMPNRSMPAAPQPTPGALLMGD
AFNMRHPLTGGGMTVALSDIVVLRDLLRPLRDLNDAPTLCKYLESFYT
LRKPVASTINTLAGALYKVFCASPDRARNEMRQACFDYLSLGGVFSTG
PVSLLSGLNPRPLSLVLHFFAVAVYGVGRLLVPFPSPSRMWIGARLIS
GASAIIFPIIKAEGVRQMFFPATVPAYYRAPPVNCQARSLKPDALKGL
Theobroma cacao
(SEQ ID NO: 54)
MADSYVWGWILGSVMTLVALCGVVLKRRKGSGISATRTESVKCVSSIN
GKCRSADGSDADVIIVGAGVAGSALAHTLGKDGRRVHVIERDLTEPDR
IVGELLQPGGYLKLIELGLEDCVEEIDAQQVFGYALFKDGKHTRLSYP
LEKFHSDVSGRSFHNGRFIQRMREKSASLPNVRLEQGTVTSLLEEKGT
IRGVQYKTKDGRELTAFAPLTIVCDGCFSNLRRSLCNPKVDVPSCFVG
LVLENCNLPYSNHGHVILADPSPILFYPISSTEVRCLVDVPGQKVPSI
ANGEMANYLKTIVAPQVPPEIYNSFVAAVDKGNIRTMPNRSMPAAPYP
TPGALLMGDAFNMRHPLTGGGMTVALSDIVVLRDLLRPLRDLNDAPTL
CKYLESFYTLRKPIASTINTLAGALYKVFCASPDQARKEMRQACFDYL
SLGGVFSTGPISLLSGLNPRPVSLVLHFFAVAIYGVGRLLLPFPSPKR
IWIGARLISGASGIIFPIIKAEGVRQMFFPATVPAYYRAPPVE
Cucurbita moschata
(SEQ ID NO: 55)
MMVDHCAFAWILDVVLGLVVAVTFFVAAPRRNRRGGTDSTASKDCVIS
TAIANGECKPDDADAEVIIVGAGVAGSALAYTLGKDGRRVHVIERDLT
EPDRIVGEFLQPGGYLKLIELGLGDCVEEIDAQKLYGYALFKDGKNTR
VSYPLGNFHSDVSGRSFHNGRFIQRMREKAASLPNVRLEQGTVTSLLE
TKGTIKGVQYKSKNGEEKTAYAPLTIVCDGCFSNLRRSLCKPMVDVPS
CFVGLVLENCQLPFANHGHVVLGDPSPILFYPISSTEIRCLVDVPGQK
VPSISNGDMEKYLKTVVAPQVPPQIHDAFIAAIEKGNVRTMPNRSMPA
APHPTPGALLMGDAFNMRHPLTGGGMTVALSDIVVLRNLLKPLKDLND
ASTLCKYLESFYTLRKPVASTINTLAGALYKVFCASPDQARKEMRQAC
FDYLSLGGVFSNGPISLLSGLNPRPSSLVLHFFAVAIYGVGRLLLPFP
SLKGIWIGARLIYSASGIILPIIKAEGVRQMFFPATVPAYYRSPPVHK
PIT
Phaseolus vulgaris
(SEQ ID NO: 56)
MLDTYVFGWIICAALSVFVIRNFVFAGKKCCASSETDASMCAENITTA
AGECRSSMRDGEFDVLIVGAGVAGSALAYTLGKDGRQVLVIERDLSEP
DRIVGELLQPGGYLKLIELGLEDCVDKIDAQQVFGYALFKDGKHIRLS
YPLEKFHSDVAGRSFHNGRFIQRMREKAASLPNVRLEQGTVTSLLEEK
GVIKGVQYKTKDSQELSVCAPFTIVCDGCFSNLRRSLCDPKVDVPSCF
VGLVLENCELPCANHGHVILGEPSPVLFYPISSTEIRCLVDVPGQKVP
SISNGEMAKYLKTVIAPQVPHELHNAFIAAVDKGSIRTMPNRSMPAAP
YPTPGALLMGDAFNMRHPLTGGGMTVALSDIVVLRNLLRPLRDLNDAP
SLCKYLESFYTLRKPVASTINTLAGALYKVFCASSDPARKEMRQACFD
YLSLGGQFSEGPISLLSGLNPRPLTLVLHFFAVATYGVGRLLLPFPSP
KRMWIGLRLISSASGIIMPIIKAEGVRQMFFPATVPAYYRNPPAA
Hevea brasiliensis
(SEQ ID NO: 57)
MKMADHYLLGWILASVMGLFAFYYIVYLLVKPEEDNNRRSLPQPRSDF
VKTMTATNGECRSDDDSDVDVIIVGAGVAGAALAHTLGKDGRRVHVIE
RDLTEPDRIVGELLQPGGYLKLIELGLEDCVEEIDAQRVFGYALFKDG
KHTQLAYPLEKFHSEVAGRSFHNGRFIQRMREKAASLPSVKLEQGTVT
SLLEEKGTIKGVLYKTKTGEELTAFAPLTIVCDGCFSNLRRSLCNPKV
DVPSCFVGLVLENCRLPYANNGHVILADPSPILFYPISSTEVRSLVDV
PGQKVPSVSSGEMANYLKNVVAPQVPPEIYDSFVAAVDKGNIRTMPNR
SMPASPYPTPGALLMGDAFNMRHPLTGGGMTVALSDIVVLRDLLKPLR
DLHDAPTLCRYLESFYTLRKPVASTINTLAGALYKVFCASPDEARKEM
RQACFDYLSLGGVFSTGPVSLLSGLNPRPLSLVLHFFAVAIYGVGRLL
LPFPSPHRIWVGARLISGASGIIFPIIKAEGVRQMFFPATVPAYYRAP
PIKCN
Sorghum bicolor
(SEQ ID NO: 58)
MAAAAAAASGVGFQLIGAAAATLLAAVLVAAVLGRRRRRARPQAPLVE
AKPAPEGGCAVGDGRTDVIIVGAGVAGSALAYTLGKDGRRVHVIERDL
TEPDRIVGELLQPGGYLKLIELGLEDCVEEIDAQRVLGYALFKDGRNT
KLAYPLEKFHSDVAGRSFHNGRFIQRMRQKAASLPNVQLEQGTVTSLL
EENGTVKGVQYKTKSGEELKAYAPLTIVCDGCFSNLRRALCSPKVDVP
SCFVGLVLENCQLPHPNHGHVILANPSPILFYPISSTEVRCLVDVPGQ
KVPSIASGEMANYLKTVVAPQIPPEIYDSFIAAIDKGSIRTMPNRSMP
AAPHPTPGALLMGDAFNMRHPLTGGGMTVALSDIVVLRNLLKPLHNLH
DASSLCKYLESFYTLRKPVASTINTLAGALYKVFSASPDQARNEMRQA
CFDYLSLGGVFSNGPIALLSGLNPRPLSLVAHFFAVAIYGVGRLMLPL
PSPKRMWIGARLISGACGIILPIIKAEGVRQMFFPATVPAYYRAAPMG
E
Zea mays
(SEQ ID NO: 59)
MRKNLEEAGCAVSDGGTDVIIVGAGVAGSALAYTLGKDGRRVHVIERD
LTEPDRIVGELLQPGGYLKLIELGLQDCVEEIDAQRVLGYALFKDGRN
TKLAYPLEKFHSDVAGRSFHNGRFIQRMRQKAASLPNVQLEQGTVTSL
LEENGTVKGVQYKTKSGEELKAYAPLTIVCDGCFSNLRRALCSPKVDV
PSCFVGLVLENCQLPHPNHGHVILANPSPILFYPISSTEVRCLVDVPG
QKVPSIATGEMANYLKTVVAPQIPPEIYDSFIAAIDKGSIRTMPNRSM
PAAPHPTPGALLMGDAFNMRHPLTGGGMTVALSDIVVLRNLLKPLRNL
HDASSLCKYLESFYTLRKPVASTINTLAGALYKVFSASPDQARNEMRQ
ACFDYLSLGGVFSNGPIALLSGLNPRPLSLVAHFFAVAIYGVGRLMLP
LPSPKRMWIGARLISGACGIILPIIKAEGVRQMFFPATVPAYYRAAPT
GEKA
Medicago sativa
(SEQ ID NO: 60)
MDLYNIGWILSSVLSLFALYNLIFSGKRNYHDVNDKVKDSVTSTDAGD
IQSEKLNGDADVIIVGAGIAGAALAHTLGKDGRRVHIIERDLSEPDRI
VGELLQPGGYLKLVELGLQDCVDNIDAQRVFGYALFKDGKHTRLSYPL
EKFHSDVSGRSFHNGRFIQRMREKAASLPNVNMEQGTVISLLEEKGTI
KGVQYKNKDGQALTAYAPLTIVCDGCFSNLRRSLCNPKVDNPSCFVGL
ILENCELPCANHGHVILGDPSPILFYPISSTEIRCLVDVPGTKVPSIS
NGDMTKYLKTTVAPQVPPELYDAFIAAVDKGNIRTMPNRSMPADPRPT
PGAVLMGDAFNMRHPLTGGGMTVALSDIVVLRNLLKPMRDLNDAPTLC
KYLESFYTLRKPVASTINTLAGALYKVFSASPDEARKEMRQACFDYLS
LGGLFSEGPISLLSGLNPRPLSLVLHFFAVAVFGVGRLLLPFPSPKRV
WIGARLLSGASGIILPIIKAEGIRQMFFPATVPAYYRAPPVNAF
Bathymodiolus azoricus Endosymbiont
(SEQ ID NO: 61)
MHTTSEHNDLFDICIVGAGMAGATIATYLAPRGIKIALIDRDYAEKRRI
VGELLQPGAVQTLKKMGLEHLLEGFDAQPIYGYALFNKDCEFSIEYNQ
DKSTNYRGVGLHNGRFLQKIREDALKQPSITQIHGTVSELIEDENHVV
TGVKYKEKYTRELKTVNAKLTITSDGFFSSFRKDLTNNVKTVTSFFVG
IILKDCELPYPHHGHVFLSAPTPFICYPISSTESRLLIDFPGDQAPKK
EAVKHHIENNVIPFLPKEFRLCLDQALRENDYKIMPNHYMPAKPVLKK
GVVLLGDALNMRHPITGGGLTAVFNDVYLLSTHLLAMPDENDTKLIHE
KVNLYYNDRYHANTNVNIMANALYGVMSNDLLKQSVFEYLRKGGDNSG
GPISLLAGLNRNPTILIKHFFSVALLCLRNLFKAHKMSLTNAFYVIKD
AFCIIVPLAINELRPSSFLKKNIHN
Methyloprofundus sediment
(SEQ ID NO: 62)
MNTSPEHNDLFDICIVGVGMAGATIAAYLAPRGLKIALIDREYTEKRR
IVGELLQPGAVQTLKKMGLEHLLEGFDAQPIYGYALFNNDKEFSISYN
SDDSTEYHGVGLHNGRFLQKIREDVFKNETVTQIHGTVSELIEDKKGV
VKGVTYREKHTREYKTVKAKLTVTSDGFFSNFRKDLSNNVKTVTSFFI
GLVLNDCNLPFPNHGHVFLSAPTPFICYPISSTETRLLIDYPGDKAPK
KDEIREHILNKVAPFLPEEFKECFANAMEDDDFKVMPNHYMPAKPVLK
EGAVLLGDALNMRHPLTGGGLTAVFNDVYLLSTHLLAMPDFNDPKLLH
EKLELYYQDRYHANTNVNIMANALYGVMSNDLLKQGVFEYLRKGGDNS
GGPITLLAGLNRNPTLLIKHFFSVAFLCICNLSGNNKMNFTNVFRVMK
DAFCIIKPLAVNELRPSSFYKKNIQL
Methylomicrobium buryatense
(SEQ ID NO: 63)
MESNEDICIIGAGMAGATIAAYLAPKGINIALIDHCYKEKKRIVGELL
QPGAVLSLEQLGLGHLLDGIDAQPVEGYALLQGNEQTTIPYPSPNHGM
GLHNGRFLQQIRASALQNSSVTQIQGKALSLLENEQNEIIGVNYRDSV
SNEIKSIYAPLTITSDGFFSNFRELLSNNEKTVTSYFIGLILKDCEIP
VPKHIGHVFLSGPTPFICYPISSNEVRLLIDFPGGQFPRKAFLQAHLE
TNVTPYIPEGMQTSYRHALQEDRLKVMPNHYMAAKPKIRKGAVMLGDA
LNMRHPLTGGGLTAVFSDIEILSGHLLAMPDFNNNDLIYQKIEAYYRD
RQYANANLNILANALYGVMSNELLKNSVFKYLQRGGVNAKESIAILAG
LNKNHYSLMKQFFFVALFGAYTLVRENITNLPKATKILSDALTIIKPL
AKNELSLVGIFSDYFKR
Ononis spinosa SQEL
(SEQ ID NO: 64)
MVDPYAVGWIICSLTTIVALYNFVFYRQNRSDKTTPTTTENITTATGD
CRSLNPNGDVDIVIVGAGVAGSALAYTLGKDGRRVLVIERDLNEPDRI
VGELLQPGGYLKLIELGLEDCVEKIDAQQVFGYALFKDGKHTRLSYPL
EKFHSDIAGRSFHNGRFIQRMREKAASLPNVQLVQGTVTSLLEENGTI
KGVQYKTKDAQELSACAPLTIVCDGCFSNLRRNLCNPKVEVPSCFVGL
VLENCELPCANHGHVILGDPSPVLFYPISSTEIRCLVDVPGQKVPSIS
NGEMAKYLKEVVAPQVPPELHDAFIAAVDKGNIRTMPNRSMPAAPYPT
PGALLMGDAFNMRHPLTGGGMTVALSDIVVLRNLLKPLRDLNDAPSLC
KYLESFYTLRKPVASTINTLAGALYKVFCASPDPARKEMRQACFDYLS
LGGLFSEGPVSLLSGLNPRPLSLVLHFFAVAIYGVGRLLLPFPSPKRI
WIGVRLIASASGIILPIIKAEGIRQMFFPATVPAYYRTPPAA
Ononis spinosa SQE2
(SEQ ID NO: 65)
MDLYLLGWILSSVLSLFALYCLVFDGNRSRANAEKQIQRGYSVTTDAG
DVKSEKLNGDADVIIVGAGIAGAALAHTLGKDGRRVRVIERDLSEPDR
IVGELLQPGGYLKLVELGLADCVDNIDAQKVFGYALFKDGKHTRLSYP
LEKFHADVSGRSFHNGRFIQRMREKAASLLNVNLEQGTVTSLLEEKGT
IKGVQYKNKDGQELTAYAPLTIVCDGCFSNLRRSLCNPKVDNPSCFVG
LVLENCELPCANHGHVILGDPSPILFYPISSTEIRCLVDVPGQKVPSI
SNGDMTKYLKLTVAPQVPPELYDAFIAAVDKGNIRTMPNKSMPADPCP
TPGAVLMGDAFNMRHPLTGGGMTVALSDIVVLRNLLRPLRDLNDAPAL
CKYLESFYTLRKPVASTINTLAGALYKVFSSSPDQARREMRQACFDYL
SLGGLFSEGPISLLSGLNPRPLSLVLHFFAVAVFGVGRLLLPFPSPKR
VWIGARLLSAASGIILPIIKAEGIRQMFFPVTVPAYYRAPPTSQE
Medicago truncatula SQE1
(SEQ ID NO: 66)
MIDPYGFGWITCTLITLAALYNFLFSRKNHSDSTTTENITTATGECRS
FNPNGDVDIIIVGAGVAGSALAYTLGKDGRRVLIIERDLNEPDRIVGE
LLQPGGYLKLIELGLDDCVEKIDAQKVFGYALFKDGKHTRLSYPLEKF
HSDIAGRSFHNGRFILRMREKAASLPNVRLEQGTVTSLLEENGTIKGV
QYKTKDAQEFSACAPLTIVCDGCFSNLRRSLCNPKVEVPSCFVGLVLE
NCELPCADHGHVILGDPSPVLFYPISSTEIRCLVDVPGQKVPSISNGE
MAKYLKTVVAPQVPPELHAAFIAAVDKGHIRTMPNRSMPADPYPTPGA
LLMGDAFNMRHPLTGGGMTVALSDIVVLRNLLKPLRDLNDASSLCKYL
ESFYTLRKPVASTINTLAGALYKVFCASPDPARKEMRQACFDYLSLGG
LFSEGPVSLLSGLNPCPLSLVLHFFAVAIYGVGRLLLPFPSPKRLWIG
IRLIASASGIILPIIKAEGIRQMFFPATVPAYYRAPPDA
Medicago truncatula SQE2
(SEQ ID NO: 67)
MDLYNIGWILSSVLSLFALYNLIFAGKKNYDVNEKVNQREDSVTSTDA
GEIKSDKLNGDADVIIVGAGIAGAALAHTLGKDGRRVHIIERDLSEPD
RIVGELLQPGGYLKLVELGLQDCVDNIDAQRVFGYALFKDGKHTRLSY
PLEKFHSDVSGRSFHGRFIQRMREKAASLPNVNMEQGTVISLLEEKGT
IKGVQYKNKDGQALTAYAPLTIVCDGCFSNLRRSLCNPKVDNPSCFVG
LILENCELPCANHGHVILGDPSPILFYPISSTEIRCLVDVPGTKVPSI
SNGDMTKYLKTTVAPQVPPELYDAFIAAVDKGNIRTMPNRSMPADPRP
TPGAVLMGDAFNMRHPLTGGGMTVALSDIVVLRNLLKPMRDLNDAPTL
CKYLESFYTLRKPVASTINTLAGALYKVFSASPDEARKEMRQACFDYL
SLGGLFSEGPISLLSGLNPRPLSLVLHFFAVAVFGVGRLLLPFPSPKR
VWIGARLLSGASGIILPIIKAEGIRQMFFPATVPAYYRAPPVNAF
Hypholoma sublateritium SQE
(SEQ ID NO: 68)
MSKSRSNYDVIIVGAGIAGCALAHGLSTLSRATPLRIAIVERSLAEPD
RIVGELLQPGGVMALQRLGMEGCLEGIDAVKVHGYCVVENGTSVHIPY
PGVHEGRSFHHGRFIMKLREAARAARGVELVEATVTELIPREGGKGIA
GVRVARKGKDGEEDTTEALGAALVVVADGCFSNFRAAVMGGAAVKPET
KSHFVGAILKDARLPIPNHGTVALVKGFGPVLLYQISEHDTRMLVDVK
APLPADLKVCAHILSNIVPQLPAALHLPIQRALDAERLRRMPNSFLPP
VEQGATRGAVLVGDAWNMRHPLTGGGMTVALNDVVVLRDLLGSVGDLG
DWRQVASTVNILSVALYDLFGADGELQVLRTGCFKYFERGGDCIDGPV
SLLSGIAPSPMLLAYHFFSVAFYSIYVIAVGAQNGSAKQVLAVPGALQ
YPALCVKGLRVFYTACVVFGPLLWTELRW
Hypholoma sublateritium SQE2
(SEQ ID NO: 69)
MHPTHYDVVIVGAGVAGSSLAHALATLPREKPLQIALIERSFEEPDRI
VGELLQPGGVDALKTLKMTSSVEGIDAITVTGYILVESGDMVRIPYPK
GKEGRSFHHGRFIMGLRRVALENPNVHPIEATAADLIECPCTGQVIGV
RATSKTAPAPSSIDAQQTPPAPFSVYGDLVIVADGCFSNFRNVVMGKA
ACKATTKSYFVGTILKDAVLPVAGHGTVILPQGSGPVLLYQISEHDTR
MLIDIQHPLPSDLRAHILTNILPQLPASIQGVVSDAFTKDRIRRMPNS
FLPSVQQGSPLSKKGVILLGDSWNMRHPLTGGGMTVALNDVVYLRSIF
ASIQNLDDWDEIRYALRHWHWGRKPLSSTINILSGTLYGLFEKDDDDY
RALRKGCFKYFQLGGKCIDDPVSLLSGLSPSPLLLSSHFFAVILYAIW
VVFTHPRVGSSMSANPADVKRVYDIPSADEYPQLTLKGIRMFSQACGV
FLPVLWSEIRWWAPCESS
Hypholoma sublateritium SQE3
(SEQ ID NO: 70)
MSKSRSNYDVIIVGAGIAGCALAHGLSTLSRATPLRIAIVERSLAEPD
RIVGELLQPGGVMALQRLGMEGCLEGIDAVKVHGYCVVENGTSVHIPY
PGVHEGRSFHHGRFIMKLREAARAARGVELVEATVTELIPREGGKGIA
GVRVARKGKDGEEDTTEALGAALVVVADGCFSNFRAAVMGGAAVKPET
KSHFVGAILKDARLPIPNHGTVALVKGFGPVLLYQISEHDTRMLVDVK
APLPADLKAHILSNIVPQLPAALHLPIQRALDAERLRRMPNSFLPPVE
QGATRGAVLVGDAWNMRHPLTGGGMTVALNDVVVLRDLLGSVGDLGDW
RQVRRALHRWHWDRKPLASTVNILSVALYDLFGADGEELQVLRTGCFK
YFERGGDCIDGPVSLLSGIAPSPMLLAYHFFSVAFYSIYVMFAHPQPV
AQSKAVGAQNGSAKQVLAVPGALQYPALCVKGLRVFYTACVVFGPLLW
TELRWWTAAEASRGRLLVMSLVPLLLLLGAANYGIPGMGLLGVL
Dammarenediol-II Synthases
Panax quinquefolius DDS (DDS1)
(SEQ ID NO: 3)
MAWKLKVAQGNDPYLYSTNNFVGRQYWEFQPDAGTPEEREEVEKARKD
YVNNKKLHGIHPCSDMLMRRQLIKESGIDLLSIPPVRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGSVLSYVMLRLLG
EGLAESDDGNGAVERGRKWILDHGGAAGIPSWGKTYLAVLGVYEWEGC
NPLPPEFWLFPSSFPFHPAKMWIYCRCTYMPMSYLYGKRYHGPITDLV
LSLRQEIYNIPYEQIKWNQQRHNCCKEDLYYPHTLVQDLVWDGLHYFS
EPFLKRWPFNKLRKRGLKRVVELMRYGATETRFITTGNGEKALQIMSW
WAEDPNGDEFKHHLARIPDFLWIAEDGMTVQSFGSQLWDCILATQAII
ATNMVEEYGDSLKKAHFFIKESQIKENPRGDFLKMCRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIEDSVAKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAILGLMFGGQAERDPTPLHRAAKLLINAQMD
NGDFPQQEITGVYCKNSMLHYAEYRNIFPLWALGEYRKRVWLPKHQQL
KI
Panax ginseng DDS (DDS2)
(SEQ ID NO: 4)
MAWKLKVAQGNDPYLYSTNNFVGRQYWEFQPDAGTPEEREEVEKARKD
YVNNKKLHGIHPCSDMLMRRQLIKESGIDLLSIPPLRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGSVLSYVMLRLLG
EGLAESDDGNGAVERGRKWILDHGGAAGIPSWGKTYLAVLGVYEWEGC
NPLPPEFWLFPSSFPFHPAKMWIYCRCTYMPMSYLYGKRYHGPITDLV
LSLRQEIYNIPYEQIKWNQQRHICCKEDLYYPHTLVQDLVWDGLHYFS
EPFLKRWPFNKLRKRGLKRVVELMRYGATETRFITTGNGEKALQIMSW
WAEDPNGDEFKHHLARIPDFLWIAEDGMTVQSFGSQLWDCILATQAII
ATNMVEEYGDSLKKVHFFIKESQIKENPRGDFLKMCRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIEDSVAKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAMLGLMFGGQAERDPTPLHRAAKLLINAQMD
NGDFPQQEITGVYCKNSMLHYAEYRNIFPLWALGEYRKRVWLPKHQQL
KI
DDS3
(SEQ ID NO: 5)
MAWKLKVAQGNDPYLYSTNNFVGRQYWEFQPDAGTPE
EREEVEKARKDYVNNKKLHGIHPCSDMLMRRQLIKESGIDLLSIPPVR
LDENEQVNYDAVTTAVKKALRLNRAIQAHDGHWPAENAGSLLYTPPLI
IALYISGTIDTILTKQHKKELIRFVYNHQNEDGGWGSYIEGHSTMIGS
VLSYVMLRLLGEGLAESDDGNGAVERGRKWILDHGGAAGIPSWGKTYL
AVLGVYEWEGCNPLPPEFWLFPSSFPFHPAKMWIYCRCTYMPMSYLYG
KRYHGPITDLVLSLRQEIYNIPYEQIKWNQQRHNCCKEDLYYPHTLVQ
DLVWDGLHYFSEPFLKRWPFNKLRKRGLKRVVELMRYGATETRFITTG
NGEKALQIMSWWAEDPNGDEFKHHLARIPDFLWIAEDGMTVQSFGSQL
WDCILATQAIIATNMVEEYGDSLKKAHFFIKESQIKENPRGDFLKMCR
QFTKGAWTFSDQDHGCVVSDCTAEALKCLLLLSQMPQDIVGEKPEVER
LYEAVNVLLYLQSRVSGGFAVWEPPVPKPYLEMLNPSEIFADIVVERE
HIECTASVIKGLMAFKCLHPGHRQKEIEDSVAKAIRYLERIQMPDGSW
YGFWGICFLYGTFFALSGLASAGRTYDNSEAVRKGVKFFLSTQNEEGG
WGESLESCPSEKFTPLKGNRTNLVQTSWAILGLMFGGQAERDPTPLHR
AAKLLINAQMDNGDFPQQEITGVYCKNSMLHYAEYRNIFPLWALGEYR
KRVWLPKHQQLKI
DDS4
(SEQ ID NO: 6)
MAWKLKVAQGNDPYLYSTNNFVGRQYWEFQPDAGTPEEREEVEKARKD
YVNNKKLHGIHPCSDMLMRRQLIKESGIDLLSIPPVRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGSVLSYVMLRLLG
EGLAESDDGNGAVERGRKWILDHGGAAGIPSWGKTYLAVLGVYEWEGC
NPLPPEFWLFPSSFPFHPAKMWIYCRCTYMPMSYLYGKRYHGPITDLV
LSLRQEIYNIPYEQIKWNQQRHNCCKEDLYYPHTLVQDLVWDGLHYFS
EPFLKRWPFNKLRKRGLKRVVELMRYGAEETRYITTGNGEKALQIMSW
WAEDPNGDEFKHHLARIPDFLWIAEDGMTVQSFGSQLWDCILATQAII
ATNMVEEYGDSLKKAHFFIKESQIKENPSGDFLKMCRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIEDSVAKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAILGLMFGGQAERDPTPLHRAAKLLINAQMD
NGDFPQQEITGVYCKNSMLHYAEYRNIFPLWALGEYRKRVWLPKHQQL
KI
DDS5
(SEQ ID NO: 7)
MAWKLKVAQGNDPYLYSTNNFVGRQYWEFDPDAGTPEEREEVEKARKD
YVNNKKLHGIHPCSDLLMRMQLIKESGIDLLSIPPVRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGSVLSYVMLRLLG
EGLAESDDGNGAVERGRKWILDHGGAAGIPSWGKTYLAVLGVYEWEGC
NPLPPEFWLFPSSFPFHPAKMWIYCRCTYMPMSYLYGKRYHGPITDLV
LSLRQEIYNIPYEQIKWNQQRHNCCKEDLYYPHTLVQDLVWDGLHYFS
EPFLKRWPFNKLRKRGLKRVVELMRYGATETRFITTGNGEKALQIMSW
WAEDPNGDEFKHHLARIPDFLWIAEDGMTVQSFGSQLWDCILATQAII
ATNMVEEYGDSLKKAHFFIKESQIKENPRGDFLKMCRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIEDSVAKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAILGLMFGGQAERDPTPLHRAAKLLINAQMD
NGDFPQQEITGVYCKNSMLHYAEYRNIFPLWALGEYRKRVWLPKHQQL
KI
DDS6
(SEQ ID NO: 8)
MAWKLKVAQGNDPYLYSTNNFVGRQYWEFQPDAGTPEEREEVEKARKD
YVNNKKLHGIHPCSDMLMRRQLIKESGIDLLSIPPVRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGSVLSYVMLRLLG
EGLAESDDGNGAVERGRKWILDHGGAAGIPSWGKTYLAVLGVYEWEGC
NPLPPEFWLFPSSFPFHPAKMWIYCRCTYMPMSYLYGKRYHGPITDLV
LSLRQEIYNIPYEQIKWNQQRHNCCKEDLYYPHTLVQDLVWDGLHYFS
EPFLKRWPFNKLRKRGLKRVVELMRYGATETRFITTGNGEKALQIMSW
WAEDPNGDEFKHHLARIPDFLWIAEDGMTVQSFGSQLWDCALATQAII
ATNMVEEYGDSLKKAHFFIKESQIKENPRGDFKKMYRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIEDSVAKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAILGLMFGGQAERDPTPLHRAAKLLINAQMD
NGDFPQQEITGVYCKNSMLHYAEYRNIFPLWALGEYRKRVWLPKHQQL
KI
Panax vietnamensis DDS (DDS7)
(SEQ ID NO: 9)
MAWKLKVAQGNDPYLYSTNNFVGRQYWEFLPEAGTPEEREEVEKARKD
YVNNKKLHGIHPCSDMLMRRQLIKESGIDLLSIPPVRLDENEQVNYDA
VTTAVKKALRLNRAIQAHDGHWPAENSGSLLYTPPLIIALYISGTIDT
TLTKQHKKELIRYVYNHQNEDGGWGSYIEGSSTMIGSVLSYVMLRLLG
EGSAESDDGNGAVERGRKWILDHGGAAGIPSWGKTYLAVLGVYEWEGC
NPLPPEFWLFPSFFPFHPAKMWIYCRCTYMPMSYLYGKRYHGPITDLV
LSLRQEIYNIPYEQIKWNLQRHNCCKEDLYYPHTLVQDLVWDGLHYFS
EPFLKRWPFNKLRKRGLKRVVELMRYGATETRFITTGCGEKALQIMSW
WAEDPNGDEFKHHLARVPDFLWIAEDGMTVQSFGSQLWDCILATQAII
ATNMVEEYGDSLKKAHFYIKESQIKENPRGDFLKMCRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIENSVVKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAMLGLMFGGQAERDPTPLHRAAKLLINAQMD
NGDFPQQEITGVYCKNSMLHYAEYRNIFPLWALGEYRKRVWLPKHQQL
KI
Panax quinquefolius DDS (Pq.DDS1)
(SEQ ID NO: 81)
MAWKLKVAQGNDPYLYSTNNFVGRQYWEFDPDAGTPEEREEVEKARKD
YVNNKKLHGIHPCSDLLMRMQLIKESGIDLLSIPPVRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGSVLSYVMLRLLG
EGLAESDDGNGAVERGRKWILDHGGAAGIPSWGKTYLAVLGVYEWEGC
NPLPPEFWLFPSSFPFHPAKMWIYCRCTYMPMSYLYGKRYHGPITDLV
LSLRQEIYNIPYEQIKWNQQRHNCCKEDLYYPHTLVQDLVWDGLHYFS
EPFLKRWPFNKLRKRGLKRVVELMRYGAEETRYITTGNGEKALQIMSW
WAEDPNGDEFKHHLARIPDFLWIAEDGMTVQSFGSQLWDCALATQAII
ATNMVEEYGDSLKKAHFFIKESQIKENPSGDFKKMYRQFTKGAWTFSD
QDHGCVVSDCTAEALKCLLLLSQMPQDIVGEKPEVERLYEAVNVLLYL
QSRVSGGFAVWEPPVPKPYLEMLNPSEIFADIVVEREHIECTASVIKG
LMAFKCLHPGHRQKEIEDSVAKAIRYLERNQMPDGSWYGFWGICFLYG
TFFTLSGFASAGRTYDNSEAVRKGVKFFLSTQNEEGGWGESLESCPSE
KFTPLKGNRTNLVQTSWAILGLMFGGQAERDPTPLHRAAKLLINAQMD
NGDFPQQEITGVYCKNSMLHYAEYRNIFPLWALGEYRKRVWLPKHQQL
KI
Pq. DDS2
(SEQ ID NO: 82)
MAWKLKVAQGNDPYLYSTNNFVGRQYWEFDPDAGTPEEREEVEKARKD
FVNNKKLHGIHPCSDLLMRMQLIKESGIDLLSIPPVRLDENEQVNYDA
VTTAVKKALRLNRAIQAHDGHWPAENAGSLLYTPPLIIALYISGTIDT
ILTKQHKKELIRFVYNHQNEDGGWGSYIEGHSTMIGTVLSYVMLRLLG
EGPDDGNGAVERGRKWILDHGGAAGIPSWGKTYLAVLGVYEWSGCNPL
PPEFWLFPSSFPFHPAKMWIYCRCTYMPMSYLYGKRYHGPITDLVLSL
RQEIYNIPYEQIKWNQQRHNCCKEDLYYPHTLVQDLVWDGLHYFSEPF
LKRWPFNKLRKRGLKRVVELMRYGAEETRYITTGNGEKALQIMSWWAE
DPNGDEFKHHLARIPDFLWVAEDGMTVQSFGSQLWDCALATQAIIATN
MVEEYGDSLKKAHFFIKESQIKENPSGDFKKMYRQFTKGAWTFSDQDH
GCVVSDCTAEALKCLLLLSQMPQEIVGEKPEVERLYEAVNVLLYLQSR
VSGGFAVWEPPVPKPYLEMLNPSEIFADIVVEREHIECTASVIKGLMA
FKCLHPGHRQKEIEDSVAKAIRYLERNQMPDGSWYGFWGICFLYGTFF
TLSGFASAGKTYDNSEAVRKGVKFFLSTQNEEGGWGESLESCPSEKFT
PLKGNRTNLVQTSWAILGLIFGGQAERDPTPLHRAAKLLINAQMDNGD
FPQQEITGVYCKNSMLHYAEYRNIFPLWALGEYRKRVWLPKHQQLKI
Pq. DDS3
(SEQ ID NO: 85)
MAWKLKVAQGNDPYLYSTNNFVGRQYWEFDPDAGTPEEREEVEKARKD
FVNNKKLHGIHPCSDLLMRMQLIKESGIDLLSIPPVRLDENEQVNYDA
VTTAVKKALRLNRAIQAHDGHWPSENAGSLLYTPPLIIALYISGTIDT
ILTKEHKKELIRFVYNHQNEDGGWGSYIEGHSTMIGTVLSYVMLRLLG
EGPDDGNGAVERGRKWILDHGGAAGIPSWGKTYLAVLGVYEWSGCNPL
PPEFWLFPSSFPFHPGKMWIYCRCTYMPMSYLYGKRYHGPITDLVLSL
RQEIYNIPYEQIKWNQQRHNCCKEDLYYPHTLVQDLVWDGLHYFSEPF
LKRWPFNKLRKRGLKRVVELMRYGAEETRYITTGNGEKALQIMAWWAE
DPNGDEFKHHLARIPDFLWVAEDGMTVQSFGSQLWDCALATQAIIATN
MVEEYGDSLKKAHFFIKESQIKENPSGDFKKMYRQFTKGAWTFSDQDH
GCVVSDCTAEALKCLLLLSQMPQEIVGEKPEVERLYEAVNVLLYLQSR
VSGGFAVWEPPVPKPYLEMENPSEIFADIVVEREHIECTASVIKALMA
FKCLHPGHRQKEIEDSVAKAIRYLERNQMPDGSWYGFWGICFLYGTFF
TLSGFASAGKTYDNSEAVRKGVKFLLSTQNEEGGWGESLESCPSEKFT
PLKGNRTNLVQTSWAILGLIFGGQAERDPTPLHRAAKLLINAQMDNGD
FPQQEITGVYCKNSMLHYAEYRNIFPLWALGEYRKRVWLPKHQQLKI
Protopanaxadiol Synthase
Panax ginseng PPDS (PPDS1)
(SEQ ID NO: 10)
MAQDLRLILIIVGAIAIIALLVHGFWSYTKRIPQKENDSKAPLPPGQT
GWPLIGETLNYLSCVKSGVSENFVKYRKEKYSPKVFRTSLLGEPMAIL
CGPEGNKFLYSTEKKLVQVWFPSSVEKMFPRSHGESNADNFSKVRGKM
MFLLKVDGMKKYVGLMDRVMKQFLETDWNRQQQINVHNTVKKYTVTMS
CRVFMSIDDEEQVTRLGSSIQNIEAGLLAVPINIPGTAMNRAIKTVKL
LTREVEAVIKQRKVDLLENKQASQPQDLLSHLLLTANQDGQFLSESDI
ASHLIGLMQGGYTTLNGTITFVLNYLAEFPDVYNQVLKEQVEIANSKH
PKELLNWEDLRKMKYSWNVAQEVLRIIPPGVGTFREAITDFTYAGYLI
PKGWKMHLIPHDTHKNPTYFPSPEKFDPTRFEGNGPAPYTFTPFGGGP
RMCPGIEYARLVILIFMHNVVTNFRWEKLIPNEKILTDPIPRFAHGLP
IHLHPHN
Panax notoginseng PPDS (PPDS2)
(SEQ ID NO: 11)
MAQDLRLILIIVGAIAIIALLVHGFAYFSYTKRIPQKENDSKAPLPPG
QTGWPLIGETLNYLSCVKSGFSENFVKYRKEKYSPKVFRTSLLGEPMA
ILCGPEGNKFLYSTEKKLVQTWFPSSVEKMFPRSHGESNADNFSKVRG
KMMFLLKVDGLKKYVGLMDRVMKQFLETDWNRQQQINVHNTVKKYTVT
MSCRVFMSIDDEEQVRRLGSSIQNIEAGLLAVPINIPGTAMNRAIKTV
KLLSREVEAVIKQRKVDLLENKQASQPQDLLSHLLLTANQDGQFLSES
DIASHLIGLMQGGYTTLNGTITFVINYLAEFPDVYNQVLKEQVEIANS
KHPKELLNWEDLRKMKYSWNVAQEVLRIIPPGVGTFREAITDFTYAGY
LIPKGWKMHLIPHDTHKNPTYFPNPEKFDPTRFEGNGPAPYTFTPFGG
GPRMCPGIEYARLVILIFIHNVVTNFRWEKLIPSEKILTDPIPRFAHG
LPIHLHPHN
Panax notoginseng PPDS (PPDS3)
(SEQ ID NO: 12)
MAQDLRLILIIVGAIAII
ALLVHGFMAAAMVLFFSLSLLLLPLPLLLFAYFSYTKRIPQKENDSKA
PLPPGQTGWPLIGETLNYLSCVKSGFSENFVKYRKEKYSPKVFRTSLL
GEPMAILCGPEGNKFLYSTEKKLVQTWFPSSVEKMFPRSHGESNADNF
SKVRGKMMFLLKVDGLKKYVGLMDRVMKQFLETDWNRQQQINVHNTVK
KYTVTMSCRVFMSIDDEEQVRRLGSSIQNIEAGLLAVPINIPGTAMNR
AIKTVKLLSREVEAVIKQRKVDLLENKQASQPQDLLSHLLLTANQDGQ
FLSESDIASHLIGLMQGGYTTLNGTITFVINYLAEFPDVYNQVLKEQV
EIANSKQPKELLNWEDLRKMKYSWNVAQEVLRIIPPGVGTFREAITDF
TYAGYLIPKGWKMHLIPHDTHKNPTYFPNPEKFDPTRFEGNGPAPYTF
TPFGGGPRMCPGIEYARLVILIFIHNVVTNFRWEKLIPSEKILTDPIP
RFAHGLPIHLHPHN
Kalopanax septemlobus PPDS (PPDS4)
(SEQ ID NO: 13)
MAQDLRLILIIVGAIAIIALLVHGFAYFSYQLFITKHQGNDSKTPRLP
PGRTGWPLIGESLNYISTIKSGLLENFVTYRMGKYSPKVFRTSIFGET
MAVLCGPEGNKLIFSNERKLVRVWFPSSVDKIFPRSHGETNAENFFKV
RKMMFVLKVDALKKYVGLMDTAMKQFLRTDWNHRHQQINVYETVKKYT
VMMACRVFMSIDDAEQLGKISNLIQHIEAGLFAVPINLPGTAMNRAIK
TVELLSKDLEAVVKQRKVDLLNNKASPTQDLLSHLLLTANDDGRFLSE
SDIASHLLGLMQGGYSTLNVTITFIMNYLAELPDVYNQVLKEQVEIAN
SKSPKELLNWEDLRKMKYSWNVAQEVLRIRSPGVGTFREVIADFTYAG
YLIPKGWKIPLIPQSTFKNPAYYPNPEKFDPTRFEGNGPAPYSYTPFG
GGPRMCPGVEYARLAILIFMHNVVTNFKWEKLIPNEKIFTYPAPKFAH
GLPIQLHPHNL
Eleutherococcus senticosus PPDS (PPDS5)
(SEQ ID NO: 14)
MAQDLRLILIIVGAIAIIALLVHGFAYFSHQIFITKHRNTDSKIPLPP
GPTGWPLIGESLNYLSTVKSGLLENFVTYRKEKYSTKVFRTSLFGESV
AILCGAEGNKFLFSNERKLVRVWFPRSVEKIFAQSHAESNAESFYKIR
KMMFILKADALKKYVGLMDTIMKQFLQTHWNHHLQTQINVHNTVMNYS
LMLSCRVFMSIDDAEQVRKIGNSIHHIEAGLFAVPINLPGTAMNRAIK
TVKLLSKEFEAVVKQRKADLLENKQAPPTQDLLSHLLLTPNEDGRFMS
ESDIARQLLGLVQGAYSTLNVVIAFIINYLAELPDVYDQVLKEQVEIA
KSKNPKELLNWEDLSKMKYSWNVVQEVLRIRSPAIGVFREAINDFTYA
GYLIPKGWKLHLIPVATHKNPTYFPNPEKFDPTRFEGSGPAPYTFTPF
GGGPRMCPGVEYARLAILIFMHHAVTNFRWEKLIPNEQIFTFPVLSFA
NGLPIHLHPHNP
Camellia sinensis PPDS (PPDS6)
(SEQ ID NO: 15)
MAQDLRLILIIVGAIAIIALLVHGFSYLISGHTPRRANENISSENFPL
PPGRTGWPLIGESLDYFLKLRNCIPEKFVADRRDRYSTKVFKTSLLGE
PMAIFCGVEGNKFLFSSETKLVQLWWPKAISKIFPKSSADYMKEDSTK
VRKILQPFLKADALQKHVGVMDMLMKQHLDMDWNCREVKVSPAVTKYT
FMLACRLFLSIEDLERVEELGKSFGYITAGIVSMAINVPGTAFNRAIK
ASKIMRRELEAMIQQRKIDLTENRSLAAQDLLSHMLLANDENDRFMTE
FDIASHIVGLLHAATHTLNVALTFIVMYLAELPDVYNEVLREQMGIAE
SKEPEDLLNWKDIKKMKYSWNVANEVLRLRPPSFGTFREAITDFTYAG
YMIPKGWKLHLIAQTTHKNPEYFPNPETFDPSRFEGNGPPPFTFVPFG
GGPRMCPGNEYARLVMLVFMHNMVTKFRWKKVIPNEKVVIDPLPRPTQ
GLPVHLHPHKP
Panax quinquefolius PPDS (PPDS7)
(SEQ ID NO: 16)
MAQDLRLILIIVGAIAIIALLVHGFAYFSYTKRIPQKENDSKAPLPPG
QTGWPLIGETLNYLSCVKSGVSENFVKYRKEKYSPKVFRTSLLGEPMA
ILCGPEGNKFLYSTEKKLVQVWFPSSVEKMFPRSHGESNADNFSKVRC
KMMFLLKVDGMKKYVGLMDRVMKQFLESDWNRQQQINVHNTVKKYTVT
MSCRVFMSIDDEEQVTRLGSSIQNIEAGLLAVPINIPGTAMNRAIKTV
KLLTREVEAVIKQRKVDLLENKQASQPQDLLSHLLLTANQDGQFLSES
DIASHLIGLMQGGYTTLNGTITFVLNYLAEFPDVYNQVLKEQVEIANS
KHPKELLNWEDLRKMKYSWNVAQEVLRIIPPGVGTFREAITDFTYAGY
LIPKGWKMHLIPHDTHKNPTYFPSPEKFDPTRFEGNGPAPYTFTPFGG
GPRMCPGIEYARLVILIFMHNVVTNFRWEKLIPNEKILTDPIPRFAHG
LPIHLHPHN
Pg. PPDS1
(SEQ ID NO: 83)
MAQDLRLILIIVGAIAIIALLVHGFWSYTKRIPQKENDSKAPLPPGQT
GWPLIGETLEYLSCVKSGVPENFVKYRKEKYSPKVFRTSLLGEPMAIL
CGPEGNKFLYSNEKKLVQVWFPSSVEKMFPRSHGESNAENFSKVRGKM
MFLLKPDGMKKYVGLMDRVMKQHLETDWNRQQQINVHNTVKKYTVTMS
CRVFMSIDDEEQVTRLGSSFQNIEAGLLAVPINIPGTAMNRAIKTVKL
LTKEVEAVIKQRKVDLLENKQASQPQDLLSHLLLTANEDGQFMSESDI
ASHIIGLMQGGYTTLNGTITFVLNYLAEFPDVYNQVLKEQMEIANSKH
PGELLNWEDLQKMKYSWNVAQEVLRIIPPGVGTFREAITDFTYAGYLI
PKGWKLHLIPHDTHKNPTYFPSPEKFDPTRFEGNGPAPYTFTPFGGGP
RMCPGIEYARLVILIFMHNVVTNFRWEKLIPNEKILTDPIPRFAHGLP
IRLHPHN
Protopanaxatriol Synthase
Panax ginseng PPTS (PPTS1)
(SEQ ID NO: 17)
MAQDLRLILIIVGAIAIIALLVHGFWNFKPSSQNKLPPGKTGWPIIGE
TLEFISCGQKGNPEKFVTQRMNKYSPDVFTTSLAGEKMVVFCGASGNK
FIFSNENKLVVSWWPPAISKILTATIPSVEKSKALRSLIVEFLKPEAL
HKFISVMDRTTRQHFEDKWNGSTEVKAFAMSESLTFELACWLLFSIND
PVQVQKLSHLFEKVKAGLLSLPLNFPGTAFNRGIKAANLIRKELSVVI
KQRRSDKLQTRKDLLSHVMLSNGEGEKFFSEMDIADVVLNLLIASHDT
TSSAMGSVVYFLADHPHIYAKVLTEQMEIAKSKGAEELLSWEDIKRMK
YSRNVINEAMRLVPPSQGGFKVVTSKFSYANFIIPKGWKIFWSVYSTH
KDPKYFKNPEEFDPSRFEGDGPMPFTFIPFGGGPRMCPGSEFARLEVL
IFMHHLVTNFKWEKVFPNEKIIYTPFPFPENGLPIRLSPCTL
Panax quinquefolius PPTS (PPTS2)
(SEQ ID NO: 18)
MAQDLRLILIIVGAIAIIALLVHGFWNFKPSSQNKLPPGKTGWPIIGE
TLEFISCGQKGNPEKFVTQRMKKYSPDVFTTSLAGEKMVVFCGASGNK
FIFSNENKLVVSWWPPAISKILTATIPSVEKSKALRSLIVEFLKPEAL
HKFISVMDRTTRQHFEDKWNGSTEVKAFAMSESLTFELACWLLFSIND
PVQVQKLSHLFEKVKAGLLSLPLNFPGTAFNRGIKAANLIRKELSVMI
KQRRSDKLQTRKDLLSHVMLSNGEGEKFFSEMDIADVVLNLLIASHDT
TSSAMGSVVYFLADHPHIYAKVLTEQMEIAKSKGAGELLSWEDIKRMK
YSRNVINEAMRLVPPSQGGFKVVTSKFSYANFIIPKGWKIFWSVYSTH
KDPKYFKNPEEFDPSRFEGDGPMPFTFIPFGGGPRMCPGSEFARLEVL
IFMHHLVTNFRWDKVFPNEKIIYTPFPSTENSRTIRLSPCTL
Panax notoginseng PPTS (PPTS3)
(SEQ ID NO: 19)
MAQDLRLILIIVGAIAIIALLVHGFFWNFKPSSQNKLPPGKTGWPIIG
ETLEFISCGQKGNPEKFVTQRMKKYSPDVFTTSLAGEKMVVFCGASGN
KFIFSNENKLVVSWWPPAISKILTATIPSVEKSKALRSLIVEFLKPEA
LHKFISVMDRTTRQHFEAKWNGSTEVKAFAMSETLTFELACWLLFSIS
DPVQVQKLSHLFEKVKAGLLSLPLNFPGTAFNRGIKAANLIRKELSVV
IKQRRSDKSETRKDLLSHVMISNGEGEKFFSEMDIADVVLNILIASHD
TTSSAMGSVVYFLADHPHIYAKVLAEQMEIAKSKGAGELLSWEDIKRM
KYSRNVINEAMRLVPPSQGGFKVVTSKFSYANFIIPKGWKIFWSVYST
HKDPKYFKNPEEFDPSRFEGDGPMPFTFIPFGGGPRMCPGSEFARLEV
LIFMHHLVTNFRWEKVFPNEKIIYTPFPFPENGLPIRLSPCTL
Eleutherococcus senticosus PPTS (PPTS4)
(SEQ ID NO: 20)
MAQDLRLILIIVGAIAIIALLVHGFLRPSSQNKLPPGKTGWPIIGETL
EYLSWGQKGCPEKFITQRMNKYSPHVFTTSLAGEKMAIFCGASGNKFM
FSNENKLVVSWWPPAISKIVNSTKPSVEKSKAVRNLIVEFLKPEALHK
FIPVMDRTTRLHFEAEWGGTTEVKAFALSEMLTFELACRLLCSIDDPV
HVKTLSCLFAKVKAGLMSLPIDFPGTAFNSGIKAANLIRKDLSVLIEQ
RRSDKLQIRGDLLSHILISNGEDEKILSEMDIADVVLGLLIASHDTTS
SVMASVVYFLTDHPGIYAKVLTEQMEIAKSKRAGDLLTWENIQRMKYS
RNVINEVMRLVPPSQGGFKEVISEFSYADFIIPKGWKIFWSVHSTHKD
PKYFKNPEEFDPPRFEGDGPMPFTFIPFGGGPRMCPGNEFARMEVLIF
MHHLVMNFRWEKVFPNEKIIYTSFPFPEKGLPIRLSPCTL
Panax notoginseng PPTS (PPTS5)
(SEQ ID NO: 21)
MAQDLRLILIIVGAIAIIALLVHGFFWNFKPSSQNKLPPGKTGWPIIG
ETLEFISWGQKGNPEKFVTQRMKKYSPDVFTTSLAGEKMVVFCGASGN
KFIFSNENKLVVSWWPPAISKILTATIPSVEKSKALRSLIVEFLKPEA
LHKFISVMDRTTRQHFEAKWNGSTEVKAFAMSETLTFELACWLLFSIN
DPVQVQKLSHLFEKVKAGLLSLPLNFPGTAFNRGIKAANLIRKELSVV
IKQRRSDKSETRKDLLSHVMISNGEGEKFFSEMDIADVVLNILIASHD
TTSSAMGSVVYFLADHPHIYAKVLTEQMEIAKSKGAGELLSWEDIKRM
KYSRNVINEAMRLVPPSQGGFKVVTSKFSYANFIIPKGWKIFWSVYST
HKDPKYFKNPEEFDPSRFEGDGPMPFTFIPFGGGPRMCPGSEFARLEV
LIFMHHLVTNFRWEKVFPNEKIIYTPFPFPENGLPIRLSPCTL
Pg. PPTS2
(SEQ ID NO: 84)
MAQDLRLILIIVGAIAIIALLVHGFWNFKPSSQNKLPPGKTGWPIIGE
TLEFISCGQKGNPEKFVTQRMNKYSPDVFTTSLAGEKMVVFCGASGNK
FIFSNENKLVVSWWPPAISKILTATIPSVEKSKALRSLIVEFLKPEAL
HKFISVMDRTTRQHFEDKWNGKTEVKAFAMSESLTFELACWLLFSIND
PVQVQKLSHLFEKVKAGLLSLPLNFPGTAFNRGIKAANLIRKELSVII
KQRRSDKLQPRQDLLSHVMLSNGEGEKFFSEMDIADVVLNLLIASHDT
TSSAMTSVVYFLADHPHIYAKVLTEQMEIAKSKGPEELLSWEDIKRMK
YSRNVINEAMRLVPPSQGGFKVVTSDFSYANFTIPKGWKIFWSVYSTH
KDPKYFKNPEEFDPSRFEGDGPMPFTFVPFGGGPRMCPGSEFARLEVL
IFMHHLVTNFKWEKVFPNEKIIYTPFPFPENGLPIRLSPHTL
Cytochrome P450 Reductases
Camptotheca acuminate Cytochrome
P450 Reductase (CPR1)
(SEQ ID NO: 22)
MAQSSSVKVSTFDLMSAILRGRSMDQTNVSFESGESPALAMLIENREL
VMILTTSVAVLIGCFVVLLWRRSSGKSGKVTEPPKPLMVKTEPEPEVD
DGKKKVSIFYGTQTGTAEGFAKALAEEAKVRYEKASFKVIDLDDYAAD
DEEYEEKLKKETLTFFFLATYGDGEPTDNAARFYKWFMEGKERGDWLK
NLHYGVFGLGNRQYEHFNRIAKVVDDTIAEQGGKRLIPVGLGDDDQCI
EDDFAAWRELLWPELDQLLQDEDGTTVATPYTAAVLEYRVVFHDSPDA
SLLDKSFSKSNGHAVHDAQHPCRANVAVRRELHTPASDRSCTHLEFDI
SGTGLVYETGDHVGVYCENLIEVVEEAEMLLGLSPDTFFSIHTDKEDG
TPLSGSSLPPPFPPCTLRRALTQYADLLSSPKKSSLLALAAHCSDPSE
ADRLRHLASPSGKDEYAQWVVASQRSLLEVMAEFPSAKPPIGAFFAGV
APRLQPRYYSISSSPRMAPSRIHVTCALVFEKTPVGRIHKGVCSTWMK
NAVPLDESRDCSWAPIFVRQSNFKLPADTKVPVLMIGPGTGLAPFRGF
LQERLALKEAGAELGPAILFFGCRNRQMDYIYEDELNNFVETGALSEL
IVAFSREGPKKEYVQHKMMEKASDIWNMISQEGYIYVCGDAKGMARDV
HRTLHTIVQEQGSLDSSKTESMVKNLQMNGRYLRDVW
Stevia rebaudiana (SrCPR1)
(SEQ ID NO: 71)
MAQSDSVKVSPFDLVSAAMNGKAMEKLNASESEDPTTLPALKMLVENR
ELLTLFTTSFAVLIGCLVFLMWRRSSSKKLVQDPVPQVIVVKKKEKES
EVDDGKKKVSIFYGTQTGTAEGFAKALVEEAKVRYEKTSFKVIDLDDY
AADDDEYEEKLKKESLAFFFLATYGDGEPTDNAANFYKWFTEGDDKGE
WLKKLQYGVFGLGNRQYEHFNKIAIVVDDKLTEMGAKRLVPVGLGDDD
QCIEDDFTAWKELVWPELDQLLRDEDDTSVTTPYTAAVLEYRVVYHDK
PADSYAEDQTHINGHVVHDAQHPSRSNVAFKKELHTSQSDRSCTHLEF
DISHTGLSYETGDHVGVYSENLSEVVDEALKLLGLSPDTYFSVHADKE
DGTPIGGASLPPPFPPCTLRDALTRYADVLSSPKKVALLALAAHASDP
SEADRLKFLASPAGKDEYAQWIVANQRSLLEVMQSFPSAKPPLGVFFA
AVAPRLQPRYYSISSSPKMSPNRIHVTCALVYETTPAGRIHRGLCSTW
MKNAVPLTESPDCSQASIFVRTSNFRLPVDPKVPVIMIGPGTGLAPFR
GFLQERLALKESGTELGSSIFFFGCRNRKVDFIYEDELNNFVETGALS
ELIVAFSREGTAKEYVQHKMSQKASDIWKLLSEGAYLYVCGDAKGMAK
DVHRTLHTIVQEQGSLDSSKAELYVKNLQMSGRYLRDVW
Arabidopsis thaliana CPR1 (AtCPR1)
(SEQ ID NO: 72)
MATSALYASDLFKQLKSIMGTDSLSDDVVLVIATTSLALVAGFVVLLW
KKTTADRSGELKPLMIPKSLMAKDEDDDLDLGSGKTRVSIFFGTQTGT
AEGFAKALSEEIKARYEKAAVKVIDLDDYAADDDQYEEKLKKETLAFF
CVATYGDGEPTDNAARFYKWFTEENERDIKLQQLAYGVFALGNRQYEH
ENKIGIVLDEELCKKGAKRLIEVGLGDDDQSIEDDFNAWKESLWSELD
KLLKDEDDKSVATPYTAVIPEYRVVTHDPRFTTQKSMESNVANGNTTI
DIHHPCRVDVAVQKELHTHESDRSCIHLEFDISRTGITYETGDHVGVY
AENHVEIVEEAGKLLGHSLDLVFSIHADKEDGSPLESAVPPPFPGPCT
LGTGLARYADLLNPPRKSALVALAAYATEPSEAEKLKHLTSPDGKDEY
SQWIVASQRSLLEVMAAFPSAKPPLGVFFAAIAPRLQPRYYSISSSPR
LAPSRVHVTSALVYGPTPTGRIHKGVCSTWMKNAVPAEKSHECSGAPI
FIRASNFKLPSNPSTPIVMVGPGTGLAPFRGFLQERMALKEDGEELGS
SLLFFGCRNRQMDFIYEDELNNFVDQGVISELIMAFSREGAQKEYVQH
KMMEKAAQVWDLIKEEGYLYVCGDAKGMARDVHRTLHTIVQEQEGVSS
SEAEAIVKKLQTEGRYLRDVW
Arabidopsis thaliana CPR2 (AtCPR2)
(SEQ ID NO: 73)
MASSSSSSSTSMIDLMAAIIKGEPVIVSDPANASAYESVAAELSSMLI
ENRQFAMIVTTSIAVLIGCIVMLVWRRSGSGNSKRVEPLKPLVIKPRE
EEIDDGRKKVTIFFGTQTGTAEGFAKALGEEAKARYEKTRFKIVDLDD
YAADDDEYEEKLKKEDVAFFFLATYGDGEPTDNAARFYKWFTEGNDRG
EWLKNLKYGVFGLGNRQYEHFNKVAKVVDDILVEQGAQRLVQVGLGDD
DQCIEDDFTAWREALWPELDTILREEGDTAVATPYTAAVLEYRVSIHD
SEDAKFNDINMANGNGYTVFDAQHPYKANVAVKRELHTPESDRSCIHL
EFDIAGSGLTYETGDHVGVLCDNLSETVDEALRLLDMSPDTYFSLHAE
KEDGTPISSSLPPPFPPCNLRTALTRYACLLSSPKKSALVALAAHASD
PTEAERLKHLASPAGKDEYSKWVVESQRSLLEVMAEFPSAKPPLGVFF
AGVAPRLQPRFYSISSSPKIAETRIHVTCALVYEKMPTGRIHKGVCST
WMKNAVPYEKSENCSSAPIFVRQSNFKLPSDSKVPIIMIGPGTGLAPF
RGFLQERLALVESGVELGPSVLFFGCRNRRMDFIYEEELQRFVESGAL
AELSVAFSREGPTKEYVQHKMMDKASDIWNMISQGAYLYVCGDAKGMA
RDVHRSLHTIAQEQGSMDSTKAEGFVKNLQTSGRYLRDVW
Arabidopsis thaliana (AtCPR3)
(SEQ ID NO: 74)
MASSSSSSSTSMIDLMAAIIKGEPVIVSDPANASAYESVAAELSSMLI
ENRQFAMIVTTSIAVLIGCIVMLVWRRSGSGNSKRVEPLKPLVIKPRE
EEIDDGRKKVTIFFGTQTGTAEGFAKALGEEAKARYEKTRFKIVDLDD
YAADDDEYEEKLKKEDVAFFFLATYGDGEPTDNAARFYKWFTEGNDRG
EWLKNLKYGVFGLGNRQYEHFNKVAKVVDDILVEQGAQRLVQVGLGDD
DQCIEDDFTAWREALWPELDTILREEGDTAVATPYTAAVLEYRVSIHD
SEDAKFNDITLANGNGYTVFDAQHPYKANVAVKRELHTPESDRSCIHL
EFDIAGSGLTMKLGDHVGVLCDNLSETVDEALRLLDMSPDTYFSLHAE
KEDGTPISSSLPPPFPPCNLRTALTRYACLLSSPKKSALVALAAHASD
PTEAERLKHLASPAGKDEYSKWVVESQRSLLEVMAEFPSAKPPLGVFF
AGVAPRLQPRFYSISSSPKIAETRIHVTCALVYEKMPTGRIHKGVCST
WMKNAVPYEKSEKLFLGRPIFVRQSNFKLPSDSKVPIIMIGPGTGLAP
FRGFLQERLALVESGVELGPSVLFFGCRNRRMDFIYEEELQRFVESGA
LAELSVAFSREGPTKEYVQHKMMDKASDIWNMISQGAYLYVCGDAKGM
ARDVHRSLHTIAQEQGSMDSTKAEGFVKNLQTSGRYLRDVW
Stevia rebaudiana CPR2 (SrCPR2)
(SEQ ID NO: 75)
MAQSESVEASTIDLMTAVLKDTVIDTANASDNGDSKMPPALAMMFEIR
DLLLILTTSVAVLVGCFVVLVWKRSSGKKSGKELEPPKIVVPKRRLEQ
EVDDGKKKVTIFFGTQTGTAEGFAKALFEEAKARYEKAAFKVIDLDDY
AADLDEYAEKLKKETYAFFFLATYGDGEPTDNAAKFYKWFTEGDEKGV
WLQKLQYGVFGLGNRQYEHFNKIGIVVDDGLTEQGAKRIVPVGLGDDD
QSIEDDFSAWKELVWPELDLLLRDEDDKAAATPYTAAIPEYRVVFHDK
PDAFSDDHTQTNGHAVHDAQHPCRSNVAVKKELHTPESDRSCTHLEFD
ISHTGLSYETGDHVGVYCENLIEVVEEAGKLLGLSTDTYFSLHIDNED
GSPLGGPSLQPPFPPCTLRKALTNYADLLSSPKKSTLLALAAHASDPT
EADRLRFLASREGKDEYAEWVVANQRSLLEVMEAFPSARPPLGVFFAA
VAPRLQPRYYSISSSPKMEPNRIHVTCALVYEKTPAGRIHKGICSTWM
KNAVPLTESQDCSWAPIFVRTSNFRLPIDPKVPVIMIGPGTGLAPFRG
FLQERLALKESGTELGSSILFFGCRNRKVDYIYENELNNFVENGALSE
LDVAFSRDGPTKEYVQHKMTQKASEIWNMLSEGAYLYVCGDAKGMAKD
VHRTLHTIVQEQGSLDSSKAELYVKNLQMSGRYLRDVW
Stevia rebaudiana CPR3 (SrCPR3)
(SEQ ID NO: 76)
MAQSNSVKISPLDLVTALFSGKVLDTSNASESGESAMLPTIAMIMENR
ELLMILTTSVAVLIGCVVVLVWRRSSTKKSALEPPVIVVPKRVQEEEV
DDGKKKVTVFFGTQTGTAEGFAKALVEEAKARYEKAVFKVIDLDDYAA
DDDEYEEKLKKESLAFFFLATYGDGEPTDNAARFYKWFTEGDAKGEWL
NKLQYGVFGLGNRQYEHFNKIAKVVDDGLVEQGAKRLVPVGLGDDDQC
IEDDFTAWKELVWPELDQLLRDEDDTTVATPYTAAVAEYRVVFHEKPD
ALSEDYSYTNGHAVHDAQHPCRSNVAVKKELHSPESDRSCTHLEFDIS
NTGLSYETGDHVGVYCENLSEVVNDAERLVGLPPDTYFSIHTDSEDGS
PLGGASLPPPFPPCTLRKALTCYADVLSSPKKSALLALAAHATDPSEA
DRLKFLASPAGKDEYSQWIVASQRSLLEVMEAFPSAKPSLGVFFASVA
PRLQPRYYSISSSPKMAPDRIHVTCALVYEKTPAGRIHKGVCSTWMKN
AVPMTESQDCSWAPIYVRTSNFRLPSDPKVPVIMIGPGTGLAPFRGFL
QERLALKEAGTDLGLSILFFGCRNRKVDFIYENELNNFVETGALSELI
VAFSREGPTKEYVQHKMSEKASDIWNLLSEGAYLYVCGDAKGMAKDVH
RTLHTIVQEQGSLDSSKAELYVKNLQMSGRYLRDVW
Artemisia annua CPR
(AaCPR)
(SEQ ID NO: 77)
MAQSTTSVKLSPFDLMTALLNGKVSFDTSNTSDTNIPLAVFMENRELL
MILTTSVAVLIGCVVVLVWRRSSSAAKKAAESPVIVVPKKVTEDEVDD
GRKKVTVFFGTQTGTAEGFAKALVEEAKARYEKAVFKVIDLDDYAAED
DEYEEKLKKESLAFFFLATYGDGEPTDNAARFYKWFTEGEEKGEWLDK
LQYAVFGLGNRQYEHFNKIAKVVDEKLVEQGAKRLVPVGMGDDDQCIE
DDFTAWKELVWPELDQLLRDEDDTSVATPYTAAVAEYRVVFHDKPETY
DQDQLTNGHAVHDAQHPCRSNVAVKKELHSPLSDRSCTHLEFDISNTG
LSYETGDHVGVYVENLSEVVDEAEKLIGLPPHTYFSVHADNEDGTPLG
GASLPPPFPPCTLRKALASYADVLSSPKKSALLALAAHATDSTEADRL
KFLASPAGKDEYAQWIVASHRSLLEVMEAFPSAKPPLGVFFASVAPRL
QPRYYSISSSPRFAPNRIHVTCALVYEQTPSGRVHKGVCSTWMKNAVP
MTESQDCSWAPIYVRTSNFRLPSDPKVPVIMIGPGTGLAPFRGFLQER
LAQKEAGTELGTAILFFGCRNRKVDFIYEDELNNFVETGALSELVTAF
SREGATKEYVQHKMTQKASDIWNLLSEGAYLYVCGDAKGMAKDVHRTL
HTIVQEQGSLDSSKAELYVKNLQMAGRYLRDVW
CPR (PgCPR)
(SEQ ID NO: 78)
MAQSSSGSMSPFDFMTAIIKGKMEPSNASLGAAGEVTAMILDNRELVM
ILTTSIAVLIGCVVVFIWRRSSSQTPTAVQPLKPLLAKETESEVDDGK
QKVTIFFGTQTGTAEGFAKALADEAKARYDKVTFKVVDLDDYAADDEE
YEEKLKKETLAFFFLATYGDGEPTDNAARFYKWFLEGKERGEWLQNLK
FGVFGLGNRQYEHFNKIAIVVDEILAEQGGKRLISVGLGDDDQCIEDD
FTAWRESLWPELDQLLRDEDDTTVSTPYTAAVLEYRVVFHDPADAPTL
EKSYSNANGHSVVDAQHPLRANVAVRRELHTPASDRSCTHLEFDISGT
GIAYETGDHVGVYCENLAETVEEALELLGLSPDTYFSVHADKEDGTPL
SGSSLPPPFPPCTLRTALTLHADLLSSPKKSALLALAAHASDPTEADR
LRHLASPAGKDEYAQWIVASQRSLLEVMAEFPSAKPPLGVFFASVAPR
LQPRYYSISSSPRIAPSRIHVTCALVYEKTPTGRVHKGVCSTWMKNSV
PSEKSDECSWAPIFVRQSNFKLPADAKVPIIMIGPGTGLAPFRGFLQE
RLALKEAGTELGPSILFFGCRNSKMDYIYEDELDNFVQNGALSELVLA
FSREGPTKEYVQHKMMEKASDIWNLISQGAYLYVCGDAKGMARDVHRT
LHTIAQEQGSLDSSKAESMVKNLQMSGRYLRDVW
Camptotheca acuminate CaCPR
(SEQ ID NO: 79)
MAQSSSVKVSTFDLMSAILRGRSMDQTNVSFESGESPALAMLIENREL
VMILTTSVAVLIGCFVVLLWRRSSGKSGKVTEPPKPLMVKTEPEPEVD
DGKKKVSIFYGTQTGTAEGFAKALAEEAKVRYEKASFKVIDLDDYAAD
DEEYEEKLKKETLTFFFLATYGDGEPTDNAARFYKWFMEGKERGDWLK
NLHYGVFGLGNRQYEHFNRIAKVVDDTIAEQGGKRLIPVGLGDDDQCI
EDDFAAWRELLWPELDQLLQDEDGTTVATPYTAAVLEYRVVFHDSPDA
SLLDKSFSKSNGHAVHDAQHPCRANVAVRRELHTPASDRSCTHLEFDI
SGTGLVYETGDHVGVYCENLIEVVEEAEMLLGLSPDTFFSIHTDKEDG
TPLSGSSLPPPFPPCTLRRALTQYADLLSSPKKSSLLALAAHCSDPSE
ADRLRHLASPSGKDEYAQWVVASQRSLLEVMAEFPSAKPPIGAFFAGV
APRLQPRYYSISSSPRMAPSRIHVTCALVFEKTPVGRIHKGVCSTWMK
NAVPLDESRDCSWAPIFVRQSNFKLPADTKVPVLMIGPGTGLAPFRGF
LQERLALKEAGAELGPAILFFGCRNRQMDYIYEDELNNFVETGALSEL
IVAFSREGPKKEYVQHKMMEKASDIWNMISQEGYIYVCGDAKGMARDV
HRTLHTIVQEQGSLDSSKTESMVKNLQMNGRYLRDVW

TABLE 1
Pq.DDS1 Derivatives

| Pq.DDS1 derivatives | Fold improvement |
|---|---|
| L195Del3 | 1.70 |
| Y49F | 1.69 |
| M695I | 1.63 |
| S181T | 1.54 |
| S198P | 1.53 |
| R637K | 1.49 |
| E238S | 1.49 |
| T268V | 1.48 |
| G697A | 1.47 |
| G208A | 1.39 |
| I407V | 1.33 |
| D507E | 1.30 |
| F652L | 1.29 |
| D392P | 1.27 |
| V515P | 1.26 |
| A100T | 1.23 |
| I155L | 1.23 |
| G576A | 1.22 |
| V328L | 1.21 |
| G352A | 1.18 |
| N93T | 1.11 |

TABLE 2
Pq.DDS2 Derivatives

| Pq.DDS2 derivatives | Fold improvement |
|---|---|
| F649L | 1.43 |
| L548F | 1.42 |
| Q149E A120S | 1.37 |
| I155L | 1.33 |
| G573A | 1.31 |
| F244I | 1.31 |
| S380A | 1.29 |
| V325I | 1.28 |
| E40A | 1.28 |
| A256G | 1.27 |
| G694A | 1.26 |
| C262S | 1.26 |
| F253V | 1.26 |
| V325L | 1.25 |
| V593I | 1.24 |
| T147S | 1.23 |
| F244V | 1.23 |
| M258L | 1.23 |
| G349A | 1.22 |
| C262T | 1.21 |
| S551T | 1.20 |
| C579K | 1.20 |
| I688M | 1.20 |
| F251L | 1.19 |
| G372I | 1.18 |
| L78- | 1.17 |
| I111L | 1.17 |
| A383V | 1.15 |
| V729A | 1.13 |
| V483P | 1.12 |
| S685A | 1.12 |
| N93T | 1.11 |
| G200P | 1.10 |
| C310A | 1.09 |
| D389P | 1.08 |
| V325M | 1.07 |

TABLE 3
PPDS1 Derivatives

| PPDS1 derivatives | Fold improvement |
|---|---|
| T108N | 3.98 |
| I212F | 3.30 |
| K338G | 3.18 |
| D135E | 3.16 |
| S68P | 2.85 |
| V150P | 2.75 |
| F167H | 2.62 |
| L283M | 2.55 |
| S192A | 2.40 |
| H482R | 2.27 |
| R347Q | 2.12 |
| M390L | 2.12 |
| R243K | 2.01 |
| L346I | 1.97 |
| L292I | 1.97 |
| V329M | 1.91 |
| Q278E | 1.77 |
| N58E | 1.76 |
| G152A | 1.55 |
| E202P | 1.53 |
| M153L | 1.50 |
| V248I | 1.47 |
| I95V | 1.44 |
| L96F | 1.26 |
| F317L | 1.22 |
| R85K | 1.21 |
| N333K | 1.17 |
| M144L | 1.16 |
| N277D | 1.10 |
| I362L | 1.07 |

TABLE 4
PPTS1 Derivatives

| PPTS1 derivatives | Fold improvement |
|---|---|
| G294T | 3.10 |
| S166K | 1.89 |
| C472H | 1.83 |
| K252Q | 1.76 |
| M259L | 1.73 |
| V239I | 1.68 |
| A323P | 1.61 |
| E324G | 1.60 |
| Q249E | 1.52 |
| V278I | 1.48 |
| I412V | 1.44 |
| R334K | 1.43 |
| V359A | 1.41 |
| I369T | 1.40 |
| V431I | 1.39 |
| R244K | 1.38 |
| K362D | 1.35 |
| T250P | 1.34 |
| N463K | 1.31 |
| K247L | 1.29 |
| S328N | 1.26 |
| V358E | 1.23 |
| E176K | 1.20 |
| M407A | 1.20 |
| N367G | 1.20 |
| S364T | 1.16 |
| A120S | 1.16 |
| F409Y | 1.14 |
| K391P | 1.14 |
| K146R | 1.13 |
| L187F | 1.12 |
| F147Y | 1.11 |
| A113S | 1.10 |
| L215I | 1.09 |
| 198L | 1.08 |
| F217L | 1.07 |
| W185R | 1.07 |

1. A method for producing dammarenediol or a derivative thereof, comprising:
providing a microbial host cell expressing a heterologous biosynthetic pathway producing dammarenediol or a derivative thereof, the heterologous biosynthetic pathway comprising one or more of:
a dammarenediol synthase (DDS) enzyme comprising an amino acid sequence that has at least about 70% sequence identity to the amino acid sequence of SEQ ID NO: 3, SEQ ID NO: 4, or SEQ ID NO: 9;
a protopanaxadiol synthase (PPDS) enzyme comprising an amino acid sequence that has at least 70% sequence identity to the amino acid sequence of SEQ ID NO: 10, SEQ ID NO: 11, SEQ ID NO: 12, SEQ ID NO: 13, SEQ ID NO: 14, SEQ ID NO: 15, or SEQ ID NO: 16; and
a protopanaxatriol synthase (PPTS) enzyme comprising an amino acid sequence that has at least 70% sequence identity to the amino acid sequence of SEQ ID NO: 17, SEQ ID NO: 18, SEQ ID NO: 19, SEQ ID NO: 20, and SEQ ID NO: 21.

2. The method of claim 1, wherein the DDS enzyme comprises an amino acid sequence having at least 80% sequence identity, or at least 85% sequence identity, or at least 90% sequence identity, or at least 95% sequence identity, or at least 97% sequence identity, or at least 98% sequence identity, or at least 99% sequence identity to the amino acid sequence of SEQ ID NO: 3, SEQ ID NO: 4, or SEQ ID NO: 9.

3. The method of claim 1, wherein the DDS enzyme comprises an amino acid sequence having at least 70% sequence identity, or at least 80% sequence identity, or at least 85% sequence identity, or at least 90% sequence identity, or at least 95% sequence identity, or at least 97% sequence identity, or at least 98% sequence identity, or at least 99% sequence identity to the amino acid sequence of SEQ ID NO: 3.

4. The method of claim 3, wherein the DDS enzyme comprises from 1 to 20, or from 1 to 10, or from 1 to 5 amino acid modifications independently selected from substitutions, deletions, and insertions with respect to SEQ ID NO: 3.

5. The method of claim 3 or claim 4, wherein the DDS enzyme comprises one or more substitutions at positions selected from 606, 628, and 632 with respect to SEQ ID NO: 3.

6. The method of claim 5, wherein the DDS enzyme comprises one or more substitutions selected from N606I, N606L, N606V, T628A, T628V, T628G, F632L, F632I, F632V, and F632A with respect to SEQ ID NO: 3.

7. The method of claim 6, wherein the DDS enzyme comprises one or more substitutions selected from N606I, T628A, and F632L with respect to SEQ ID NO: 3, and wherein the DDS enzyme optionally comprises the amino acid sequence of SEQ ID NO: 5.

8. The method of claim 3 or claim 4, wherein the DDS enzyme comprises one or more substitutions at positions selected from 365, 369, and 461 with respect to SEQ ID NO: 3.

9. The method of claim 8, wherein the DDS enzyme comprises one or more substitutions selected from T365E, T365D, F369Y, R461T, and R461S with respect to SEQ ID NO: 3.

10. The method of claim 9, wherein the DDS enzyme comprises one or more substitutions selected from T365E, F369Y, and R461S with respect to SEQ ID NO: 3, and wherein the DDS enzyme optionally comprises the amino acid sequence of SEQ ID NO: 6.

11. The method of claim 3 or claim 4, wherein the DDS enzyme comprises one or more substitutions at positions selected from 30, 64, and 68 with respect to SEQ ID NO: 3.

12. The method of claim 11, wherein the DDS enzyme comprises one or more substitutions selected from Q30D, Q30E, M64L, M64I, M64V, M64A, and R68M with respect to SEQ ID NO: 3.

13. The method of claim 12, wherein the DDS enzyme comprises one or more substitutions selected from Q30D, M64L, and R68M with respect to SEQ ID NO: 3, wherein the DDS enzyme optionally comprises the amino acid sequence of SEQ ID NO: 7.

14. The method of claim 3 or claim 4, wherein the DDS enzyme comprises one or more substitutions at positions selected from 425, 465, and 468 with respect to SEQ ID NO: 3.

15. The method of claim 14, wherein the DDS enzyme comprises one or more substitutions selected from L465K, L465R, L465H, C468Y, C468F, C468W, I425G, I424V, and I424A with respect to SEQ ID NO: 3.

16. The method of claim 15, wherein the DDS enzyme comprises one or more substitutions selected from L465K, C468Y, and I425A with respect to SEQ ID NO: 3, wherein the DDS enzyme optionally comprises the amino acid sequence of SEQ ID NO: 8.

17. The method of claim 3 or claim 4, wherein the DDS enzyme comprises one or more substitutions at positions corresponding to positions selected from 30, 64, 68, 365, 369, 425, 461, 465, 468, 606, 628, and 632 with respect to SEQ ID NO: 3.

18. The method of claim 17, wherein the DDS enzyme comprises at least 2, or at least 3, or at least 4 substitutions at positions corresponding to positions selected from 30, 64, 68, 365, 369, 425, 461, 465, 468, 606, 628, and 632 with respect to SEQ ID NO: 3.

19. The method of claim 3 or 4, wherein the DDS enzyme comprises an amino acid sequence that is at least 90% identical to SEQ ID NO: 7, and has one or more substitutions with respect to SEQ ID NO: 7 selected from: T364E, T364D, F368Y, R460S, R460T, L464K, L464R, C467Y, and I424A.

20. The method of claim 19, wherein the DDS enzyme comprises an amino acid sequence that is at least 90% identical to SEQ ID NO: 7, and has two, three, or more substitutions with respect to SEQ ID NO: 7 selected from: T364E, F368Y, R460S, L464K, C467Y, and I424A.

21. The method of claim 20, wherein the DDS enzyme comprises an amino acid sequence that is at least 90% identical to SEQ ID NO: 7, and has at least the following substitutions with respect to SEQ ID NO: 7: T364E, F368Y, R460S, L464K, C467Y, and I424A, and wherein the DDS enzyme optionally has the amino acid sequence of SEQ ID NO: 81.

22. The method of claim 3, wherein the DDS enzyme comprises an amino acid sequence that is at least 90% identical, or at least 95% identical, or at least 97% identical, or at least 98% identical to SEQ ID NO: 81.

23. The method of claim 22, wherein the DDS enzymes comprises one or more mutations with respect to SEQ ID NO: 81 listed in Table 1.

24. The method of claim 23, wherein the DDS enzyme comprises two, three, four, five, or more mutations with respect to SEQ ID NO: 81 listed in Table 1.

25. The method of claim 24, wherein the DDS enzyme has one or more of the following mutations with respect to SEQ ID NO: 81: Y49F, S181T, deletion of amino acids L195-E197, S198P, E238S, I407V, D507E, R637K, and M695I, and the DDS enzyme optionally comprises the amino acid sequence of SEQ ID NO: 82.

26. The method of claim 3, wherein the DDS enzyme comprises an amino acid sequence that is at least 90% identical, or at least 95% identical, or at least 97% identical, or at least 98% identical to SEQ ID NO: 82.

27. The method of claim 26, wherein the DDS enzyme comprises one or more mutations with respect to SEQ ID NO: 82 listed in Table 2.

28. The method of claim 27, wherein the DDS enzyme comprises two, three, four, five, or more mutations with respect to SEQ ID NO: 82 listed in Table 2.

29. The method of claim 26, wherein the DDS enzyme comprises one or more mutations with respect to SEQ ID NO: 82 selected from: F649L, F649V, F649I, F649A, L548F, Q149E, Q149D, A120S, A120T, G573A, G573L, S380A, S380G, and A256G.

30. The method of claim 29, wherein the DDS enzyme comprises two, three, four, five, or all mutations with respect to SEQ ID NO: 82 selected from: F649L, L548F, Q149E, A120S, G573A, S380A, and A256G.

31. The method of claim 1, wherein the heterologous biosynthetic pathway comprises a DDS enzyme comprising an amino acid sequence that is at least 90%, or at least 95%, or at least 97%, or at least 98%, or at least 99% identical to SEQ ID NO: 85.

32. The method of any one of claims 1 to 31, wherein the heterologous biosynthetic pathway comprises a PPDS enzyme comprising an amino acid sequence that has at least 70% sequence identity to the amino acid sequence of SEQ ID NO: 10, SEQ ID NO: 11, SEQ ID NO: 12, SEQ ID NO: 13, SEQ ID NO: 14, SEQ ID NO: 14, SEQ ID NO: 15, or SEQ ID NO: 16.

33. The method of claim 32, wherein the PPDS enzyme comprises an amino acid sequence having at least 70%, or at least 80%, or at least 85%, or at least 90%, or at least 95%, or at least 98%, or at least 99% sequence identity to an amino acid sequence selected from SEQ ID NOs: 10, 11, 12 and 16.

34. The method of claim 33, wherein the PPDS enzyme comprises an amino acid sequence having at least 70%, or at least 80%, or at least 85%, or at least 90%, or at least 95%, or at least 98%, or at least 99% sequence identity to the amino acid sequence of SEQ ID NO: 10.

35. The method of claim 34, wherein the PPDS enzyme comprises from 1 to 20, or from 1 to 10, or from 1 to 5 amino acid substitutions with respect to SEQ ID NO: 10.

36. The method of claim 34 or claim 35, wherein the PPDS enzyme comprises one or more substitutions selected from N58D, N58E, S68P, R85K, I95V, L96F, L96W, L96Y, T108N, T108Q, D135E, M144L, M144V, M144I, V150P, G152A, G152L, G152I, G152V, M153L, M153I, M153V, M153A, F167H, S192A, S192G, E202P, I212F, R243K, R243H, V248I, V248L,N277D, N277E, Q278E, Q278D, L283M, L292I, L292V, L292A, F317L, F317I, F317V, F317A, V329M, N333K, N333R, K338G, K338A, L346I, R347Q, R347N, I362L, I362V, I362A, M390L, M390I, M390V, M390A, H482R, and H482K with respect to SEQ ID NO: 10.

37. The method of claim 36, wherein the PPDS enzyme comprises at least 2, or at least 3, or at least 4, or at least 5, or at least 8, or at least 10 amino acid substitutions at positions disclosed in Table 3.

38. The method of claim 37, wherein the PPDS enzyme comprises at least one, two, three, four, five, six, seven, eight, or all of the following mutations with respect to SEQ ID NO: 10: T108N, I212F, K338G, D135E, S68P, V150P, F167H, L283M, H482R, R347Q, M390L, R243K, L292I, V329M, Q278E, and N58E.

39. The method of claim 38, wherein the PPDS comprises the amino acid sequence of SEQ ID NO: 83, optionally with from 1 to 10 or from 1 to 5 amino acid modifications independently selected from substitutions, insertions, and deletions.

40. The method of claim 1, wherein the heterologous biosynthetic pathway comprises a PPDS enzyme comprising an amino acid sequence that is at least 90%, or at least 95%, or at least 97%, or at least 98%, or at least 99% identical to SEQ ID NO: 83.

41. The method of any one of claims 1 to 40, wherein the heterologous biosynthetic pathway comprises a PPTS enzyme comprising an amino acid sequence that has at least 70% sequence identity to an amino acid sequence selected from SEQ ID NO: 17, SEQ ID NO: 18, SEQ ID NO: 19, SEQ ID NO: 20 and SEQ ID NO: 21.

42. The method of claim 41, wherein the PPTS enzyme comprises an amino acid sequence that has at least 70% sequence identity, or at least 80% sequence identity, or at least 85% sequence identity, or at least 90% sequence identity, or at least 95% sequence identity, or at least 97% sequence identity, or at least 98% sequence identity to an amino acid sequence selected from SEQ ID NO: 17, SEQ ID NO: 18, SEQ ID NO: 19, SEQ ID NO: 20 and SEQ ID NO: 21.

43. The method of claim 42, wherein the PPTS enzyme comprises an amino acid sequence having at least 70%, or at least 80%, or at least 85%, or at least 90%, or at least 95%, or at least 98%, or at least 99% sequence identity to the amino acid sequence of SEQ ID NO: 17.

44. The method of claim 43, wherein the PPTS enzyme comprises from 1 to 20, or from 1 to 10, or from 1 to 5 amino acid substitutions with respect to SEQ ID NO: 17.

45. The method of claim 43 or 44, wherein the PPTS enzyme comprises one or more substitutions with respect to SEQ ID NO: 17 selected from: 198L, 198V, 198A, A113S, A120S, A120T, K146R, F147Y, S166K, S166R, E176K, E176R, W185R, W185K, L187F, L187Y, L187W, L215I, F217L, F217I, F217V, F217A, V239I, V239L, V239A, R244K, K247L, K247V, K247I, K247A, Q249E, Q249D, T250P, K252Q, K252N, M259L, M259I, M259V, V278I, G294T, G294S, A323P, E324G, E324A, S328N, S328Q, R334K, V358E, V358D, V359A, V359G, K362D, K362E, S364T, N367G, N367A, I369T, I369S, K391P, M407A, M407G, F409Y, I412V, F426Y, V431I, V431L, N463K, N463R, and C472H.

46. The method of claim 45, wherein the PPTS enzyme comprises at least 2, or at least 3, or at least 4, or at least 5, or at least 8, or at least 10 amino acid substitutions with respect to SEQ ID NO: 17 as listed in Table 4.

47. The method of claim 46, wherein the PPTS enzyme comprises at least two, at least 3, at least 4, at least 5, or all amino acid substitutions selected from SEQ ID NO: 17 selected from: G294T, S166K, C472H, K252Q, V239I, A323P, I412V, I369T, K362D, and T250P.

48. The method of claim 47, wherein the PPTS comprises the amino acid sequence of SEQ ID NO: 84, optionally with from 1 to 10 or from 1 to 5 amino acid modifications independently selected from substitutions, insertions, and deletions.

49. The method of claim 1, wherein the heterologous biosynthetic pathway comprises a PPTS enzyme comprising an amino acid sequence that is at least 90%, or at least 95%, or at least 97%, or at least 98%, or at least 99% identical to SEQ ID NO: 84.

50. The method of any one of claims 1 to 49, wherein the heterologous biosynthetic pathway further comprises one or more uridine diphosphate-dependent glycosyltransferase (UGT) enzymes that glycosylate one or more of dammaranediol, protopanaxadiol and protopanaxatriol.

51. The method of claim 50, wherein the UGT enzyme(s) are capable of catalyzing glycosylation of C₃—OH, C₆—OH, and/or C₂₀—OH, and optionally one or more branching glycosylations.

52. The method of any one of claims 1 to 51, wherein the heterologous biosynthetic pathway further comprises a squalene synthase (SQS) enzyme.

53. The method of claim 52, wherein the SQS comprises an amino acid sequence having at least 70%, or at least 80%, or at least 85%, or at least 90%, or at least 95%, or at least 98%, or at least 99% sequence identity to an amino acid sequence selected from SEQ ID NOs: 1 and 23-38.

54. The method of claim 53, wherein the SQS comprises an amino acid sequence having at least 70%, or at least 80%, or at least 85%, or at least 90%, or at least 95%, or at least 98%, or at least 99% sequence identity to an amino acid sequence selected from SEQ ID NO: 1.

55. The method of any one of claims 1 to 54, wherein the heterologous biosynthetic pathway further comprises a squalene epoxidase (SQE).

56. The method of claim 55, wherein the SQE comprises an amino acid sequence having at least 70%, or at least 80%, or at least 85%, or at least 90%, or at least 95%, or at least 98%, or at least 99% sequence identity to an amino acid sequence selected from SEQ ID NOs: 2 and 39-70.

57. The method of claim 56, wherein the SQE enzyme comprises an amino acid sequence having at least 70%, or at least 80%, or at least 85%, or at least 90%, or at least 95%, or at least 98%, or at least 99% sequence identity to the amino acid sequence of SEQ ID NO: 2.

58. The method of any one of claims 1 to 57, wherein the microbial host cell expresses an enzymatic pathway that produces iso-pentenyl pyrophosphate (IPP) and dimethylallyl pyrophosphate (DMAPP).

59. The method of claim 58, wherein the enzymatic pathway is a methylerythritol phosphate (MEP) pathway and/or a mevalonic acid (MVA) pathway.

60. The method of claim 59, wherein the microbial host cell is a bacterium that produces increased MEP pathway products.

61. The method of claim 59 or 60, wherein the bacterium is selected from Escherichia coli, Bacillus subtilis, Corynebacterium glutamicum, Rhodobacter capsulatus, Rhodobacter sphaeroides, Zymomonas mobilis, Vibrio natriegens, and Pseudomonas putida.

62. The method of claim 61, wherein the microbial host cell is E. coli.

63. The method of any one of claims 1 to 62, wherein the microbial host is a yeast, optionally selected from a species of Saccharomyces, Pichia, or Yarrowia, and which is optionally Saccharomyces cerevisiae, Pichia pastoris, and Yarrowia lipolytica.

64. The method of any one of claims 1 to 63, wherein the microbial host cell is cultured in a carbon source comprising glucose, sucrose, fructose, xylose, and/or glycerol.

65. The method of claim 64, wherein culture conditions are selected from aerobic, microaerobic, and anaerobic.

66. The method of claim 65, wherein the microbial host cell is cultured at a temperature in the range of about 22° C. to about 37° C., or about 27° C. to about 37° C., or about 30° C. to about 37° C.

67. The method of any one of claims 1 to 66, wherein dammaranediol, protopanaxadiol, protopanaxatriol or a glycosylated derivative thereof is recovered from the culture.

68. A microbial host cell producing dammarenediol or a derivative thereof, the microbial host cell expressing a heterologous biosynthetic pathway producing dammarenediol or a derivative thereof, the heterologous biosynthetic pathway comprising one or more of:
a dammarenediol synthase (DDS) enzyme comprising an amino acid sequence that has at least about 70% sequence identity to the amino acid sequence of SEQ ID NO: 3, SEQ ID NO: 4, or SEQ ID NO: 9;
a protopanaxadiol synthase (PPDS) enzyme comprising an amino acid sequence that has at least 70% sequence identity to the amino acid sequence of SEQ ID NO: 10, SEQ ID NO: 11, SEQ ID NO: 12, SEQ ID NO: 13, SEQ ID NO: 14, SEQ ID NO: 14, SEQ ID NO: 15, or SEQ ID NO: 16; and
a protopanaxatriol synthase (PPTS) enzyme comprising an amino acid sequence that has at least 70% sequence identity to the amino acid sequence of SEQ ID NO: 17, SEQ ID NO: 18, SEQ ID NO: 19, SEQ ID NO: 20, and SEQ ID NO: 21.

69. The microbial host cell of claim 68, wherein the DDS enzyme comprises an amino acid sequence having at least 80% sequence identity, or at least 85% sequence identity, or at least 90% sequence identity, or at least 95% sequence identity, or at least 97% sequence identity, or at least 98% sequence identity, or at least 99% sequence identity to the amino acid sequence of SEQ ID NO: 3, SEQ ID NO: 4, or SEQ ID NO: 9.

70. The microbial host cell of claim 68, wherein the DDS enzyme comprises an amino acid sequence having at least 70% sequence identity, or at least 80% sequence identity, or at least 85% sequence identity, or at least 90% sequence identity, or at least 95% sequence identity, or at least 97% sequence identity, or at least 98% sequence identity, or at least 99% sequence identity to SEQ ID NO: 3.

71. The microbial host cell of claim 70, wherein the DDS enzyme comprises from 1 to 20, or from 1 to 10, or from 1 to 5 amino acid substitutions with respect to SEQ ID NO: 3.

72. The microbial host cell of claim 70 or claim 71, wherein the DDS enzyme comprises one or more substitutions at positions selected from 30, 64, 68, 365, 369, 425, 461, 465, 468, 606, 628, and 632 with respect to SEQ ID NO: 3.

73. The microbial host cell of claim 72, wherein the DDS enzyme comprises at least 2, or at least 3, or at least 4, or more substitutions at positions selected from 30, 64, 68, 365, 369, 425, 461, 465, 468, 606, 628, and 632 with respect to SEQ ID NO: 3.

74. The microbial host cell of claim 73, wherein the DDS enzyme comprises one or more substitutions selected from N606I, N606L, N606V, T628A, T628V, T628G, F632L, F632I, F632V, and F632A with respect to SEQ ID NO: 3.

75. The microbial host cell of claim 74, wherein the DDS enzyme comprises one or more substitutions selected from N606I, T628A, and F632L with respect to SEQ ID NO: 3, and wherein the DDS enzyme optionally comprises the amino acid sequence of SEQ ID NO: 5.

76. The microbial host cell of any one of claims 71 to 75, wherein the DDS enzyme comprises one or more substitutions at positions selected from 365, 369, and 461 with respect to SEQ ID NO: 3.

77. The microbial host cell of claim 76, wherein the DDS enzyme comprises one or more substitutions selected from T365E, T365D, F369Y, R461T, and R461S with respect to SEQ ID NO: 3.

78. The microbial host cell of claim 77, wherein the DDS enzyme comprises one or more substitutions selected from T365E, F369Y, and R461S with respect to SEQ ID NO: 3, and wherein the DDS enzyme optionally comprises the amino acid sequence of SEQ ID NO: 6.

79. The microbial host cell of any one of claims 71 to 78, wherein the DDS enzyme comprises one or more substitutions at positions selected from 30, 64, and 68 with respect to SEQ ID NO: 3.

80. The microbial host cell of claim 79, wherein the DDS enzyme comprises one or more substitutions selected from Q30D, Q30E, M64L, M64I, M64V, M64A, and R68M with respect to SEQ ID NO: 3.

81. The microbial host cell of claim 80, wherein the DDS enzyme comprises one or more substitutions selected from Q30D, M64L, and R68M with respect to SEQ ID NO: 3, wherein the DDS enzyme optionally comprises the amino acid sequence of SEQ ID NO: 7.

82. The microbial host cell of any one of claims 71 to 81, wherein the DDS enzyme comprises one or more substitutions at positions selected from 425, 465, and 468 with respect to SEQ ID NO: 3.

83. The microbial host cell of claim 82, wherein the DDS enzyme comprises one or more substitutions selected from L465K, L465R, L465H, C468Y, C468F, C468W, I425G, I425V, and I425A with respect to SEQ ID NO: 3.

84. The microbial host cell of claim 83, wherein the DDS enzyme comprises one or more substitutions selected from L465K, C468Y, and I425A with respect to SEQ ID NO: 3, wherein the DDS enzyme optionally comprises the amino acid sequence of SEQ ID NO: 8.

85. The microbial host cell of claim 70 or 71, wherein the DDS enzyme comprises an amino acid sequence that is at least 90% identical to SEQ ID NO: 7, and has one or more substitutions with respect to SEQ ID NO: 7 selected from: T364E, T364D, F368Y, R460S, R460T, L464K, L464R, C467Y, and I424A.

86. The microbial host cell of claim 85, wherein the DDS enzyme comprises an amino acid sequence that is at least 90% identical to SEQ ID NO: 7, and has two, three, or more substitutions with respect to SEQ ID NO: 7 selected from: T364E, F368Y, R460S, L464K, C467Y, and I424A.

87. The microbial host cell of claim 86, wherein the DDS enzyme comprises an amino acid sequence that is at least 90% identical to SEQ ID NO: 7, and has at least the following substitutions with respect to SEQ ID NO: 7: T364E, F368Y, R460S, L464K, C467Y, and I424A, and wherein the DDS enzyme optionally has the amino acid sequence of SEQ ID NO: 81.

88. The microbial host cell of claim 68, wherein the DDS enzyme comprises an amino acid sequence that is at least 90% identical, or at least 95% identical, or at least 97% identical, or at least 98% identical to SEQ ID NO: 81.

89. The microbial host cell of claim 88, wherein the DDS enzymes comprises one or more mutations with respect to SEQ ID NO: 81 listed in Table 1.

90. The microbial host cell of claim 87, wherein the DDS enzymes comprises two, three, four, five, or more mutations with respect to SEQ ID NO: 81 listed in Table 1.

91. The microbial host cell of claim 89, wherein the DDS enzyme has one or more of the following mutations with respect to SEQ ID NO: 81: Y49F, S181T, deletion of amino acids L195-E197, S198P, E238S, I407V, D507E, R637K, and M695I, and the DDS enzyme optionally comprises the amino acid sequence of SEQ ID NO: 82.

92. The microbial host cell of claim 68, wherein the heterologous pathway comprises DDS enzyme comprising an amino acid sequence that is at least 90% identical, or at least 95% identical, or at least 97% identical, or at least 98% identical to SEQ ID NO: 82.

93. The microbial host cell of claim 92, wherein the DDS enzymes comprises one or more mutations with respect to SEQ ID NO: 82 listed in Table 2.

94. The microbial host cell of claim 93, wherein the DDS enzymes comprises two, three, four, five, or more mutations with respect to SEQ ID NO: 82 listed in Table 2.

95. The microbial host cell of claim 94, wherein the DDS enzyme comprises one or more mutations with respect to SEQ ID NO: 82 selected from: F649L, F649V, F649I, F649A, L548F, Q149E, Q149D, A120S, A120T, G573A, G573L, S380A, S380G, and A256G.

96. The microbial host cell of claim 95, wherein the DDS enzyme comprises two, three, four, five, or all mutations with respect to SEQ ID NO: 82 selected from: F649L, L548F, Q149E, A120S, G573A, S380A, and A256G.

97. The microbial host cell of claim 68, wherein the heterologous biosynthetic pathway comprises a DDS enzyme comprising an amino acid sequence that is at least 90%, or at least 95%, or at least 97%, or at least 98%, or at least 99% identical to SEQ ID NO: 85.

98. The microbial host cell of any one of claims 68 to 97, wherein the heterologous biosynthetic pathway comprises a PPDS enzyme comprising an amino acid sequence that has at least 70% sequence identity to the amino acid sequence of SEQ ID NO: 10, SEQ ID NO: 11, SEQ ID NO: 12, SEQ ID NO: 13, SEQ ID NO: 14, SEQ ID NO: 14, SEQ ID NO: 15, or SEQ ID NO: 16.

99. The microbial host cell of claim 98, wherein the PPDS enzyme comprises an amino acid sequence having at least 70%, or at least 80%, or at least 85%, or at least 90%, or at least 95%, or at least 98%, or at least 99% sequence identity to an amino acid sequence selected from SEQ ID NOs: 10, 11, 12 and 16.

100. The microbial host cell of claim 98, wherein the PPDS enzyme comprises an amino acid sequence having at least 70%, or at least 80%, or at least 85%, or at least 90%, or at least 95%, or at least 98%, or at least 99% sequence identity to the amino acid sequence of SEQ ID NO: 10.

101. The microbial host cell of claim 100, wherein the PPDS enzyme comprises from 1 to 20, or from 1 to 10, or from 1 to 5 amino acid substitutions with respect to SEQ ID NO: 10.

102. The microbial host cell of claim 100 or claim 101, wherein the PPDS enzyme comprises one or more substitutions selected from N58D, N58E, S68P, R85K, I95V, L96F, L96W, L96Y, T108N, T108Q, D135E, M144L, M144V, M144I, V150P, G152A, G152L, G152I, G152V, M153L, M153I, M153V, M153A, F167H, S192A, S192G, E202P, I212F, R243K, R243H, V248I, V248L,N277D, N277E, Q278E, Q278D, L283M, L292I, L292V, L292A, F317L, F317I, F317V, F317A, V329M, N333K, N333R, K338G, K338A, L346I, R347Q, R347N, I362L, I362V, I362A, M390L, M390I, M390V, M390A, H482R, and H482K with respect to SEQ ID NO: 10.

103. The microbial host cell of claim 102, wherein the PPDS enzyme comprises at least 2, or at least 3, or at least 4, or at least 5, or at least 8, or at least 10 amino acid substitutions listed in Table 3.

104. The microbial host cell of claim 103, wherein the PPDS enzyme comprises at least one, two, three, four, five, six, seven, eight, or all of the following mutations with respect to SEQ ID NO: 10: T108N, I212F, K338G, D135E, S68P, V150P, F167H, L283M, H482R, R347Q, M390L, R243K, L292I, V329M, Q278E, and N58E.

105. The microbial host cell of claim 104, wherein the PPDS comprises the amino acid sequence of SEQ ID NO: 83, optionally with from 1 to 10 or from 1 to 5 amino acid modifications independently selected from substitutions, insertions, and deletions.

106. The microbial host cell of claim 68, wherein the heterologous biosynthetic pathway comprises a PPDS enzyme comprising an amino acid sequence that is at least 90%, or at least 95%, or at least 97%, or at least 98%, or at least 99% identical to SEQ ID NO: 83.

107. The microbial host cell of any one of claims 68 to 106, wherein the heterologous biosynthetic pathway comprises a PPTS enzyme comprising an amino acid sequence that has at least 70% sequence identity to an amino acid sequence selected from SEQ ID NO: 17, SEQ ID NO: 18, SEQ ID NO: 19, SEQ ID NO: 20 and SEQ ID NO: 21.

108. The microbial host cell of claim 106, wherein the PPTS enzyme comprises an amino acid sequence that has at least 70% sequence identity, or at least 80% sequence identity, or at least 85% sequence identity, or at least 90% sequence identity, or at least 95% sequence identity, or at least 97% sequence identity, or at least 98% sequence identity to an amino acid sequence selected from SEQ ID NO: 17, SEQ ID NO: 18, SEQ ID NO: 19, SEQ ID NO: 20 and SEQ ID NO: 21.

109. The microbial host cell of claim 107, wherein the PPTS enzyme comprises an amino acid sequence having at least 70%, or at least 80%, or at least 85%, or at least 90%, or at least 95%, or at least 98%, or at least 99% sequence identity to the amino acid sequence of SEQ ID NO: 17.

110. The microbial host cell of claim 109, wherein the PPTS enzyme comprises from 1 to 20, or from 1 to 10, or from 1 to 5 amino acid substitutions with respect to SEQ ID NO: 17.

111. The microbial host cell of claim 109 or 110, wherein the PPTS enzyme comprises one or more substitutions with respect to SEQ ID NO: 17 selected from: I98L, I98V, I98A, A113S, A120S, A120T, K146R, F147Y, S166K, S166R, E176K, E176R, W185R, W185K, L187F, L187Y, L187W, L215I, F217L, F217I, F217V, F217A, V239I, V239L, V239A, R244K, K247L, K247V, K247I, K247A, Q249E, Q249D, T250P, K252Q, K252N, M259L, M259I, M259V, V278I, G294T, G294S, A323P, E324G, E324A, S328N, S328Q, R334K, V358E, V358D, V359A, V359G, K362D, K362E, S364T, N367G, N367A, I369T, I369S, K391P, M407A, M407G, F409Y, I412V, F426Y, V431I, V431L, N463K, N463R, and C472H.

112. The microbial host cell of claim 111, wherein the PPTS enzyme comprises at least 2, or at least 3, or at least 4, or at least 5, or at least 8, or at least 10 amino acid substitutions with respect to SEQ ID NO: 17 as listed in Table 4.

113. The microbial host cell of claim 112, wherein the PPTS enzyme comprises at least two, at least 3, at least 4, at least 5, or all amino acid substitutions selected from SEQ ID NO: 17 selected from: G294T, S166K, C472H, K252Q, V239I, A323P, I412V, I369T, K362D, and T250P.

114. The microbial of claim 113, wherein the PPTS comprises the amino acid sequence of SEQ ID NO: 84, optionally with from 1 to 10 or from 1 to 5 amino acid modifications independently selected from substitutions, insertions, and deletions.

115. The microbial host cell of claim 68, wherein the heterologous biosynthetic pathway comprises a PPTS enzyme comprising an amino acid sequence that is at least 90%, or at least 95%, or at least 97%, or at least 98%, or at least 99% identical to SEQ ID NO: 84.

116. The microbial host cell of any one of claims 68 to 115, wherein the heterologous biosynthetic pathway further comprises one or more uridine diphosphate-dependent glycosyltransferase (UGT) enzymes that glycosylate one or more of dammaranediol, protopanaxadiol and protopanaxatriol.

117. The microbial host cell of claim 116, wherein the UGT enzyme(s) are capable of catalyzing glycosylation of C₃—OH, C₆—OH, and/or C₂₀—OH, and optionally one or more branching glycosylations.

118. The microbial host cell of any one of claims 68 to 117, wherein the heterologous biosynthetic pathway further comprises a squalene synthase (SQS) enzyme.

119. The microbial host cell of claim 118, wherein the SQS comprises an amino acid sequence having at least 70%, or at least 80%, or at least 85%, or at least 90%, or at least 95%, or at least 98%, or at least 99% sequence identity to an amino acid sequence selected from SEQ ID NOs: 1 and 23-38.

120. The microbial host cell of claim 119, wherein the SQS comprises an amino acid sequence having at least 70%, or at least 80%, or at least 85%, or at least 90%, or at least 95%, or at least 98%, or at least 99% sequence identity to an amino acid sequence selected from SEQ ID NO: 1.

121. The microbial host cell of any one of claims 68 to 120, wherein the heterologous biosynthetic pathway further comprises a squalene epoxidase (SQE).

122. The microbial host cell of claim 121, wherein the SQE comprises an amino acid sequence having at least 70%, or at least 80%, or at least 85%, or at least 90%, or at least 95%, or at least 98%, or at least 99% sequence identity to an amino acid sequence selected from SEQ ID NOs: 2 and 39-70.

123. The microbial host cell of claim 121, wherein the SQE enzyme comprises an amino acid sequence having at least 70%, or at least 80%, or at least 85%, or at least 90%, or at least 95%, or at least 98%, or at least 99% sequence identity to the amino acid sequence of SEQ ID NO: 2.

124. The microbial host cell of any one of claims 68 to 123, wherein the microbial host cell expresses an enzymatic pathway that produces iso-pentenyl pyrophosphate (IPP) and dimethylallyl pyrophosphate (DMAPP).

125. The microbial host cell of claim 124, wherein the enzymatic pathway is a methylerythritol phosphate (MEP) pathway and/or a mevalonic acid (MVA) pathway.

126. The microbial host cell of claim 125, wherein the microbial host cell is a bacterium that produces increased MEP pathway products.

127. The microbial host cell of claim 125 or 126, wherein the bacterium is selected from Escherichia coli, Bacillus subtilis, Corynebacterium glutamicum, Rhodobacter capsulatus, Rhodobacter sphaeroides, Zymomonas mobilis, Vibrio natriegens, and Pseudomonas putida.

128. The microbial host cell of claim 127, wherein the microbial host cell is E. coli.

129. The microbial host cell of any one of claims 68 to 125, wherein the microbial host is a yeast, optionally selected from a species of Saccharomyces, Pichia, or Yarrowia, and which is optionally Saccharomyces cerevisiae, Pichia pastoris, and Yarrowia lipolytica.

130. A dammarenediol synthase (DDS) enzyme comprising an amino acid sequence that is at least 90% or at least 95%, or at least 97%, or at least 98% identical to SEQ ID NO: 3, wherein the DDS has one or more of:
one or more substitutions selected from N606I, N606L, N606V, T628A, T628V, T628G, F632L, F632I, F632V, and F632A with respect to SEQ ID NO: 3;
one or more substitutions selected from T365E, T365D, F369Y, R461T, and R461S with respect to SEQ ID NO: 3;
one or more substitutions selected from Q30D, Q30E, M64L, M64I, M64V, M64A, and R68M with respect to SEQ ID NO: 3; and
one or more substitutions selected from L465K, L465R, L465H, C468Y, C468F, C468W, I425G, I425V, and I425A with respect to SEQ ID NO: 3.

131. The DDS enzyme of claim 130, wherein the DDS enzyme comprises an amino acid sequence that is at least 90% identical to SEQ ID NO: 7, and optionally has at least the following substitutions with respect to SEQ ID NO: 7: T364E, F368Y, R460S, L464K, C467Y, and I424A, and wherein the DDS enzyme optionally has the amino acid sequence of SEQ ID NO: 81.

132. The DDS enzyme of claim 131, wherein the DDS enzymes comprises one or more mutations with respect to SEQ ID NO: 81 listed in Table 1, optionally wherein the DDS enzymes comprises two, three, four, five, or more mutations with respect to SEQ ID NO: 81 listed in Table 1.

133. The DDS enzyme of claim 132, wherein the DDS enzyme has one or more of the following mutations with respect to SEQ ID NO: 81: Y49F, S181T, deletion of amino acids L195-E197, S198P, E238S, I407V, D507E, R637K, and M695I, and the DDS enzyme optionally comprises the amino acid sequence of SEQ ID NO: 82.

134. The DDS enzyme of claim 133, wherein the DDS enzymes comprises one or more mutations with respect to SEQ ID NO: 82 listed in Table 2, optionally wherein the DDS enzymes comprises two, three, four, five, or more mutations with respect to SEQ ID NO: 82 listed in Table 2.

135. The DDS enzyme of claim 134, wherein the DDS enzyme comprises one or more mutations with respect to SEQ ID NO: 82 selected from: F649L, F649V, F649I, F649A, L548F, Q149E, Q149D, A120S, A120T, G573A, G573L, S380A, S380G, and A256G, optionally wherein the DDS enzyme comprises two, three, four, five, or all mutations with respect to SEQ ID NO: 82 selected from: F649L, L548F, Q149E, A120S, G573A, S380A, and A256G.

136. The DDS enzyme of claim 130, wherein the DDS enzyme comprises an amino acid sequence that is at least 95%, or at least 97%, or at least 98%, or at least 99% identical to SEQ ID NO: 85.

137. A protopanaxadiol synthase (PPDS) enzyme comprising an amino acid sequence having at least 90%, or at least 95%, or at least 98%, or at least 99% sequence identity to the amino acid sequence of SEQ ID NO: 10, wherein the PPDS enzyme comprises one or more substitutions selected from N58D, N58E, S68P, R85K, I95V, L96F, L96W, L96Y, T108N, T108Q, D135E, M144L, M144V, M144I, V150P, G152A, G152L, G152I, G152V, M153L, M153I, M153V, M153A, F167H, S192A, S192G, E202P, I212F, R243K, R243H, V248I, V248L,N277D, N277E, Q278E, Q278D, L283M, L292I, L292V, L292A, F317L, F317I, F317V, F317A, V329M, N333K, N333R, K338G, K338A, L346I, R347Q, R347N, I362L, I362V, I362A, M390L, M390I, M390V, M390A, H482R, and H482K with respect to SEQ ID NO: 10.

138. The PPDS enzyme of claim 137, wherein the PPDS enzyme comprises at least 2, or at least 3, or at least 4, or at least 5, or at least 8, or at least 10 amino acid substitutions with respect to SEQ ID NO: 10 listed in Table 3.

139. The PPDS enzyme of claim 138, wherein the PPDS enzyme comprises at least one, two, three, four, five, six, seven, eight, or all of the following mutations with respect to SEQ ID NO: 10: T108N, I212F, K338G, D135E, S68P, V150P, F167H, L283M, H482R, R347Q, M390L, R243K, L292I, V329M, Q278E, and N58E.

140. The PPDS enzyme of claim 138, wherein the PPDS comprises the amino acid sequence of SEQ ID NO: 83, optionally with from 1 to 10 or from 1 to 5 amino acid modifications independently selected from substitutions, insertions, and deletions.

141. The PPDS enzyme of claim 137, wherein the PPDS enzyme comprises an amino acid sequence that is at least 95%, or at least 97%, or at least 98%, or at least 99% identical to SEQ ID NO: 83.

142. A protopanaxatriol synthase (PPTS) enzyme, comprising an amino acid sequence having at least 90%, or at least 95%, or at least 98%, or at least 99% sequence identity to the amino acid sequence of SEQ ID NO: 17, wherein the PPTS enzyme comprises one or more substitutions with respect to SEQ ID NO: 17 selected from: 198L, 198V, 198A, A113S, A120S, A120T, K146R, F147Y, S166K, S166R, E176K, E176R, W185R, W185K, L187F, L187Y, L187W, L215I, F217L, F217I, F217V, F217A, V239I, V239L, V239A, R244K, K247L, K247V, K247I, K247A, Q249E, Q249D, T250P, K252Q, K252N, M259L, M259I, M259V, V278I, G294T, G294S, A323P, E324G, E324A, S328N, S328Q, R334K, V358E, V358D, V359A, V359G, K362D, K362E, S364T, N367G, N367A, I369T, I369S, K391P, M407A, M407G, F409Y, I412V, F426Y, V431I, V431L, N463K, N463R, and C472H, with respect to SEQ ID NO: 17.

143. The PPTS enzyme of claim 142, wherein the PPTS enzyme comprises at least 2, or at least 3, or at least 4, or at least 5, or at least 8, or at least 10 amino acid substitutions with respect to SEQ ID NO: 17 as listed in Table 4.

144. The PPTS enzyme of claim 143, wherein the PPTS enzyme comprises at least two, at least 3, at least 4, at least 5, or all amino acid substitutions selected from SEQ ID NO: 17 selected from: G294T, S166K, C472H, K252Q, V239I, A323P, I412V, I369T, K362D, and T250P.

145. The PPTS enzyme of claim 143 or 144, wherein the PPTS comprises the amino acid sequence of SEQ ID NO: 84, optionally with from 1 to 10 or from 1 to 5 amino acid modifications independently selected from substitutions, insertions, and deletions.

146. The PPTS enzyme of claim 142, wherein the PPTS enzyme comprising an amino acid sequence that is at least 95%, or at least 97%, or at least 98%, or at least 99% identical to SEQ ID NO: 84.
